(19) United States
(12) Patent Application Publication
(10) Pub. No.: US 2009/0123389 A1
(43) Pub. Date: May 14, 2009

(54) Methods for modulating Th17 cell development in the treatment and prevention of cellulite

(76) Inventors: Malcolm Whitman, Jamaica Plain, MA (US); Tracy Keller, Jamaica Plain, MA (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/228,799

(22) Filed: Aug. 15, 2008

Related U.S. Application Data
(60) Provisional application No. 60/964,957, filed on Aug. 15, 2007.

(51) Int. Cl. A61K 31/517; A61K 9/12

(52) U.S. Cl. 424/45; 514/266.22

(57) ABSTRACT

This invention relates generally to methods and compositions for modulating the development of Th17 cells for use, for example, in the treatment of cellulite.

### RELATED APPLICATIONS

This patent application claims the benefit of U.S. Provisional Patent Application Ser. No. 60/964,957, filed Aug. 15, 2007, the contents of which are herein incorporated by reference in their entirety.

### STATEMENT AS TO FEDERALLY SPONSORED RESEARCH

This invention made with U.S. Government support under Grant Number R01 HD 29468 and Grant Number R01 AI48213 from the National Institutes of Health. The Government has certain rights in the invention.

### FIELD OF THE INVENTION

This invention relates generally to methods and compositions for modulating the development of Th17 cells for use, for example, in the treatment of cellulite.

### BACKGROUND OF THE INVENTION

Cellulite is a skin condition that is characterized by dimpling and puckering of the skin of the thighs and buttocks. Cellulite is rare in men and quite common in women. Thus, cellulite poses a great cosmetic concern to a large number of people due to its prevalence.

Purported treatments for cellulite are currently being marketed, but these current treatments are often very expensive. Treatments include, e.g., include topical ointments such as aminophylline cream, which attempt to alter adipose tissue metabolism. Other treatment methods include physical and mechanical treatment methods, surgery, and laser treatment of an area exhibiting cellulite. However, no effective strategy has been described to date for the prevention or long-term management of cellulite.

Accordingly, significant need exists for methods and compositions for modulating the formation and/or presence of cellulite and other skin disorders in a subject.

### SUMMARY OF THE INVENTION

The present invention provides methods and compositions for modulating, e.g., reducing, inhibiting, treating or preventing, the formation and/or progression of cellulite and other skin disorders in a subject. The term “skin disorders” refers to a disorder that manifests itself as an aberrant or irregular appearance of the skin of an individual, but the disorder itself poses little or no health risk in and of itself and doesn't compromise or otherwise impede the body's ability to function normally and repair itself. A skin disorder to be treated or otherwise modulated by the methods and compositions described herein include disorders that affect the appearance and/or structure of any layer of the skin, e.g., the epidermis, the dermis, the hypodermis, any of the sublayers of epidermis such as the stratum basale, the stratum spinosum, the stratum granulosum, the stratum licidum, and the stratum corneum, and any combination thereof. Skin disorders to be treated or otherwise modulated by the methods and compositions provided herein include pathological and nonpathological disorders. Examples of skin disorders that are modulated, e.g., reduced, inhibited, treated or prevented, by the methods and compositions provided herein include cellulite and stretch marks (striae distensae).

The methods and compositions provided herein reduce, inhibit, treat or prevent the progression and/or formation of cellulite by specifically modulating, e.g., reducing, inhibiting, or otherwise preventing, the development and/or expansion of Th17 cells in a subject. The methods and compositions of the invention include a selective Th17 inhibitor. Selective Th17 inhibitors of the invention modulate the development and/or expansion of Th17 cells by specifically inhibiting, partially or completely, the development of naïve T cells into Th17 cells, such that the naïve cells are turned away from the Th17 lineage, which is associated with cell-mediated damage, persistent inflammation and auto-immunity. In some embodiments, the selective Th17 inhibitor alters the development of the naïve T cells away from the Th17 lineage and promotes or otherwise induces the developing T cells toward the regulatory T cells (Treg) lineage, which is thought to be anti-inflammatory and tissue protective. The selective Th17 inhibitors of the invention modulate the development and/or expansion of Th17 cells by specifically inhibiting, reducing or otherwise impeding the ability of TGFbeta to promote the expansion of Th17 cells in IL-6. Accordingly, a composition comprising a selective inhibitor of Th17 cells such as the compound of Formula I (shown below) and a second compound such as a statin, retinoic acid or an inhibitor of IL-6 or IL-21 is also within the invention. Such a combination synergistically prevents or reduces the appearance or development of cellulite.

The invention features a method for decreasing the appearance of cellulite in a subject exhibiting cellulite, by identifying a tissue treatment site in the patient, wherein the tissue treatment site includes at least one area in which the appearance of cellulite occurs, contacting the tissue treatment site with a compound that modulates the formation or progression of cellulite, where the treated tissue site exhibits a decreased contractile force exerted by vertical collagen fibers tethered between fascia and dermo-hypodermal interface within the tissue treatment site, as compared to a contractile force exerted by vertical collagen fibers in a second tissue site exhibiting cellulite in the subject. In various aspects, the method also includes exposing the tissue treatment site to a laser, and/or exposing the tissue treatment site to mechanical manipulation.

The invention provides methods for treating or preventing cellulite in a subject in need thereof by administering to the subject a compound that binds a target of halofuginone in an amount effective to modulate binding of halofuginone and the target.

The compound used to modulate, e.g., selectively inhibit, the development of Th17 T-cells from naïve precursors is a compound according to formula I:

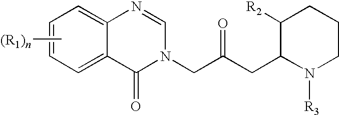

I, or a salt, isomer, derivative, precursor, analog, solvate enantiomer, diasteriomer and/or multimer thereof, where R₁ is hydrogen, halogen, nitro, benzo, lower alkyl, phenyl or lower alkoxy; R₂ is hydroxy, acetoxy, or lower alkoxy, R₃ is hydrogen lower alkoxy-carbonyl or lower alkenoxy-carbonyl, and n is 1, 2, 3 or 4. The compound is administered in an amount effective to modulate the development of Th17 T-cells from naïve precursors in a subject. For example, the compound is febrifugine, a precursor thereof, or a derivative thereof. Or, the compound is halofuginone, a precursor thereof, or a derivative thereof. A precursor compound includes a prodrug that is administered in an inactive form and processed by the recipient or by exposure to a physical condition, e.g., light and/or heat, or by exposure to a chemical entity to yield an active form of the drug.

Halofuginone modulates Th17 differentiation by stimulating an amino acid starvation response (AAR), which results from the intracellular accumulation of uncharged transfer RNAs (tRNAs). The invention also includes methods for stimulating the AAR using other compounds that cause accumulation of uncharged tRNAs. Examples of such compounds, including, for example, compounds that are structurally unrelated to halofuginone, include histidinol (an inhibitor of histidinyl tRNA charging) (Hansen et al., Reversible inhibition by histidinol of protein synthesis in human cells at the activation of histidine. Biol. Chem. Vol. 247(12):3854-7 (1972)), tryptophanol (an inhibitor of tryptophanyl tRNA charging) (Paley et al., Tryptophanyl-tRNA synthetase in cell lines resistant to tryptophan analogs. Exp. Cell Res. vol. 195(1):66-78 (1991)) and reveromycin (an inhibitor of isoleucyl tRNA synthetase) (Miyamoto et al., Identification of Saccharomyces cerevisiae isoleucyl-tRNA synthetase as a target of the G1-specific inhibitor Reveromycin A., J Biol. Chem. vol.; 277(32):28810-4. Epub 2002).

The invention also provides methods for inducing an amino acid starvation response (AAR) in a subject in need thereof, the method comprising administering to the patient a compound selected from a compound that selectively inhibits the development of Th17 T cells or a compound that induces intracellular accumulation of uncharged transfer ribonucleic acids (tRNAs), wherein the compound is administered in an amount effective to induce AAR in the subject.

For example, the compound is a compound of formula I:

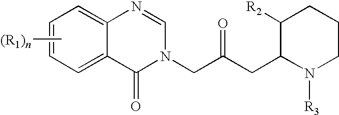

I, or a salt, isomer, derivative, analog, solvate, enantiomer, diasteriomer or multimer thereof, wherein: R₁ is selected from hydrogen, halogen, nitro, benzo, lower alkyl, phenyl and lower alkoxy; R₂ is selected from hydroxy, acetoxy, and lower alkoxy; R₃ is selected from hydrogen lower alkoxy-carbonyl and lower alkenoxy-carbonyl, and n is selected from 1, 2, 3 and 4; in an amount effective to effective to AAR in a subject.

For example, the compound is febrifugine, halofuginone, histidinol, tryptophanol, reveromycin, or a derivative thereof. In some embodiments, the compound is formulated for systemic administration. In some embodiments, the compound is formulated as an injectable composition.

The compositions of the invention also include a selective Th17 inhibitor that binds one or more molecular targets for halofuginone (HF), or otherwise interferes with the binding of halofuginone and one or more molecular targets for halofuginone. In one embodiment, the selective Th17 inhibitor is a multimer that includes two or more subunits linked together to produce a small molecule inhibitor of the development and/or expansion of Th17 cells. In one embodiment, the selective Th17 inhibitor is a multimer that comprises two or more subunits of halofuginone (HF) or a derivative of HF. The multimers provided herein are homomultimers or heteromultimers. As used herein, the term “homomultimer” refers to a multimer in which each subunit is the same. As used herein, the term “heteromultimer” refers to a multimer that contains at least two different derivatives of the same subunit or a multimer that contains at least two different types of subunits.

In the multimers provided herein, each subunit can be a small molecule inhibitor of the development and/or expansion of Th17 cells individually, such that when the subunits are linked together, the multimer exhibits the same or greater ability to inhibit the development and/or expansion of Th17 cells. For example, the linking of subunits to produce a multimer can exhibit a cumulative effect in which the ability of the multimer to inhibit the development and/or expansion of Th17 cells is greater than the ability exhibited by any one subunit individually. The multimers of the invention can exhibit a synergistic effect. Alternatively, each subunit need not be able to inhibit the development and/or expansion of Th17 cells individually, provided that, when the subunits are linked to form a multimer, the resulting multimer is able to inhibit the development and/or expansion of Th17 cells.

In the multimers provided herein, the subunits are linked. Suitable linkers for use in the multimers of the invention include, but are not limited to alkyl, alkene, alkyne, ether, ester, or amide linkages; carbon-nitrogen, carbon-sulfur linkages, and any chain using combinations of these linkages. In some embodiments, the linker or linkers are substituted at one or more positions in the main linker chain to modify linker flexibility, stability or hydrophilicity, including, e.g., substitution with hydroxy, keto, acetoxy, alkoxy, phenyl, phenoxy, amino, halogen, or nitro groups. A preferred mode of linking would be through the R1 positions of each subunit of the multimer, for example, by using an alkynyl linkage as shown in the dimer of FIG. 8, as it has been shown that this linkage does not interfere with HF activity. The multimers provided herein can contain any number of subunits. For example, multimers of the invention include dimers, trimers, tetramers, pentamers, hexamers. Preferably, the number of subunits in the multimer is between 2 and 30.

The subunits of the multimers provided herein can be a compound according to formula I described above or a salt, isomer, derivative, analog, solvate enantiomer, and/or diasteriomer thereof.

The compound is administered in an amount effective to modulate the development and/or expansion of Th17 cells in a subject. For example, the compound is febrifugine, or a derivative thereof. Or, the compound is halofuginone, or a derivative thereof.

The dimeric HF derivatives synthesized with linkers bind targets of HF with much higher avidity (an increased effective affinity owing to multiplicity of binding sites) than HF alone. This increased avidity of target binding increases potency. For example the increased avidity of target binding increases potency by at least 10-100 fold. Adjustments to the hydrophobicity and flexibility of the linker are made to optimize solubility of the HF derivatives, as well as their cell permeability and tissue penetration, thereby generating therapeutic compounds with optimal bioactivity.

In various aspects, the compound used for decreasing the appearance of cellulite in a subject inhibits maturation of myofibroblasts. Such compounds are referred to herein as “myofibroblast maturation inhibitors” or “myofibroblast maturation inhibitory compounds”. For example, the myofibroblast maturation inhibitory compound used for decreasing the appearance of cellulite in a subject compound inhibits one or more biological activities of myofibroblasts, such as expression of actin-containing stress fibers, expression and organization of fibronectin into fibrils, and formation of large fibronexus adhesion complexes.

In various aspects, the compound used for decreasing the appearance of cellulite in a subject modulates extracellular matrix (ECM) remodeling. These compounds are referred to herein as “ECM remodeling compounds”. It should be noted that the terms “myofibroblast maturation inhibitory compound” and “ECM remodeling compound” are not mutually exclusive—a compound of the invention that modulates the formation and/or progression of cellulite in a subject can be both a myofibroblast maturation inhibitory compound and an ECM remodeling compound.

The myofibroblast maturation inhibitory compounds, ECM remodeling compounds and myofibroblast maturation inhibitory/ECM remodeling compounds of the invention include compounds that are structurally related to the compound of Formula I, and in particular, compounds that are structurally related to and/or derived from halofuginone. Alternatively, myofibroblast maturation inhibitory compounds, ECM remodeling compounds and myofibroblast maturation inhibitory/ECM remodeling compounds of the invention include compounds that are structurally unrelated to halofuginone, but exhibit similar inhibitory effects on myofibroblast differentiation and extracellular matrix remodeling. By way of non-limiting examples, suitable myofibroblast maturation inhibitory compounds, ECM remodeling compounds and myofibroblast maturation inhibitory/ECM remodeling compounds of the invention include agonists of the peroxisome proliferator activator receptor gamma (PPAR-gamma), which have inhibitory effects on myofibroblast differentiation and fibrosis (Burgess, et al., Am J Physiol Lung Cell Mol Physiol, vol. 288:L1146-53 (2005); Wang, et al., Mol Endocrinol, vol. 14:1550-6 (2000)). Such compounds include ligands of the rosiglitazone family of PPARgamma agonists, triterpenoid PPARgamma agonists, FMOC-L-Leucine (see e.g., Rocchi, et al., Mol Cell 8:737-47 (2001); Wang, et al., Mol Endocrinol, vol. 14:1550-6 (2000); and Burgess, et al., Am J Physiol Lung Cell Mol Physiol, vol. 288:L1146-53 (2005)). Of these classes of agonists, FMOC-L-Leucine is a preferred example because it lacks the undesirable side effect of pro-adipogenic activity associated with other PPARgamma agonists (Rocchi, et al., Mol Cell 8:737-47 (2001)).

The compounds of the invention are formulated for topical administration, for example, as a film, membrane, foam, gel, or cream.

The invention also features a method of screening for inhibitors of cellulite formation, by administering a test compound to a tissue test area in a subject exhibiting cellulite; measuring the contractile force exerted by vertical collagen fibers tethered between fascia and dermo-hypodermal interface within the tissue test site; and comparing the contractile force measured in the tissue test area with a contractile force exerted by vertical collagen fibers in a second tissue site exhibiting cellulite in the subject, where a decrease in the contractile force in the tissue test area indicates that the test compound is an inhibitor of cellulite formation.

The invention also features a method of identifying an inhibitor of the formation and/or progression of cellulite and other skin disorders by contacting a cell expressing Rubvbl2 with a composition including halofuginone, a precursor thereof, or a derivative thereof, under conditions sufficient to allow binding, contacting the cell with a test compound under conditions sufficient to allow binding; and determining whether the test compound competes with halofuginone, a precursor thereof, or a derivative thereof, for binding of Rubvbl2.

The invention also features a method of identifying a putative inhibitor of cellulite formation by culturing a control population of primary dermal fibroblasts in a matrix of Type I collagen under conditions that allow the fibroblasts to attach to the substratum and spread, culturing a second population of primary dermal fibroblasts in a matrix of Type I collagen under the same conditions as the first (control) population of fibroblasts, except for the presence of a test compound, and comparing the ability of the populations of fibroblasts to attach to the substratum and spread, whereby failure of the test population of fibroblasts to attach and spread indicates that the test compound is a putative inhibitor of cellulite formation.

### BRIEF DESCRIPTION OF THE FIGURES

FIG. 1A is an illustration depicting the chemical structure of halofuginone (HF). Potential sites for chemical derivatization are indicated by the numbers 1-4. FIG. 1B is an illustration depicting the structure of the inactive HF derivative MAZ1310. FIG. 1C is an illustration depicting the molecular structure of the type 1 TGFÿ receptor kinase inhibitor SB-431542.

FIG. 2 is an illustration depicting the reciprocal development of Treg and Th17 cells. TGFβ in the presence or absence of IL-6 regulates a critical decision (shown in the box) between the autoimmune effector Th17 cells (marked by expression of IL17) and the regulatory Treg cells (marked by expression of FoxP3).

FIGS. 3A, 3B and 3C are a series of graphs depicting the non-specific cytoxicity of HF in both normal and transformed T-cells at high doses. Primary T-cells (FIG. 3A) or the transformed T cell line Jurkat (FIG. 3B) were treated with HF (100 nM unless otherwise indicated), and tested for apoptosis. At 100 nM HF, but not 30 nM or lower, generalized T-cell apoptosis is observed (FIG. 3C).

FIG. 4 is a series of graphs depicting the ability of low doses of HF to enhance Treg differentiation while suppressing Th17 differentiation. 2.2-10 nM HF increases expression of FoxP3, a Treg marker in TGFβ/IL-6 treated T-cells concomitantly with inhibiting expression of IL17, a marker of Th17 differentiation.

FIG. 5 is a graph depicting the ability of low doses of HF to enhance Treg differentiation while suppressing Th17 differentiation. The data shown in this bar graph representation was derived from experiments similar to those shown in FIG. 4.

FIG. 6 is a graph depicting the non-specific effects of HF on B and T cells at high doses. At doses greater than 20 nM, a variety of effects on B and T cell proliferation and differentiation were seen in response to HF treatment.

FIG. 7 is a graph depicting that the effects of HF on Th17 differentiation were seen at ˜10 fold lower doses than the doses at which generalized effects on T-cell proliferation and differentiation were observed.

FIG. 8 is an illustration depicting the structure of a predicted active dimeric halofuginone derivative (top) and a predicted inactive variant (bottom).

FIG. 9 is an illustration depicting a general scheme for synthesis of dimeric halofuginone derivatives.

FIGS. 10A-10G are a series of illustrations and graphs depicting the selective inhibition of Th17 cell development by halofuginone.

FIG. 10A (left panel) is a graph depicting dose-response analyses on activated CFSE-labeled CD4⁺ CD25⁻ T cells in the presence of DMSO, 40 nM MAZ1310 or titrating concentrations of HF (1.25-40 nM). CFSE dilution and cell-surface CD25 expression were determined 48 hours after activation. Intracellular cytokine production was determined on day 4 or 5 following a 4 hour restimulation with PMA and ionomycin in the presence of brefeldin A. CFSE dilution and percentages of cells expressing CD25, IFNγ⁺ IL4⁻ (Th1 cells), IL-4⁺ IFNγ⁻ (Th2 cells) or IL-17⁺ IFNγ⁻ (Th17 cells) cells are displayed and the values are normalized to T cells treated with 40 nM MAZ1310±SD. FIG. 10A (right panel) is a graph depicting dose-response analyses of HF effects on CD8⁺ T cell or B cell function. T or B cells were activated as described in the materials and methods in the presence of DMSO, 40 nM MAZ1310 or titrating concentrations of HF (1.25-40 nM). CFSE dilution, cell-surface CD25 expression and intracellular cytokine production was determined as above 2-5 days after activation. CFSE dilution and percentages of CD8⁺ T cells expressing CD25, IFNγ⁺ granzyme B⁺ (cytotoxic T lymphocytes) or IL-6⁺ B cells are displayed, and the values are normalized to cells treated with 40 nM MAZ1310±SD.

FIG. 10B is a table depicting the IC₅₀ values calculated for the effects of HF on CD4⁺ CD25⁻ T cell functions as indicated.

FIG. 10C is a graph depicting the effect of the racemic mix of HF (HF) or HPLC-purified D- or L-enantiomers of HF (HF-D, or HF-L) on CD4⁺ CD25⁻ T cells activated in the presence of TGFβ plus IL-6. The percent of Th17 cells (IL-17⁺ IFNγ⁻) was determined by intracellular cytokine staining on day 4 and values are normalized to cells treated with 40 nM MAZ1310±SD.

FIG. 10D is a graph depicting the effect of HF on CD4⁺ CD25⁻ T cells activated in the indicated cytokine conditions when 10 nM HF was added at the indicated times following activation. The percent of Th17 cells (IL-17⁺ IFNγ⁻) was determined by intracellular staining 4 days after activation as above and values are presented as mean percent of Th17 cells±SD. Asterisks indicate statistical significance (p<0.005) relative to T cells treated with 10 nM MAZ1310 at the time of activation.

FIG. 10E is an illustration depicting CFSE-labeled T cells activated in the indicated cytokine conditions in the presence of DMSO, 5 nM HF, 10 nM HF, 10 nM MAZ1310 or 10 μM SB-431542. Foxp3 intracellular staining was performed 3 days after T cell activation and intracellular cytokine staining was performed on day 4 as above.

FIG. 10F is an illustration depicting purified primary human memory T cells (CD4⁺ CD45RO⁺) activated by anti-CD3/anti-CD28 coated beads in co-culture with CD14⁺ monocytes and treated with DMSO, 100 nM HF or 100 nM MAZ1310. T cells were expanded for 6 days and intracellular cytokine expression was determined following restimulation with PMA plus ionomycin in the presence of brefeldin A.

FIG. 10G is a graph depicting the percent of IL-17⁻ (black bars) or IFNγ⁻ (white bars) expressing T cells upon treatment with the indicated additives. The data were normalized to T cells treated with MAZ1310 and are displayed as mean values±SD. Asterisk indicates statistical significance (p<0.05). All data represent at least 3 similar experiments.

FIGS. 11A-11E are a series of graphs and illustrations demonstrating that HF-dependent inhibition of Th17 differentiation is mediated by STAT3.

FIG. 11A is a series of graphs depicting representative histograms displaying the kinetics of STAT3 phosphorylation in developing Th17 cells treated with or without HF. Resting naïve T cells (shaded peak), T cells activated in the presence of TGFβ plus IL-6 (TGFβ/IL-6) treated with 10 nM MAZ1310, TGFβ/IL-6-activated T cells treated with 5 nM HF, TGFβ/IL-6-activated T cells treated with 10 nM HF. T cells were fixed at the indicated times and intracellular phospho-STAT3 staining was performed.

FIG. 11B is an illustration depicting CD4⁺ CD25⁻ T cells treated with 10 nM HF or 10 nM MAZ1310 and activated in the presence or absence of TGFβ plus IL-6. Whole cell lysates were generated at the indicated times following activation, and western blotting was performed using the indicated antibodies.

FIG. 11C is an illustration depicting CD4⁺ CD25⁻ T cells from YFP^{fl/fl} or STAT3C-GFP^{fl/fl} mice treated with recombinant TAT-Cre, wherein the cells were activated in the presence or absence of TGFβ plus IL-6 and treated with DMSO, 5 nM HF, 10 nM HF or 10 nM MAZ1310 as indicated. Activated T cells were restimulated after 4 days and intracellular cytokine staining was performed as in FIG. 10A. T cells expressing YFP or GFP are gated on as shown.

FIG. 11D is a graph displaying the percent of Th17 cells (IL-17⁺ IFNγ⁻) within YFP⁻ cells (black bars), YFP⁺ cells (grey bars), STAT3C-GFP⁻ cells (white bars) or STAT3C-GFP⁺ (etched bars). The data are normalized to DMSO-treated cultures and are presented as mean values±SD on duplicate samples. Asterisks indicate statistical differences between STAT3C-GFP⁺ cells and YFP⁺ cells (p<0.05).

FIG. 11E is an illustration depicting CD4⁺ CD25⁻ T cells activated in medium or TGFβ plus IL-6 and treated with DMSO, 10 nM HF, 10 nM MAZ1310, or 10 nM HF plus 10 μM SB-431542. Foxp3 expression was determined on day 3 by intracellular staining. All experiments were performed at least 3 times with similar results.

FIGS. 12A-12F are a series of graphs and illustrations demonstrating that HF induces an amino acid response in T cells.

FIG. 12A is an illustration depicting dot plot analyses of microarray data from CD4⁺ CD25⁻ T cells treated with 10 nM HF or 10 nM MAZ1310 activated in Th17 polarizing cytokine conditions for 3 or 6 hours. The lighter dots in the upper right quadrant indicate transcripts increased at least 2-fold by HF treatment at both 3 and 6 hours. Hallmark amino acid starvation response genes are identified by text and arrowheads.

FIG. 12B is an illustration depicting dot plot analyses of gene expression in T cells treated for 6 hours with either 10 nM HF or MAZ1310. Chi-squared analysis shows the expression distribution of genes previously found to be regulated by ATF4 in tunicamycin-treated mouse embryonic fibroblasts (darker dots).

FIG. 12C is a graph depicting the results of quantitative real-time PCR performed on cDNA generated from resting naïve T cells (T_{N}) or T cells activated for 4 hours in the presence of 10 nM MAZ1310 or 10 nM HF. Asns, Gpt2 or eIF4Ebp1 mRNA expression was normalized to Hprt levels and data are presented as mean values±SD in duplicate samples.

FIG. 12D is an illustration depicting immunoblot analysis of purified CD4⁺ CD25⁻ T cells that were either unstimulated, or TCR-activated without exogenous cytokines in the presence of DMSO, 40 nM MAZ1310 or titrating concentrations of HF (1.25-40 nM). Whole cell lysates were prepared 4 hours-post TCR activation and immunoblotting was performed with the indicated antibodies. ATF4 protein is indicated by arrowhead. NS—non-specific band.

FIG. 12E is an illustration depicting immunoblot analysis of purified CD4⁺ CD25⁻ T cells activated through the TCR for the indicated times without exogenous cytokines in the presence of either 10 nM MAZ1310 or 10 nM HF as indicated. Whole cells lysates were prepared during the timecourse and immunoblotting was performed.

FIG. 12F is an illustration depicting immunoblot analysis of CD4⁺ CD25⁻ T cells that were either left unstimulated or were TCR-activated in the absence or presence of the indicated polarizing cytokine conditions. Cultures were further supplemented with either 10 nM MAZ1310 or 10 nM HF as indicated. Whole cell lysates were generated 4 hours after activation and immunoblotting was performed. Microarray data were generated from triplicate samples and all other data are representative of at least 2 similar experiments.

FIGS. 13A-13F are a series of graphs and illustrations demonstrating that amino starvation-induced stress response in T cells inhibits Th17 differentiation.

FIG. 13A is an illustration depicting analysis of CD4⁺ CD25⁻ T cells that were left unstimulated (T_{N}), or were activated through the TCR for 4 hours in complete medium (complete—200 μM Cys/100 μM Met), medium lacking Cys/Met (⁻Cys/Met) or complete medium containing 1 μg/ml tunicamycin, 10 nM HF or 10 nM MAZ1310. Western blotting was performed on whole cell extracts with the indicated antibodies. Xbp-1 splicing assay was performed on cDNA synthesized from T cell cultures.

FIG. 13B is a graph depicting dose-response analyses of L-cysteine/L-methionine (Cys/Met) concentrations on T cell activation and differentiation. Activated CD4⁺ CD25⁻ T cells were cultured in the absence or presence of polarizing cytokines to induce Th1, Th2, iTreg or Th17 differentiation in titrating concentrations of Cys/Met as indicated. CD25 and Foxp3 expression was determined on day 3, cytokine production determined by intracellular staining on day 4 or 5 as in FIG. 10A. Percentages of cells expressing CD25, Foxp3, IFNγ+IL4⁻ (Th1 cells), IL-4⁺ IFNγ⁻ (Th2 cells) or IL-17⁺ IFNγ⁻ (Th17 cells) cells are displayed and the values are normalized to T cells cultured in complete medium (200 μM Cys/100 μM Met).

FIG. 13C is an illustration depicting analysis of T cells cultured in complete medium (complete—200 μM Cys/100 μM Met/4 mM Leucine), medium containing 0.1× cysteine and methionine (Cys/Met), medium containing 0.1× leucine (Leu) or complete medium plus 0.2 mM L-tryptophanol. Cells were activated in the presence or absence of TGFβ plus IL-6 as indicated, expanded in for 4 days and restimulated with PMA and ionomycin for intracellular cytokine staining.

FIG. 13D is a graph depicting representative histograms showing the kinetics of STAT3 phosphorylation in CD4⁺ CD25⁻ T cells activated in the presence of TGFβ plus IL-6. Resting naïve T cells (grey, shaded peak), T cells cultured in complete medium (200 μM Cys/100 μM Met), T cells cultured in low Cys/Met concentrations (10 μM Cys/5 μM Met), T cells cultured in complete medium with 10 nM HF. T cells were fixed at the indicated times and intracellular phospho-STAT3 staining was performed as in FIG. 11A.

FIG. 13E is an illustration depicting quantification of the intracellular phospho-STAT3 data shown in FIG. 13C. Data are presented as the percent of phospho-STAT3⁺ T cells in each condition multiplied by mean fluorescence intensity (MFI). Mean values from duplicate samples are displayed±SD. All data represent 2-3 similar experiments.

FIG. 13F is a graph depicting analysis of CD4⁺ CD25⁻ T cells cultured in the presence of titrating concentrations of tunicamycin as indicated. These cells were analyzed for CD25 upregulation or differentiation into Th1, Th2, iTreg or Th17 cells as described in FIGS. 10A and 13B.

FIGS. 14A-14C are a series of graphs and illustrations depicting the effects of HF treatment on T cell activation and effector function.

FIG. 14A is an illustration depicting the analysis of CFSE-labeled CD4⁺ CD25⁻ T cells treated with DMSO, 5 nM HF or 5 nM MAZ1310 and activated in the absence of exogenous cytokines. CFSE dilution and CD25 cell surface expression was determined on day 2 by FACS analyses. T cells were activated as above without exogenous cytokines and supernatants were harvested at the indicated time-points following activation. Cytokine secretion was determined using a cytometric bead array (CBA) on duplicate samples. Cytokine concentrations were determined by comparison to standard curves and data are presented as the mean cytokine concentrations±SD.

FIG. 14B is an illustration and a graph depicting the analysis of CFSE-labeled CD4⁺ CD25⁻ T cells that were activated under the following conditions: Th “null” (ThN)=no exogenous cytokines, Th1=IL-12 plus anti-IL-4, Th2=IL-4 plus anti-IFNγ, iTreg=TGFβ, Th17=TGFβ plus IL-6. DMSO, 5 nM HF or 5 nM MAZ1310 was added to the cells at the time of T cell activation as indicated. Intracellular Foxp3 staining was performed on expanded cells 3 days after activation. Cytokine expression was determined by intracellular staining after re-stimulation with PMA and ionomycin for 4 hours in the presence of brefeldin A. These data are representative of at least 3 independent experiments.

FIG. 14C is a graph depicting HF effects on Il17 and Il17f mRNA expression in Th17 cells. CD4⁺ CD25⁻ T cells were differentiated under Th17 cytokine conditions in the presence of DMSO, 10 nM HF or 10 nM MAZ1310 for 4 days as above. Cells were harvested, restimulated with PMA and ionomycin as above and cDNA was generated for Sybrgreen real-time PCR analysis. Data indicate fold changes in mRNA expression normalized to HPRT and are presented as mean expression±SD. Asterisks indicate statistical significance for Il17 mRNA (p<0.001) and Il17f mRNA (p<0.05) for HF-treated T cells relative to those treated with MAZ1310.

FIGS. 15A-15D are a series of illustrations demonstrating that HF does not regulate TGFβ signaling in T and B cells.

FIG. 15A is an illustration depicting the analysis of CD4⁺ CD25⁻ T cells that were activated in Th1 or Th2 polarizing conditions as described in FIG. 14, either in the presence or absence of TGFβ. DMSO, 10 nM HF, 10 nM MAZ1310 or 10 μM SB-431542 was added as indicated at the time of activation and intracellular cytokine staining was performed on expanded T cells on day 5 as in FIG. 14B

FIG. 15B is an illustration depicting the analysis of CD8⁺ T cells that were activated in the presence or absence of TGFβ and cultured with DMSO, 10 nM HF, 10 nM MAZ1310 or 10 μM SB-431542. Expanded cells were restimulated on day 5 and intracellular staining was performed as above.

FIG. 15C is an illustration depicting the analysis of CFSE-labeled B cells that were activated by LPS stimulation in the presence or absence of TGFβ plus DMSO, 10 nM HF, 10 nM MAZ1310 or 10 μM SB-431542. Intracellular IL-6 production in B cells re-stimulated with PMA plus ionomycin, or cell-surface IgA expression was determined 4 days after activation by FACS analyses.

FIG. 15D is an illustration depicting the analysis of purified CD4⁺ CD25⁻ T cells that were treated with DMSO, 40 nM MAZ1310, titrating concentrations of HF (2.5-40 nM) or 10 μM SB-431542 for 30 minutes in complete medium supplemented with 0.1% fetal calf serum. T cells were then activated in the presence or absence of 3 ng/ml TGFβ. Whole cell extracts were prepared after 1 hour of stimulation and western blot analyses were performed using the indicated antibodies. These data are representative of 3 similar experiments.

FIGS. 16A-16C are series of graphs and illustrations demonstrating that HF inhibits RORγt function, but not expression.

FIG. 16A is a graph depicting the analysis of CD4⁺ CD25⁻ T cells that were treated with DMSO (if no indication), 10 nM HF or 10 nM MAZ1310 and were activated in the presence of the indicated cytokines. T cells were harvested at the indicated times following activation, RNA was isolated and quantitative real-time PCR was performed using RORγt-specific primers and taqman probe. RORγt expression was normalized to Gapdh levels, and the data are presented as fold changes relative to naïve T cells.

FIG. 16B is an illustration depicting the analysis of CD4⁺ CD25⁻ T cells that were activated in the presence or absence of TGFβ plus IL-6 and were transduced with empty (MIG) or RORγt-expressing (MIG.RORγt) retroviruses 12 hours-post activation. Infected T cells were expanded and restimulated on day 4 for intracellular staining. MIG- and MIG.RORγt-transduced cells were gated based on GFP fluorescence.

FIG. 16C is a graph depicting the percent of Th17 cells (IL-17⁺ IFNγ⁻) in cultures of MIG-transduced (black bars) or MIG.RORγt-transduced (white bars) T cells as determined by intracellular cytokine staining were normalized to DMSO-treated cultures. The data are presented as mean values±SD on duplicate samples. These data are representative of 3 similar experiments.

FIGS. 17A-17B are a series of illustrations demonstrating that HF-enforced Foxp3 expression is not necessary or sufficient for the inhibition of Th17 differentiation.

FIG. 17A is an illustration depicting the analysis of CD4⁺ CD25⁻ T cells that were activated in the presence or absence of TGFβ plus IL-6 and were transduced with empty (pRV) or FOXP3-expressing (pRV.FOXP3) retroviruses 12 hours after activation. Intracellular FOXP3 and cytokine expression was determined 3 days after infection (4 days after activation). IFNγ and IL-17 expression in pRV- and pRV.FOXP3-transduced cells was determined by gating on GFP⁺ cells.

FIG. 17B is an illustration depicting FACS sorted naïve CD4⁺ T cells from wild-type (WT) or Foxp3-deficient (Foxp3 KO) male mice that were treated with DMSO, 10 nM HF or 10 nM MAZ1310 as indicated and activated in the absence or presence of TGFβ plus IL-6. T cells were expanded and were re-stimulated on day 4 for intracellular cytokine staining. These results are representative of cells purified from 2 pairs of WT and Foxp3 KO mice.

FIG. 18 is an illustration demonstrating that HF induces a stress response in fibroblasts. SV-MES mesangial cells were stimulated for 2 hours with DMSO, 20 nM MAZ1310 or 20 nM HF. Whole cell lysates were analyzed for expression of phosphorylated or total eIF2α or GCN2 by western blotting. These data represent at least 2 similar experiments.

FIGS. 19A-19D are a series of graphs and illustrations demonstrating that amino acid deprivation mimics the effects of HF on T cell differentiation.

FIG. 19A is an illustration depicting the analysis of CD4⁺ CD25⁻ T cells that were activated through the TCR for the indicated times without polarizing cytokines in the presence or absence of L-cysteine and L-methionine. Whole cell lysates were prepared and immunoblotting was performed using the indicated antibodies.

FIG. 19B is a graph depicting the results of quantitative real-time PCR performed on cDNA generated from naïve T cells, either left unstimulated or activated through the TCR for 4 hours without exogenous cytokines in the presence or absence of cysteine and methionine (Cys/Met) as indicated. Asns, Gpt2 or eIF4Ebp1 mRNA expression was normalized to Hprt levels and data are presented as mean values±SD in duplicate samples.

FIG. 19C is an illustration depicting the analysis of CD4⁺ CD25⁻ T cells that were cultured in either complete medium (200 μM Cys/100 μM Met) or medium containing limiting concentrations of amino acids (20 μM Cys/10 μM Met). These cells were activated through the TCR in the absence or presence of polarizing cytokines to induce Th1, Th2, iTreg or Th17 differentiation. Foxp3 intracellular staining was performed on day 3-post activation and intracellular cytokine expression was determined on cells re-stimulated with PMA plus ionomycin after 4-5 days.

FIG. 19D is an illustration depicting the analysis of CD4⁺ CD25⁻ T cells that were labeled with CFSE, cultured in medium containing the indicated concentrations of cysteine and methionine (Cys/Met) and activated in the absence or presence of TGFβ plus IL-6. Cells were expanded until day 4 when CFSE dilution and intracellular cytokine production was determined on cells re-stimulated with PMA and ionomycin. Cells with equivalent CFSE fluorescence are gated on as indicated and intracellular cytokine expression is shown within each gated population.

FIGS. 20A-20D are a series of graphs and illustrations demonstrating in vivo activation of the AAR pathway by HF.

FIG. 20A is a graph depicting splenocytes from control- and HF-treated mice. C57B/6 mice were injected i.p. with vehicle (DMSO) or 2.5 μg HF. Spleens were harvested 6 hours post injection, red blood cells were lysed and total splenocytes were counted. Data are presented as mean cell counts±SD from 2 mice per group.

FIG. 20B is an illustration depicting FACS analyses of splenocytes from mice injected with DMSO or 2.5 μg HF as in FIG. 20A.

FIG. 20C is a graph depicting eIF2α expression in control- and HF-treated mice. Splenocytes from mice injected with DMSO or HF as above were harvested 6 hours post injection. Red blood cells were lysed and immunoblotting was performed on whole cell extracts for phosphorylated or total eIF2α as indicated.

FIG. 20D is a graph depicting the results of quantitative real-time PCR (qPCR) experiments performed for AAR-associated gene expression (Asns, Gpt2, eIF4Ebp1) using cDNA generated from splenocytes of mice injected with DMSO or HF as above. Expression of AAR-associated genes were normalized to Hprt levels and data are presented as mean relative expression from duplicate samples±SD.

### DETAILED DESCRIPTION OF THE INVENTION

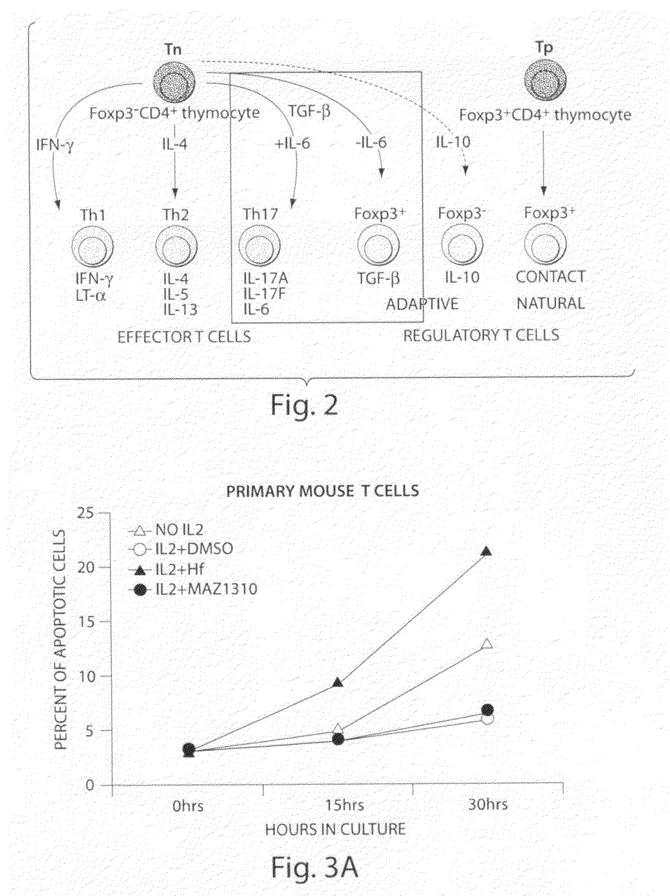

The present invention provides methods and compositions for modulating, e.g., reducing, inhibiting, or preventing, the development and/or expansion of Th17 T-cells from naïve precursors in a subject, particularly for the treatment of cellulite. Th17 cells are a subset of effector T-cells that have a role in mediating autoimmune responses. Naïve T-cells can differentiate in response to stimuli into a variety of effector T-cells with distinct roles in both host defense and autoimmune pathogenesis. CD4 effector T-cells were originally subdivided into two distinct classes, Th1 and Th2. However, a third effector T-cell lineage, Th17, has been identified. Originally characterized as a CD4 lineage stimulated by the causative agent of Lyme disease, Borrelia burgdorferi, Th17 cells were defined by expression of several genes that distinguished them from Th1 and Th2 cells, particularly the cytokine IL-17. T-helper cell differentiation into Th1, Th2, Th17 or Treg T cells is regulated by a variety of cytokines. Treg and Th17 cells develop through reciprocal interactions that utilize the dual characteristics of two cytokines, IL-6 and TGFbeta (FIG. 2). The synergistic action of these two cytokines stimulates the differentiation of activated naïve T-cells to IL-17 secreting Th17 cells.

TGFbeta has been shown to have reciprocal activities for the suppression or expansion of Th17 cells, depending on the cytokine environment. TGFbeta can differentiate naïve T-cells into regulatory T-cells (Tregs) that inhibit autoimmunity and protect tissues from cell-mediated damage. Alternatively, in the presence of the cytokine IL-6, TGFbeta signals the expansion of tissue damaging Th17 cells, using the transcription factor RORgammaT. Furthermore, this mechanism is a switch: inhibition of Th17 cells is sufficient to cause the expansion of tissue-protective Tregs, and vice versa.

The methods and compositions of the invention include a selective Th17 inhibitor that modulates the development and/or expansion of Th17 cells for the treatment of cellulite. Selective Th17 inhibitors of the invention modulate the development and/or expansion of Th17 cells by specifically inhibiting, partially or completely, the development of naïve T cells into Th17 cells, such that the naïve cells are turned away from the Th17 lineage, which is associated with cell-mediated damage, persistent inflammation and auto-immunity. In some embodiments, the selective Th17 inhibitors modulate the reciprocal interactions involving Th17 cells and Treg cells. In these embodiments, the selective Th17 inhibitor alters the development of the naïve T cells away from the Th17 lineage and promotes or otherwise induces the developing T cells toward the Treg lineage, which is thought to be anti-inflammatory and tissue protective. The selective Th17 inhibitors of the invention modulate the development and/or expansion of Th17 cells by specifically inhibiting, reducing or otherwise impeding the ability of TGFbeta to promote the expansion of Th17 cells in IL-6.

The selective Th17 inhibitors provided herein exhibit a specific inhibitory effect on a specific class of T-cells, i.e., Th17 cells, as opposed to a generalized inhibition of T-cell activation or other generalized immunosuppression.

Cellulite

Cellulite is a skin disorder that is characterized by dimpling and puckering of the skin of the thighs and buttocks. It is rare in men and quite common in women, with the estimated frequency of 85-98% of post-pubertal females of all races being affected to some extent (Avram, M, J. Cosmet Laser Ther, vol. 6: 181-185 (2004)). Cellulite is a poorly understood and controversial topic; purported treatments abound, but are expensive. While different hypotheses exist to explain its etiology, there is no consensus opinion regarding the cause or progression of cellulite (See e.g., Avram, M, J. Cosmet Laser Ther, vol. 6: 181-185 (2004); Querleux, et al. Skin Res Technol 8, 118-24 (2002); Mirrashed, et al. Skin Res Technol 10, 161-8 (2004); and Smalls, J Cosmet Sci 56, 105-20 (2005)). Increased percentages of thigh fat are associated with a worsening of this condition. Cellulite is not simply a function of adipose volume, however, because very obese men rarely demonstrate cellulite and extremely slender women with good muscle tone can evidence marked dimpling (Rosenbaum, et al. Surg, vol. 101: 1934-39 (1998)). Even though cellulite is not a pathology, it poses a great cosmetic concern to a large number of people due to its prevalence. No effective strategy has been described to date for the prevention or long-term management of this condition.

Cellulite may be the result of gender differences in the biochemistry of either adipose tissue (fat) or connective tissue. While many popular treatments target the adipose component of this condition, there is no indication that adipose tissue in affected areas or individuals has an increased water content, or is in any way physiologically different from unaffected adipose tissue (Rosenbaum, et al. Surg, vol. 101: 1934-39 (1998); and Querleux, et al. Skin Res Technol 8, 118-24 (2002)). Microanatomical descriptions of cellulite vary significantly in the literature and often conflict with one another (Smalls, J Cosmet Sci 56, 105-20 (2005); Pierard, et al. Am J Dermatopathol 22, 34-7 (2000)); Rosenbaum, et al. Surg, vol. 101: 1934-39 (1998); Querleux, et al. Skin Res Technol 8, 118-24 (2002); and Mirrashed, et al. Skin Res Technol 10, 161-8 (2004)). Studies include histologic examination of samples (from autopsy specimens and/or live biopsy material, containing small numbers of dubiously matched controls), high-resolution magnetic resonance imaging (MRI), and sonography of skin regions affected by cellulite (Pierard, et al. Am J Dermatopathol 22, 34-7 (2000); Rosenbaum, et al. Surg, vol. 101: 1934-39 (1998); Querleux, et al. Skin Res Technol 8, 118-24 (2002); and Mirrashed, et al. Skin Res Technol 10, 161-8 (2004)).

Studies have demonstrated the existence of microanantomical differences between men and women at the connective tissue border that separates the dermis from the subcutaneous fat-containing hypodermis of thighs and buttocks (Pierard, et al. Am J Dermatopathol 22, 34-7 (2000); Rosenbaum, et al. Surg, vol. 101: 1934-39 (1998)). These differences underlie the sexual dimorphism seen in the presentation of the cellulite condition. While men (and a small percentage of women) have a smooth, continuous dermo-hypodermal interface and are unaffected by cellulite, the majority of women have discontinuities in this region that are accompanied by varying degrees of protrusion of adipose tissue into the dermis. Increased protrusion of fat into the dermis is eventually associated with the appearance and worsening of the condition cellulite. In women, and in the rarely affected men, this structural discontinuity is thought to develop at puberty and to be determined hormonally (Nurnberger, et al. J Dermatol Surg Oncol 4, 221-9 (1978); Rosenbaum, et al. Surg, vol. 101: 1934-39 (1998)). Regardless of whether female subjects currently are affected by cellulite, the vast majority of them possess these gender-linked discontinuities in the dermo-hypodermal interface, along with mild protrusion of fat into the dermis (Pierard, et al. Am J Dermatopathol 22, 34-7 (2000)). Furthermore, female subjects who are affected by cellulite demonstrate this pattern of connective tissue irregularity diffusely, both in affected and unaffected areas of the thigh (Rosenbaum, et al. Surg, vol. 101: 1934-39 (1998)). These observations indicate that, while the presence of connective tissue discontinuity at the dermo-hypodermal border is coincident with susceptibility to cellulite, this structural feature alone is insufficient to produce the skin dimpling that is the central defining characteristic of cellulite.

Structural differences between men and women in the connective tissues that tether the dermis to the superficial fascia (Mirrashed, et al. Skin Res Technol 10, 161-8 (2004); Pierard, et al. Am J Dermatopathol 22, 34-7 (2000); Querleux, et al. Skin Res Technol 8, 118-24 (2002); Rosenbaum, et al. Surg, vol. 101: 1934-39 (1998)). Connective tissue structural disparities between skin samples that either are, or are not, affected by cellulite also have been observed within the hypodermal space.

Model for Cellulite Formation and Progression

Gender differences in mechanical regulation of tensional homeostasis and dynamic tissue remodeling in the hypodermis result in skin dimpling. The hypodermis is the layer of subcutaneous fat that lies under the dermis, but above the fascia that surrounds muscle, and the periosteum that surrounds bone (Kanitakis, et al. Eur J Dermatol 12, 390-9; quiz 400-1 (2002)). In addition to fat lobules, the hypodermal space is composed of a vertically oriented 3-dimensional lattice network of fibrous elements connecting the reticular dermis with the superficial fascia. The hypodermal vertical collagen network is arranged in geometric subunits, allowing for skin mobility, and serving as an expansive-retractile reservoir for the deposition and absorption of fat. Connective tissue structures normally observed in this space include septae, composed of thick parallel collagen bundles, and smaller microfibrils, composed of collagen and elastin. Connective tissues are under mechanical tension even at rest, as evidenced by the fact that they do not, for the most part, sag. Maintenance of this mechanical load is called tensional homeostasis, and it must be re-established within the extracellular matrix after tissue injury, during repair. While tissues and ECM are stress-loaded, the resident cells of mature connective tissue are largely stress-shielded by the matrix that they deposit and remodel (Tomasek, et al., Nat Rev Mol Cell Biol 3, 349-63 (2002)). Fibroblasts that migrate during growth or in response to injury, however, become subject to mechanical forces, which can induce them to differentiate into myofibroblasts that possess contractile properties (Tomasek, et al., Nat Rev Mol Cell Biol 3, 349-63 (2002)). The myofibroblast phenotype is regulated by mechanical tension in vivo, as well as by the local release of some cytokines. Myofibroblasts are capable of reorganizing collagen networks along planes of mechanical tension, and can exert contractile force (Desmouliere, et al., Wound Repair Regen., vol. 13: 7-12 (2005); Hinz, et al. Thromb Haemost, vol 90: 993-1002 (2003); Hinz, et al., Curr Opin Biotechnol., vol. 14: 538-46 (2003); Tomasek, et al., Nat Rev Mol Cell Biol 3, 349-63 (2002)). Existing collagen fibers within the ECM also will align themselves along newly generated stress planes.

The model for cellulite formation, progression, and treatment described is consistent with observations reported in the literature and the data presented herein. While not intending to be bound by theory, it is believed that a majority of post-pubertal females of a given body mass index have a discontinuous dermo-hypodermal interface accompanied by regions of modest adipose protrusion. This discontinuous connective tissue border predisposes women to the eventual development of cellulite by destabilizing tensional homeostasis in the adipose compartment, or hypodermal space, of the thigh and buttock. While there are certainly structural differences between women, the predominant factor in the development of cellulite is a variable degree of response to the trigger of fat protrusion at the dermal adipose junction. In a women's thigh, the fragile, structurally compromised connective tissue border micro-anatomically “gives way” in response to a stimulus, such as the addition of fat to the area or regional mechanical stress, which can elicit varying degrees of response from a given tissue, in a given woman.

What follows is a dynamic process resulting in the formation and progression of cellulite, a process that is modulated, e.g., interrupted or reversed, pharmacologically. Local cytokine release occurs at the site of fat protrusion and connective tissue destruction, and these local cytokines initiate fibroblast migration in the vertical plane. Either cytokine release, or exposure to the mechanical forces of migration, triggers the differentiation/maturation of fibroblasts to myofibroblasts, as well as stimulates the increased deposition of ECM. The directed movement of fibroblasts generates a tractional force, thereby creating stress planes that initiate vertical realignment of existing hypodermal lattice structure—i.e., collagen microfibrils, and septae. The newly differentiated myofibroblasts express actin-containing stress fibers, express and organize fibronectin into fibrils, and form large fibronexus adhesion complexes that allow the generation of contractile force. Contractile myofibroblasts restore tensional homeostasis to the hypodermis through mechanical re-loading of the ECM. The net result of this process is to tether the dermis more tightly to the underlying fascia, creating skin dimples that are the characteristic sign of cellulite. Contractile force generation by collagen bundles tethered between the fascia and the dermo-hypodermal interface also leads to further protrusion of subcutaneous fat, and further destruction of the connective tissue border, creating a biomechanical feedback loop of fat protrusion, dimpling, and loss of elasticity within the hypodermal vertical lattice. Depending on the extent of response, myofibroblasts persist, and vertical collagen fibers thicken, becoming increasingly fibrotic. Microanatomical observations of cellulite-affected tissue (Pierard, et al. Am J Dermatopathol 22, 34-7 (2000); Querleux, et al. Skin Res Technol 8, 118-24 (2002); Rosenbaum, et al. Surg, vol. 101: 1934-39 (1998)) reflect this mechanism. In the extreme presentation, normal hypodermal connective tissue structures are remodeled entirely, and replaced with a tortuous, fibrotic network of collagen fibers. Consistent with all observations, this process is greatly exacerbated by increased fat deposition and losses of elasticity in the affected tissues.

A common perception in the art is that collagen-promoting factors are favorable to improving the appearance of cellulite, and that the connective tissue structures involved in cellulite formation are intractable. However, in one aspect of the current invention, dynamic tightening, and loss of elasticity in the connective tissue in the hypodermis is responsible for driving the protrusion of fat through the dermal-hypodermal interface. This process is treated using an ECM-remodeling compound, such as those described herein.

In the methods described herein, cellulite is treated with pharmacologic intervention, using small molecules that modify the behavior of fibroblasts and myofibroblasts in the dermis and hypodermis. The treatment model described herein is the only method, to date, that targets the formation of skin dimples in the thigh and buttock, by targeting the structures that define the condition cellulite. By using small molecule inhibitors to interfere with either the maturation or the action of myofibroblasts, dynamic processes that form the basis for cellulite appearance and progression—tissue remodeling of the hypodermal ECM, and progressive contractile force generation upon the dermo-hypodermal connective tissue interface—are modulated, e.g., prevented or reversed.

Stretch Marks

Stretch marks, also known as striae distensae, are a common disfiguring condition associated with continuous and progressive stretching of the skin. Stretch marks are commonly due to stretching of the skin, as in rapid weight gain, or mechanical stress, as in weight lifting.

Stretch marks are skin defects that look like bands, stripes, or lines. Stretch marks are seen when a person grows or gains weight rapidly or has certain diseases or conditions. Common causes for stretch marks include pregnancy, puberty, obesity, Cushing's syndrome (an endocrine disorder caused by high levels of cortisol in the blood), Iatrogenic Cushing syndrome, overuse of topical corticosteroid, and Ehlers-Danlos syndrome (a group of rare genetic syndromes caused by a defect in collagen synthesis).

Men and women can get stretch marks on several areas of their bodies, including the abdominal area, thighs, hips, breasts, upper arms or lower back.

Purported treatment of stretch marks are also currently being marketed, but these treatment are available solely for the purpose of improving the appearance of existing stretch marks. Such treatments for improving the appearance of existing stretch marks include laser treatments, dermabrasion, and topical applications of compositions such as cocoa butter. However, no effective strategy has been described to date for the prevention or cure of stretch marks.

In the methods described herein, stretch marks are treated, e.g., the appearance and fibrosity of the stretch mark or marks is reduced, using pharmacologic intervention using small molecules that modify the behavior of fibroblasts and myofibroblasts in the reticular dermis. Studies have attempted to identify the distribution of extracellular matrix components in skin affected by stretch marks (Watson, et al., Br. J. Dermatol., vol. 138(6):931-937 (1998), hereby incorporated by reference in its entirety), and the contractile forces properties of fibroblasts from stretch marks (Viennet, et al., Arch. Dermatol. Res., vol 297(1): 10-17 (2005), hereby incorporated by reference in its entirety). However, the treatment model described herein is the only method, to date, that targets the formation of striae in a subject by targeting the structures that define the condition. By using small molecule inhibitors to interfere with either the maturation or the action of myofibroblasts, dynamic processes that form the basis for striae appearance and progression are modulated, e.g., prevented or reversed.

Model for Stretch Mark Formation and Progression

In the model for stretch mark formation provided herein, stretch marks are caused by a process of extracellular fiber breakage and tissue micro-tears that, unlike cellulite, occurs primarily in the horizontal plane of the reticular dermis concomitant with skin expansion. As with the proposed model for cellulite, however, a buttressing reaction of myofibroblast-driven contractile ECM deposition is central to the presentation of the condition that manifests as stretch marks. HF, chemically-modified HF, febrifugine, and other compounds that act by diminishing the presence or function of contractile myofibroblasts in the reticular dermis prevent, inhibit progression of, and/or improve the appearance of stretch marks.

Methods for Modulating the Formation and/or Progression of Cellulite and Other Skin Disorders

Suitable modulators of the formation and/or progression of cellulite and other skin disorders include, for example, compositions containing quinazolinones. More particularly, the present invention relates to an anti-cellulite and/or anti-striae composition comprising an amount of quinazolinone derivative as herein defined, effective to inhibit cellulite formation and/or progression, which is therefore useful as a pharmaceutical, as well as a cosmetic, composition.

The invention includes a method for reducing the appearance of or removing cellulite, by administering an effective amount of an anti-cellulite composition comprising a compound of formula I:

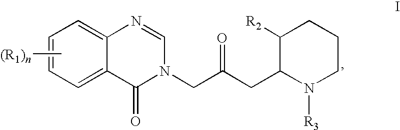

wherein: R₁ is selected from hydrogen, halogen, nitro, benzo, lower alkyl, phenyl and lower alkoxy;

R₂ is selected from hydroxy, acetoxy, and lower alkoxy,

R₃ is selected from hydrogen lower alkoxy-carbonyl and lower alkenoxy-carbonyl, and

n is selected from 1, 2, 3 and 4;

in an amount effective to modulate the formation and/or progression of cellulite in a subject.

The invention includes a method for preventing the appearance of or for reducing the appearance of stretch marks, by administering an effective amount of an anti-striae composition comprising a compound of formula I:

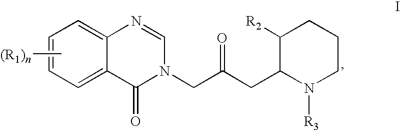

wherein: R₁ is selected from hydrogen, halogen, nitro, benzo, lower alkyl, phenyl and lower alkoxy;

R₂ is selected from hydroxy, acetoxy, and lower alkoxy,

R₃ is selected from hydrogen lower alkoxy-carbonyl and lower alkenoxy-carbonyl, and

n is selected from 1, 2, 3 and 4;

in an amount effective to modulate the formation and/or progression of cellulite in a subject.

The compositions used in the methods of the invention include compounds of formula I and salts, isomers, derivatives, analogs, solvates enantiomers, diasteriomers and/or multimers thereof.

The compositions used in the methods of the invention include acid addition salts.

In certain compounds, n is one. In other compounds, n is two.

In various compounds according to formula I, R₁ is halogen. For example, n is two and both substituents are halogen.

Certain compositions useful in the methods of the invention include an acid addition salt of a compound of formula I. For example, the acid addition salt is a hydrobromide salt.

For example, a compound according to formula I is halofuginone:

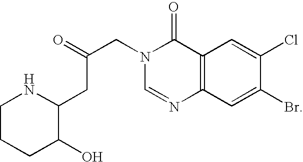

The specific Th17 inhibitors of the invention for use in the treatment of cellulite can be designed, for example, by creating multimers of any of the compounds described above. The invention provides methods of designing suitable specific Th17 inhibitors by linking two or more subunits. In one embodiment, the multimers contain quinazolinone subunits or subunits that are quinazolinone derivatives. The multimer compositions are effective to inhibit or otherwise modulate Th17 development and/or expansion, which is therefore useful as a pharmaceutical composition or as a cosmetic composition, particularly in the treatment of cellulite.

For example, the specific Th17 inhibitor multimers of the invention comprises subunits that comprise a compound of formula I described above or salts, isomers, derivatives, analogs, solvates enantiomers, and/or diasteriomers thereof.

The compositions used in the methods of the invention include compounds of formula I and salts, isomers, derivatives, analogs, solvates enantiomers, diasteriomers and/or multimers thereof.

The compositions used in the methods of the invention include acid addition salts.

In certain compounds, n is one. In other compounds, n is two.

In various compounds according to formula I, R₁ is halogen. For example, n is two and both substituents are halogen.

Certain compositions useful in the methods of the invention include an acid addition salt of a compound of formula I. For example, the acid addition salt is a hydrobromide salt.

For example, a compound according to formula I is halofuginone:

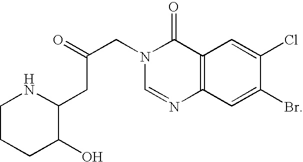

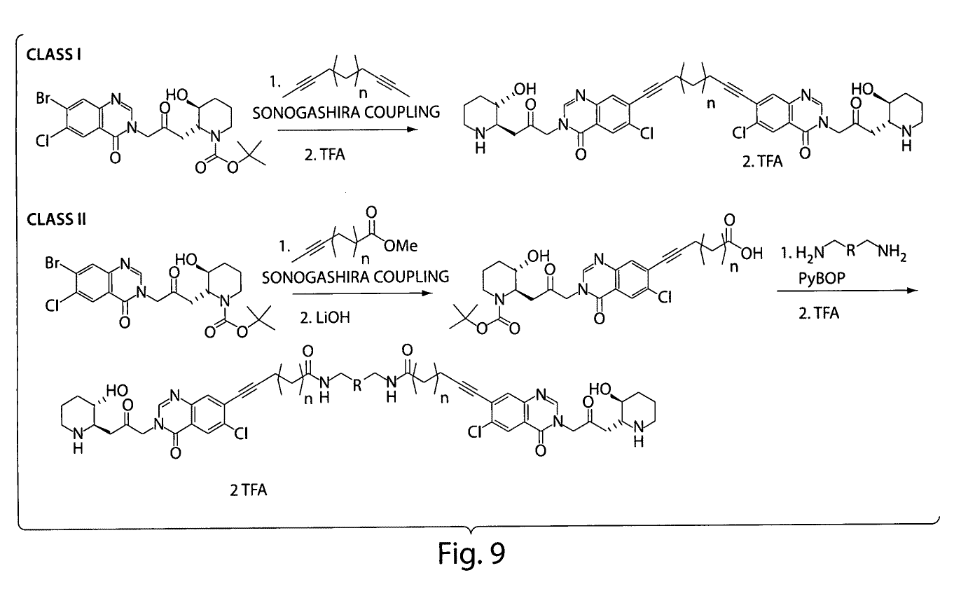

The synthetic strategies for generating HF dimers are outlined in FIG. 9. An exemplary derivative that has been successfully synthesized using this first strategy is shown in FIG. 8.

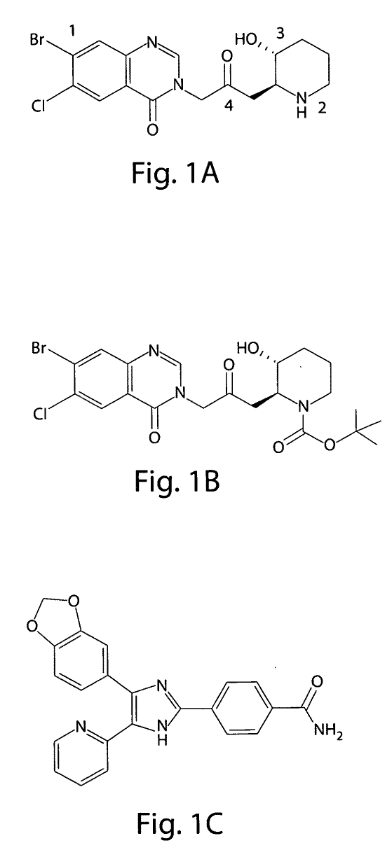

Halofuginone. Halofuginone (HF) (FIG. 1) is a halogenated derivative of febrifugine, a natural product extracted from the roots of the hydrangea Dichroa febrifuga. Dichroa febrifuga is one of the “fifty fundamental herbs” of traditional Chinese medicine, originally used as an anti-malarial remedy (Jiang, et al. Antimicrob Agents Chemother 49, 1169-76 (2005)). Halofuginone, otherwise known as 7-bromo-6-chloro-3-[3-(3-hydroxy-2-piperidinyl)-2-oxopropyl]-4(3H)-quinazolinone, and halofuginone derivatives were described and claimed in U.S. Pat. No. 3,320,124. Febrifugine has been shown to be the active ingredient in Dichroa febrifuga extracts; HF was originally synthesized in search of less toxic anti-malarial derivatives of febrifugine. In addition to its anti-malarial properties, however, HF has striking anti-fibrotic properties in vivo. HF potently reduces dermal extracellular matrix (ECM) deposition with low, in vivo toxicity, which has led to investigation of its utility as a therapeutic for fibrosis, the pathological deposition of ECM (Pines, et al. Biol Blood Marrow Transplant 9, 417-25 (2003)). HF inhibits the transcription of a number of components and modulators of ECM function, including Type I collagen, fibronectin, the matrix metallopeptidases MMP-2 and MMP-9, and the metalloprotease inhibitor TIMP-2 (Li, et al. World J Gastroenterol 11, 3046-50 (2005); Pines, et al. Biol Blood Marrow Transplant 9, 417-25 (2003)). The major cell types responsible for altered ECM deposition, tissue thickening, and contraction during fibrosis are fibroblasts and myofibroblasts. Myofibroblasts mature/differentiate from their precursor fibroblasts in response to cytokine release, often following tissue damage, and mechanical stress, and can be distinguished from fibroblasts by their contractile activity. Excess deposition of ECM, and the differentiation of myofibroblasts that possess contractile activity are central features of fibrosis in a wide range of organs and pathological conditions (Border, et al., New England J. Med., vol. 331: 1286-92 (1994); Branton, et al., Microbes Infect., vol. 1: 1349-65 (1999); Flanders, Int J Exp Pathol vol. 85: 47-64 (2004)). HF, therefore, has been studied extensively as a potential anti-fibrotic therapeutic, and has progressed to phase 2 clinical trials for applications stemming from these properties.

HF acts potently as an inhibitor of fibrosis, at concentrations in the range of 1-200 nM in vitro, and acts specifically, demonstrating low-toxicity in vivo (Pines, et al. Biol Blood Marrow Transplant 9, 417-25 (2003)). In animal models of wound healing and fibrotic disease, HF reduces excess dermal ECM deposition when introduced intra-peritoneally, added to food, or applied locally (Pines, et al. Biol Blood Marrow Transplant 9, 417-25 (2003)). The low toxicity of HF suggests that it does not block any general cellular functions at the doses used for inhibition of fibrosis. HF is currently in Phase II clinical trials as a treatment for scleroderma (Pines, et al. Biol Blood Marrow Transplant 9, 417-25 (2003)), bladder cancer (Elkin, et al., Cancer Res., vol. 59: 4111-18 (1999)), and angiogenesis during Kaposi's Sarcoma, as well as in earlier stages of clinical investigation for a wide range of fibrosis-associated disorders (Nagler, et al. Am J Respir Crit Care Med 154, 1082-86 (1996); Nagler, et al. Arterioscler Thromb Vasc Biol 17, 194-202 (1997); Nagler, et al. Eur J Cancer 40, 1397-403 (2004); Ozcelik, et al. Am J Surg 187, 257-60 (2004)). In spite of the excellent therapeutic promise of HF, very little is known about the molecular mechanisms of HF action. An important recent development has been the demonstration that HF can antagonize the pro-fibrotic activity of the cytokine TGFβ (Xavier, et al., J Biol Chem 279, 15167-76 (2004)) (McGaha, et al. J Invest Dermatol 118, 461-70 (2002)).

While the cellular basis for the anti-fibrotic effect of HF has not been definitively established, published work has focused primarily on the ability of HF to suppress pro-fibrotic gene expression and extracellular matrix secretion by fibroblasts. A single published report has demonstrated a weak, generalized suppression of T-cell proliferation by high doses of HF, but no physiological function has been attributed to this effect of HF. (Lieba et al., J. Leuk. Biol., vol. 80:1-8 (2006)).

The data presented by Lieba et al. demonstrated that anti-CD3 activated human peripheral blood T cells treated with halofuginone display generally reduced levels of cytokine secretion, NF-kB activation and p38 phosphorylation. The oxazolone-induced delayed-type hypersensitivity experiments in mice showed that halofuginone also inhibits this T cell-mediated inflammation (as would be expected from the in vitro experiments). However, all of these effects exhibited by halofuginone seen in the Lieba studies were elicited at high concentrations, with 50% inhibition of these processes only being achieved at 20-40 nM. Moreover, this type of inhibition of T cell function and signaling is most appropriately classified as general inhibition of T cell activation, and as such, halofuginone at these high concentrations behaves similar to a well-characterized T cell activation inhibitor cyclosporine A. Therefore, halofuginone used in this way is a general anti-inflammatory compound, and as such, the use of halofuginone as described by Leiba et al. would prevent global T cell function in the context of an infection, which is a well-known adverse side-effect of other general T cell activation inhibitors, including cyclosporine A.

The data presented herein, in contrast to that described by Leiba et al., clearly demonstrate that while halofuginone at high concentrations (between 20-40 nM) does generally inhibit CD4+ T cell, CD8+ T cell and B220+ B cell activation, HF also very specifically inhibits the development of Th17 cells, i.e., the T helper subset that exclusively expresses high levels of the pro-inflammatory cytokine interleukin (IL)-17. Th17 cells, as a function of their IL-17 secretion play causal roles in the pathogenesis of two important autoimmune diseases in the mouse, experimental autoimmune encephalomyelitis (EAE) and type II collagen-induced arthritis (CIA). EAE and CIA are mouse models of the human autoimmune pathologies multiple sclerosis (MS) and rheumatoid arthritis (RA), respectively.

As shown in the in vitro studies presented herein, halofuginone-mediated, specific inhibition of Th17 cell development takes place at remarkably low concentrations, with 50% inhibition being achieved around 2-3 nM, concentrations which are not associated with any other inhibitory activities previously observed by Leiba et al. (cytokine secretion, NF-kB activation, p38 phosphorylation, delayed-type hypersensitivity). Therefore, halofuginone treatment specifically inhibits the development of Th17-mediated and/or IL-17 related diseases, including autoimmune diseases and infectious diseases, while not leading to profound T cell dysfunction, either in the context of delayed-type hypersensitivity or infection.

Halofuginone modulates Th17 differentiation by stimulating an amino acid starvation response (AAR), which results from the intracellular accumulation of uncharged transfer RNAs (tRNAs). The invention also includes methods for stimulating the AAR using other compounds that cause accumulation of uncharged tRNAs. Examples of such compounds, including, for example, compounds that are structurally unrelated to halofuginone, include histidinol (an inhibitor of histidinyl tRNA charging) (Hansen et al., Reversible inhibition by histidinol of protein synthesis in human cells at the activation of histidine. Biol. Chem. Vol. 247(12):3854-7 (1972)), tryptophanol (an inhibitor of tryptophanyl tRNA charging) (Paley et al., Tryptophanyl-tRNA synthetase in cell lines resistant to tryptophan analogs. Exp. Cell Res. vol. 195(1):66-78 (1991)) and reveromycin (an inhibitor of isoleucyl tRNA synthetase) (Miyamoto et al., Identification of Saccharomyces cerevisiae isoleucyl-tRNA synthetase as a target of the G1-specific inhibitor Reveromycin A., J Biol Chem. vol.; 277(32):28810-4. Epub 2002).

As used herein, the term “alkyl” includes saturated aliphatic groups, including straight-chain alkyl groups (e.g., methyl, ethyl, propyl, butyl, pentyl, hexyl) and branched-chain alkyl groups (e.g., isopropyl, tert-butyl, isobutyl. In certain embodiments, a straight chain or branched chain alkyl has six or fewer carbon atoms in its backbone (e.g., C₁-C₆ for straight chain, C₃-C₆ for branched chain), and in other embodiments four or fewer carbon atoms. Lower alkyl groups include from 1-6 carbon atoms, thus the term “lower alkyl” includes alkyl groups containing 1, 2, 3, 4, 5, or 6 carbon atoms.

The term “alkoxy” or “alkoxyl” includes substituted and unsubstituted alkyl groups covalently linked to an oxygen atom. Examples of alkoxy groups (or alkoxyl radicals) include methoxy, ethoxy, isopropyloxy, propoxy, butoxy, and pentoxy groups. Examples of substituted alkoxy groups include halogenated alkoxy groups. The alkoxy groups can be substituted with groups such as alkenyl, alkynyl, halogen, hydroxyl, carboxylate, alkoxyl, cyano, amino (including —NH₂, alkylamino, dialkylamino, arylamino, diarylamino, and alkylarylamino), nitro, trifluoromethyl, cyano, azido, heterocyclyl, or an aromatic or heteroaromatic moiety. Examples of halogen substituted alkoxy groups include, but are not limited to, fluoromethoxy, difluoromethoxy, trifluoromethoxy, chloromethoxy, dichloromethoxy, and trichloromethoxy. Lower alkoxy groups include from 1-6 carbon atoms, thus the term “lower alkoxy” includes alkyl groups containing 1, 2, 3, 4, 5, or 6 carbon atoms.

The term “hydroxy” or “hydroxyl” includes groups with an —OH or —O⁻.

The term “halogen” includes fluorine, bromine, chlorine, iodine, etc. The term “perhalogenated” generally refers to a moiety wherein all hydrogens are replaced by halogen atoms.

In the present specification, the structural formula of the compound represents a certain isomer for convenience in some cases, but the present invention includes all isomers such as geometrical isomer, optical isomer based on an asymmetrical carbon, stereoisomer, tautomer and the like which occur structurally and an isomer mixture and is not limited to the description of the formula for convenience, and may be any one of isomer or a mixture. Therefore, an asymmetrical carbon atom may be present in the molecule and an optically active compound and a racemic compound may be present in the present compound, but the present invention is not limited to them and includes any one. In addition, a crystal polymorphism may be present but is not limiting, but any crystal form may be single or a crystal form mixture, or an anhydride or hydrate. Further, so-called metabolite which is produced by degradation of the present compound in vivo is included in the scope of the present invention.

It will be noted that the structure of some of the compounds of the invention include asymmetric (chiral) carbon atoms. It is to be understood accordingly that the isomers arising from such asymmetry are included within the scope of the invention, unless indicated otherwise. Such isomers can be obtained in substantially pure form by classical separation techniques and by stereochemically controlled synthesis. The compounds of this invention may exist in stereoisomeric form, therefore can be produced as individual stereoisomers or as mixtures.

“Isomerism” means compounds that have identical molecular formulae but that differ in the nature or the sequence of bonding of their atoms or in the arrangement of their atoms in space. Isomers that differ in the arrangement of their atoms in space are termed “stereoisomers”. Stereoisomers that are not mirror images of one another are termed “diastereoisomers”, and stereoisomers that are non-superimposable mirror images are termed “enantiomers”, or sometimes optical isomers. A carbon atom bonded to four nonidentical substituents is termed a “chiral center”.

“Chiral isomer” means a compound with at least one chiral center. It has two enantiomeric forms of opposite chirality and may exist either as an individual enantiomer or as a mixture of enantiomers. A mixture containing equal amounts of individual enantiomeric forms of opposite chirality is termed a “racemic mixture”. A compound that has more than one chiral center has 2ⁿ⁻¹ enantiomeric pairs, where n is the number of chiral centers. Compounds with more than one chiral center may exist as either an individual diastereomer or as a mixture of diastereomers, termed a “diastereomeric mixture”. When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al, Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J., Chem. Educ. 1964, 41, 116).

“Geometric Isomers” means the diastereomers that owe their existence to hindered rotation about double bonds. These configurations are differentiated in their names by the prefixes cis and trans, or Z and E, which indicate that the groups are on the same or opposite side of the double bond in the molecule according to the Cahn-Ingold-Prelog rules.

Further, the structures and other compounds discussed in this application include all atropic isomers thereof. “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

The terms “crystal polymorphs” or “polymorphs” or “crystal forms” means crystal structures in which a compound (or salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds can be prepared by crystallization under different conditions.

Additionally, the compounds of the present invention, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

“Solvates” means solvent addition forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate.

“Tautomers” refers to compounds whose structures differ markedly in arrangement of atoms, but which exist in easy and rapid equilibrium. It is to be understood that compounds of Formula I may be depicted as different tautomers. It should also be understood that when compounds have tautomeric forms, all tautomeric forms are intended to be within the scope of the invention, and the naming of the compounds does not exclude any tautomer form.

Some compounds of the present invention can exist in a tautomeric form which are also intended to be encompassed within the scope of the present invention.

The compounds, salts and prodrugs of the present invention can exist in several tautomeric forms, including the enol and imine form, and the keto and enamine form and geometric isomers and mixtures thereof. All such tautomeric forms are included within the scope of the present invention. Tautomers exist as mixtures of a tautomeric set in solution. In solid form, usually one tautomer predominates. Even though one tautomer may be described, the present invention includes all tautomers of the present compounds

A tautomer is one of two or more structural isomers that exist in equilibrium and are readily converted from one isomeric form to another. This reaction results in the formal migration of a hydrogen atom accompanied by a switch of adjacent conjugated double bonds. In solutions where tautomerization is possible, a chemical equilibrium of the tautomers will be reached. The exact ratio of the tautomers depends on several factors, including temperature, solvent, and pH. The concept of tautomers that are interconvertable by tautomerizations is called tautomerism.

Of the various types of tautomerism that are possible, two are commonly observed. In keto-enol tautomerism a simultaneous shift of electrons and a hydrogen atom occurs. Ring-chain tautomerism, is exhibited by glucose. It arises as a result of the aldehyde group (—CHO) in a sugar chain molecule reacting with one of the hydroxy groups (—OH) in the same molecule to give it a cyclic (ring-shaped) form.

Tautomerizations are catalyzed by: Base: 1. deprotonation; 2. formation of a delocalized anion (e.g. an enolate); 3. protonation at a different position of the anion; Acid: 1. protonation; 2. formation of a delocalized cation; 3. deprotonation at a different position adjacent to the cation.

Common tautomeric pairs are: ketone-enol, amide-nitrile, lactam-lactim, amide-imidic acid tautomerism in heterocyclic rings (e.g. in the nucleobases guanine, thymine, and cytosine), amine-enamine and enamine-enamine. Examples include:

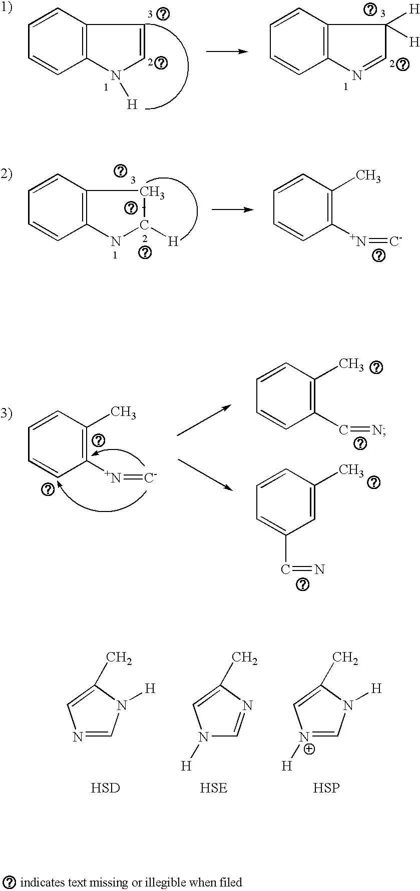

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As defined herein, the term “derivative”, refers to compounds that have a common core structure, and are substituted with various groups as described herein. For example, all of the compounds represented by formula I are indole derivatives, and have formula I as a common core.

The term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include acyl sulfonamides, tetrazoles, sulfonates, and phosphonates. See, e.g., Patani and LaVoie, Chem. Rev. 96, 3147-3176 (1996).

A “pharmaceutical composition” is a formulation containing the disclosed compounds in a form suitable for administration to a subject.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

The compounds of the invention are capable of further forming salts. All of these forms are also contemplated within the scope of the claimed invention. For example, the salt can be an acid addition salt. One example of an acid addition salt is a hydrochloride salt. Another example is a hydrobromide salt.

“Pharmaceutically acceptable salt” of a compound means a salt that is pharmaceutically acceptable and that possesses the desired pharmacological activity of the parent compound.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The invention also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It should be understood that all references to pharmaceutically acceptable salts include solvent addition forms (solvates) or crystal forms (polymorphs) as defined herein, of the same salt.

The pharmaceutically acceptable salts of the present invention can be synthesized from a parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 18th ed. (Mack Publishing Company, 1990). For example, salts can include, but are not limited to, the hydrochloride and acetate salts of the aliphatic amine-containing, hydroxyl amine-containing, and imine-containing compounds of the present invention.

The compounds of the present invention can also be prepared as prodrugs, for example pharmaceutically acceptable prodrugs. The terms “pro-drug” and “prodrug” are used interchangeably herein and refer to any compound which releases an active parent drug in vivo. Since prodrugs are known to enhance numerous desirable qualities of pharmaceuticals (e.g., solubility, bioavailability, manufacturing, etc.) the compounds of the present invention can be delivered in prodrug form. Thus, the present invention is intended to cover prodrugs of the presently claimed compounds, methods of delivering the same and compositions containing the same. “Prodrugs” are intended to include any covalently bonded carriers that release an active parent drug of the present invention in vivo when such prodrug is administered to a subject. Prodrugs the present invention are prepared by modifying functional groups present in the compound in such a way that the modifications are cleaved, either in routine manipulation or in vivo, to the parent compound. Prodrugs include compounds of the present invention wherein a hydroxy, amino, sulfhydryl, carboxy, or carbonyl group is bonded to any group that, may be cleaved in vivo to form a free hydroxyl, free amino, free sulfhydryl, free carboxy or free carbonyl group, respectively.

Examples of prodrugs include, but are not limited to, esters (e.g., acetate, dialkylaminoacetates, formates, phosphates, sulfates, and benzoate derivatives) and carbamates (e.g., N,N-dimethylaminocarbonyl) of hydroxy functional groups, esters groups (e.g. ethyl esters, morpholinoethanol esters) of carboxyl functional groups, N-acyl derivatives (e.g. N-acetyl) N-Mannich bases, Schiff bases and enaminones of amino functional groups, oximes, acetals, ketals and enol esters of ketone and aldehyde functional groups in compounds of formula I, and the like, See Bundegaard, H. “Design of Prodrugs” p 1-92, Elesevier, New York—Oxford (1985).

“Protecting group” refers to a grouping of atoms that when attached to a reactive group in a molecule masks, reduces or prevents that reactivity. Examples of protecting groups can be found in Green and Wuts, Protective Groups in Organic Chemistry, (Wiley, 2^{nd} ed. 1991); Harrison and Harrison et al., Compendium of Synthetic Organic Methods, Vols. 1-8 (John Wiley and Sons, 1971-1996); and Kocienski, Protecting Groups, (Verlag, 3^{rd} ed. 2003).

For example, representative hydroxy protecting groups include those where the hydroxy group is either acylated or alkylated such as benzyl, and trityl ethers as well as alkyl ethers, tetrahydropyranyl ethers, trialkylsilyl ethers and allyl ethers.

Stable compound” and “stable structure” are meant to indicate a compound that is sufficiently robust to survive isolation to a useful degree of purity from a reaction mixture, and formulation into an efficacious therapeutic agent.

Screening Methods

In addition to the derivatives of HF described above, the invention provides methods of screening to identify compounds that possess similar biological activity to the HF class of specific Th17 inhibitors, e.g., the ability to modulate the formation and/or progression of a cellulite and/or other skin disorders in a subject. Thus, these screening methods are used to identify compounds that are functionally similar to the HF class of compounds, but are not necessarily structurally similar to the HF class of compounds.

For example, the invention provides a method (also referred to herein as a “screening assay”) for identifying modulators, i.e., candidate or test compounds or agents (e.g., peptides, peptidomimetics, small molecules or other drugs) that modulate the formation and/or progression of cellulite and other skin disorders. Additional modulators of the formation and/or progression of cellulite and other skin disorders can be identified using any of a variety of screening methods known in the art. The invention further encompasses novel agents identified by the screening assays described herein.

The invention provides assays for screening candidate or test compounds which bind to or modulate the formation and/or progression of cellulite and other skin disorders in a subject. The test compounds of the invention can be obtained using any of the numerous approaches in combinatorial library methods known in the art, including: biological libraries; spatially addressable parallel solid phase or solution phase libraries; synthetic library methods requiring deconvolution; the “one bead one compound” library method; and synthetic library methods using affinity chromatography selection. The biological library approach is limited to peptide libraries, while the other four approaches are applicable to peptide, non peptide oligomer or small molecule libraries of compounds. See, e.g., Lam, 1997. Anticancer Drug Design 12: 145.

A “small molecule” as used herein, is meant to refer to a composition that has a molecular weight in the range of less than about 5 kD to 50 daltons, for example less than about 4 kD, less than about 3.5 kD, less than about 3 kD, less than about 2.5 kD, less than about 2 kD, less than about 1.5 kD, less than about 1 kD, less than 750 daltons, less than 500 daltons, less than about 450 daltons, less than about 400 daltons, less than about 350 daltons, less than 300 daltons, less than 250 daltons, less than about 200 daltons, less than about 15.0 daltons, less than about 100 daltons. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412 421), or on beads (Lam, 1991. Nature 354: 82 84), on chips (Fodor, 1993. Nature 364: 555 556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865 1869) or on phage (Scott and Smith, 1990. Science 249: 386 390; Devlin, 1990. Science 249: 404 406; Cwirla, et al., 1990. Proc. Natl. Acad. Sci. U.S.A. 87: 6378 6382; Felici, 1991. J. Mol. Biol. 222: 301 310; Ladner, U.S. Pat. No. 5,233,409).

An assay is a cell based assay in which a cell which expresses a membrane bound form of an HF binding protein or a biologically active portion thereof, on the cell surface is contacted with a test compound and the ability of the test compound to bind to the HF binding protein is determined. Determining the ability of the test compound to bind to the HF binding protein can be accomplished, for example, by coupling the test compound with a radioisotope or enzymatic label such that binding of the test compound to the HF binding protein or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane bound form of an HF binding protein, or a biologically active portion thereof, on the cell surface with a HF, or a derivative thereof, to form an assay mixture, contacting the assay mixture with a test compound, and determining the ability of the test compound to compete with HF and to interact with the HF binding protein, wherein determining the ability of the test compound to interact with the HF binding protein comprises determining the ability of the test compound to preferentially bind to the HF binding protein or a biologically active portion thereof as compared to HF, or a derivative thereof.

Inhibitors of cellulite formation are also identified by observing the effect of the test compound on fibroblast behavior. In this assay, a control population of primary dermal fibroblasts are cultured in a matrix of Type I collagen, mimicking the interstitial matrix of the dermis and hypodermis, such that the fibroblasts attach to the substratum and spread, exerting contractile force on the surrounding interstitial matrix. In the test population, primary dermal fibroblasts are incubated on a matrix of Type I collagen in the presence of a test compound for a predetermined length of time, e.g., 24 hrs. The ability of the dermal fibroblasts to attach to the substratum and spread is then observed. Failure of the dermal fibroblasts to attach and spread indicates that the test compound is a putative inhibitor of cellulite formation.

Yet another approach for identifying inhibitors of cellulite formation is an assay in which a test compound is administered to a tissue test area in a subject exhibiting cellulite. The contractile force exerted by vertical collagen fibers tethered between fascia and dermo-hypodermal interface within the tissue test site is measured and compared to the contractile force exerted by vertical collagen fibers in a second tissue site exhibiting cellulite in the subject. A decrease in the contractile force in the tissue test area indicates that the test compound is an inhibitor of cellulite formation.

Methods of Treating Cellulite

In addition to the currently investigated use of HF as a therapeutic for fibrosis-associated diseases, HF, febrifugine, and related compounds are effective agents in the prevention and treatment of the skin condition cellulite. It has been observed that HF prevents the spreading and contractile activity of dermal fibroblasts, a cellular function that is believed to mediate tissue changes during cellulite formation and progression. HF, febrifugine, and related compounds are used herein for the treatment of cellulite because 1) the action of HF and small molecules that are functionally related to HF, e.g., molecules identified using the screening methods described herein, are antagonistic to the appearance of cellulite based on the model for cellulite formation and progression described above, 2) these small molecules are readily delivered to the cellulite-affected areas through a topical cream formulation and can be used in combination with treatments that improve the general appearance of the skin, i.e., laser and mechanical manipulation 3) these compounds can be used effectively and with low-toxicity for the treatment of cellulite whether they are delivered topically, orally, or through subcutaneous injection based on the human trial data for other applications of HF. The preferred method for the application of HF or related compounds is a topical cream containing either HF or its plant-derived precursor febrifugine. Oral therapy or subcutaneous injection would be alternative methods for the delivery of the cellulite antagonists.

Compositions formulated for treatment of cellulite are targeted for maximum penetrance in the hypodermis of the subject. For example, in topical compositions designed to treat the formation and/or progression of cellulite, the penetrance of each topical formulation is compared and tested for tissue bioavailability, e.g., by using pig skin, which is an excellent model for human skin. The penetrance of each formulation for the treatment and/or reduction of cellulite is tested, for example, using blood level analysis, animal models or a combination of both. For example, a formulation is applied to pig's skin, and after a suitable period post-application, a sample is removed from the test area, e.g., using a punch biopsy. The sample is then analyzed to determine to detect the presence or absence of the formulation, and of the active ingredient in particular, within the skin sample. Alternatively or in addition to this biopsy procedure, blood samples are analyzed to determine whether the formulation, and in particular, the active ingredient of the formulation, e.g., HF or its plant-derived precursor febrifugine, is present in the blood, serum or other blood component of the subject. In human patients, penetrance of the formulation is determined, for example, using blood level analysis, biopsy analysis, or a combination of both.

Cellulite inhibitors include compounds that act to inhibit expansion of Th17 cells, or that inhibit cytokine-driven pathways “downstream” of this T-cell response. One means of boosting efficacy of a cellulite treatment, while avoiding and/or minimizing toxicity issues, is to use a combination of compounds that act in the same, or signal reinforcing, pathway(s). Two classes of small molecule currently in common use for other indications that might synergize with HF as topical anti-cellulite agents are:

1) Statins. In addition to their well established activity as cholesterol-lowering agents via inhibition of HMG coA-reductase, statins have several anti-fibrotic and anti-inflammatory effects. For example, pravastatin has been shown to inhibit inflammation-associated fibrosis, e.g. chronic cyclosporine A nephropathy in rats through the inhibition of a TGFbeta-inducible gene, betaIG-H3. (Li et al., Am J Nephrol. 2005 November-December; 25(6):611-20). Statins have also been shown to have beneficial effects in the treatment of central nervous system autoimmune diseases, such that states are now being tested in patients with multiple sclerosis in clinical trials. (Weber et al., J. Neuroimmunol., vol. 178: 140-148 (2006)).

Suitable statins for use in co-therapy methods of the invention include, for example, atorvastatin, lovastatin, compactin, mevinolin, pravastatin, fluvastatin, mevastatin, visastatin/rosuvastatin, velostatin, cerivastatin, simvastatin, synvinolin, rivastatin (sodium 7-(4-fluorophenyl)-2,6-diisoprop-yl-5-methoxymethylpyridin-3-yl)-3,5-dihydroxy-6-heptanoate), itavastatin/pitavastatin, pharmaceutically acceptable salts and esters thereof, and combinations thereof. In some embodiments, the combination of a compound of the invention and a statin produces synergistic effect on the modulation of Th17 development, and particularly, in the modulation of the formation and/or progression of cellulite. In these synergistic combinations, the statin itself (or combination of statins) need not be able to inhibit, treat or otherwise the delay the progression of cellulite in a subject, provided that, when the statin (or combination of statins) is co-administered with a compound of the invention, the resulting co-therapy compositions is able to inhibit, treat or otherwise the delay the progression of cellulite.

2) Retinoic acid. Studies have demonstrated that retinoic acid inhibits RORgammat (RORγt), a key regulator of Th17 development. (Mucida et al., Science, epublication Jun. 18, 2007). In some embodiments, the combination of a compound of the invention and retinoic acid produces synergistic effect on the modulation of Th17 development, and particularly, in the modulation of the formation and/or progression of cellulite. In these synergistic combinations, retinoic acid itself need not be able to inhibit, treat or otherwise the delay the progression of cellulite in a subject, provided that, when retinoic acid is co-administered with a compound of the invention, the resulting co-therapy compositions is able to inhibit, treat or otherwise the delay the progression of cellulite.

Methods are also provided for the identification of the cellular targets of HF that mediate its action, e.g., methods for the identification of additional classes of small molecules that affect the same cellular regulatory pathways as HF. Small molecules identified by this approach and delivered topically, orally, or by subcutaneous injection would constitute an additional class of treatments for cellulite. Topical creams that prevent or reverse the characteristic dimpling seen with this condition easily could be used in combination with treatments such as laser. The impact of laser treatments would be to act by increasing the amount of collagen in the dermis, helping to disguise the effects of the discontinuities at the dermo-hypodermal connective tissue border, a minimal protrusion of fat, by creating plumper skin. The ubiquitous gender-linked discontinuities of this connective tissue border likely will not be impacted by HF treatments, but these structural features are not, by themselves, responsible cellulite presentation. Laser treatments likely will be minimally effective alone, requiring frequent treatment for minor short-term improvement at greater expense. A combined treatment approach using laser treatment along with a mechanistically-based topical cream that directly addresses/reverses skin dimpling could improve the delivery and penetration of the active compound into the deep hypodermis, and provide a further general enhancement of the skin's appearance.

Without wishing to be bound by theory, laser treatment is thought to augment the penetration of a topically-applied ECM-remodeling compound of the invention. Further, topical creams or gels containing a compound of the invention capable of remodeling the ECM can be used in conjunction with laser treatments, mechanical manipulation, or both.

One tissue treatment involves the application of laser radiation to an area targeted for, cellulite reduction, in a predetermined wavelength, or combination of wavelengths, intervals, periods, and patterns. The application of infrared radiation can optionally follow. Optionally, subsequent to the application of infrared radiation, the tissue can be massaged using a combination of massage techniques.

The duration, power, and wavelengths of the application of the laser depends primarily on a formulation of different factors, such as calf circumference, thigh circumference, abdominal circumference, hip circumference, patient weight, and patient height.

For example, a patient having a thigh circumference of 38 cm, calf circumference of 31 cm, abdominal circumference of 76 cm, hip circumference of 82 cm, weight of 62 kg, ideal body weight of 50 kg according to Standard Metropolitan Life Insurance Company Tables, and height of 5 feet 3 inches, would be exposed to the rastered radiation for 6.4 minutes per area. The time duration for exposure of each area on the skin surface is calculated based on the following expression: (Hip Circumference in cm)/(Abdominal Circumference in cm)×(Weight in kgs)/(Ideal Body Weight in kgs)×(Calf Circumference in cm+Thigh Circumference in cm)×Reduction Factor=Time in Minutes. Ideal Body Weight (“IBW”) is as defined by Standard Metropolitan Life Insurance Company tables for IBW calculated from the patient's height. The Reduction Factor is 0.07 with a minimum exposure of 5.5 minutes per area. The maximum exposure per area for a majority of the patients is about 12 minutes. However, this number may vary depending on the above variables. Again, the power of the laser 12 in the described embodiment is fixed. However, a laser having variable power settings can be employed, and the above relationship of variables adjusted for different power settings.

The typical exposure time per area being exposed is between about five and twelve minutes for an individual female adult of average build. For example, the thigh area can be treated in three sections, each area encompassing 120 degrees of the thigh with approximately 20% overlap. Thus, for each thigh, the laser treatment time is approximately three times the duration of exposure for each area, or eighteen to thirty minutes per limb. The duration of exposure varies depending on the actual values of the above-mentioned variables. The laser is delivered as a continuous wave beam rastered across the skin surface. The entire skin area to be treated may be covered by a single scan at a rate of about 1 Hz.

Examples of laser systems useful in combination with the compounds of the invention include VELASMOOTH™ (Syneron Inc, Richmond Hill, Ontario, CA), which combines near infrared light at a wavelength of 700 nm, continuous wave radiofrequency and mechanical suction, and TRI-ACTIVE™ Laserdermology (Cynosure Inc., Chelmsford Mass.), which combines six near-infrared diode lasers at a wavelength of 810 nm, localized cooling, and mechanical massage.

Methods of Treating or Preventing Stretch Marks

Topical delivery of HF, modified HF, febrifugine, or other functionally-related compounds in a vehicle with limited dermal penetrance are used to prevent and improve the appearance of the other skin disorders such as, e.g., stretch marks. In contrast to the condition cellulite, therapeutics for stretch marks need not gain access to tissues beneath the reticular dermis. In particular, compositions formulated for treatment of stretch marks are targeted for maximum penetrance in the dermis of the subject. Since the appearance of stretch marks occurs frequently and predictably during pregnancy, the design of any effective preventative, or therapeutic should take into account this physiologic condition. For protection of the fetus, limited tissue penetrance of a topical cosmetic agent is desirable for treatments intended for use during pregnancy, and can be obtained by modification of either the active compound or its application vehicle.

The compositions provided herein include, for example, a limited-penetrance derivative of HF for the treatment of stretch marks. Alternatively or in addition, topical creams that contain febrifugine, HF, and HF derivatives are designed to have limited penetrance, e.g., the compound is preferentially retained at the dermis. The penetrance of each topical formulation is compared and tested for tissue bioavailability, for example, in pig skin, which is an excellent model for human skin. The penetrance of each topical formulation is compared and tested to ensure that the formulation is not present in the bloodstream of the subject or animal model. The penetrance of each formulation for the treatment of stretch marks is tested, for example, using blood level analysis, animal models or a combination of both. For example, a formulation designed to treat or prevent stretch marks is applied to pig's skin, and after a suitable period post-application, a sample is removed from the test area. The sample is then analyzed to determine to detect the presence or absence of the formulation, and of the active ingredient in particular, within the skin sample. Alternatively or in addition to this biopsy procedure, blood samples are analyzed to determine whether the formulation, and in particular, the active ingredient of the formulation, e.g., HF or its plant-derived precursor febrifugine, is present in the blood, serum or other blood component of the subject. In human patients, penetrance of the formulation for treating or preventing stretch marks is determined, for example, using blood level analysis.

The methods and compositions for preventing the appearance of or reducing the appearance of stretch marks can be used in conjunction with a second treatment regime. For example, the compositions of provided herein can be used in conjunction with dermabrasion, chemical peel, and/or laser treatments. The methods and compositions for preventing the appearance of or reducing the appearance of stretch marks can be used in conjunction with other topical agents such as, e.g., cocoa butter, compositions comprising vitamin A and/or vitamin E, and other commercially available over the counter products.

Determination of the Biological Effect of an Anti-Cellulite Therapeutic

In various embodiments of the invention, suitable in vitro or in vivo studies are performed to determine the effect of a specific anti-cellulite therapeutic and whether its administration is indicated for treatment of the affected tissue exhibiting cellulite. For example, the biological effect of an anti-cellulite therapeutic, such as HF, is monitored by measuring the contractile force exerted by vertical collagen fibers tethered between fascia and dermo-hypodermal interface in a tissue site that has been treated with the therapeutic. The biological effect of a therapeutic is also measured by observing the tissue structure after treatment using, for example, magnetic resonance imaging (MRI) of the treated area. Alternatively, or in addition, the biological effect is monitored by physical observation of the appearance of the treated tissue site in a subject, e.g., observing the extent of skin dimpling within the treated area and/or observing the depth of each dimple in the treated area. Typically, the observation is performed by a plastic surgeon.

An assay is provided to test the effects of HF and other selective Th17 inhibitors on the behavior of cells likely to provide the basis for the structural features of cellulite—dermal fibroblasts and myofibroblasts. When human primary dermal fibroblasts are cultured in a matrix of Type I collagen, mimicking the interstitial matrix of the dermis and hypodermis, they attach to the substratum and spread, exerting contractile force on the surrounding interstitial matrix. In this assay, primary dermal fibroblasts are incubated on a matrix of Type I collagen for 24 hrs in the presence of HF or other selective Th17 inhibitor. The ability of the dermal fibroblasts to attach and spread when cultured in the presence of the Th17 inhibitor is detected. A reduction in the ability of the dermal fibroblasts to attach and spread indicates that the selective Th17 inhibitor is effective in treating or preventing the formation and/or progression of cellulite.

Pharmaceutical Compositions and Formulations

The modulators of Th17 cell development and/or expansion, and precursors, prodrugs, derivatives, fragments, analogs and homologs thereof, (also referred to herein as “active ingredients”) can be incorporated into pharmaceutical compositions suitable for administration. In addition, the modulators of cellulite formation and/or progression and modulator of stretch mark formation and/or progression, and precursors, prodrugs, derivatives, fragments, analogs and homologs thereof, (also referred to herein as “active ingredients”) can be incorporated into pharmaceutical compositions suitable for administration. Such compositions typically comprise the modulator and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences. Preferred examples of such carriers or diluents include, but are not limited to, water, saline, finger's solutions, dextrose solution, and 5% human serum albumin. Liposomes and non aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions. Pharmaceutical compositions containing one or more active ingredients, e.g., one or more modulators of cellulite formation and/or progression, and precursors, prodrugs, derivatives, fragments, analogs and homologs thereof, are formulated as prescription formulations, or alternatively as over-the-counter formulations.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Pharmaceutical compositions formulated for topical administration for the treatment of cellulite contain the active ingredient(s) in an amount that sufficient to modulate Th17 cell development. Preferably, the pharmaceutical compositions for topical administration are formulated such that the specific inhibition of Th17 observed in vivo in the subject is comparable to the specific inhibition of Th17 differentiation that is observed in vitro when a population of Th17 cells is contacted with a concentration window of 2-30 nM of exogenously added HF.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Formulations suitable for topical administration include liquid or semi-liquid preparations such as liniments, lotions, gels, applicants, oil-in-water or water-in-oil emulsions such as creams, ointments or pastes; or solutions or suspensions such as drops. Formulations for topical administration to the skin surface can be prepared by dispersing the drug with a dermatologically acceptable carrier such as a lotion, cream, ointment or soap. Useful are carriers capable of forming a film or layer over the skin to localize application and inhibit removal. For topical administration to internal tissue surfaces, the agent can be dispersed in a liquid tissue adhesive or other substance known to enhance adsorption to a tissue surface. For example, hydroxypropylcellulose or fibrinogen/thrombin solutions can be used to advantage. Alternatively, tissue-coating solutions, such as pectin-containing formulations can be used.

Additionally, the carrier for a topical formulation can be in the form of a hydroalcoholic system (e.g. quids and gels), an anhydrous oil or silicone based system, or an emulsion system, including, but not limited to, oil-in-water, water-in-oil, water-in-oil-in-water, and oil-in-water-in-silicone emulsions. The emulsions can cover a broad range of consistencies including thin lotions (which can also be suitable for spray or aerosol delivery), creamy lotions, light creams, heavy creams, and the like. The emulsions can also include microemulsion systems. Other suitable topical carriers include anhydrous solids and semisolids (such as gels and sticks); and aqueous based mousse systems. Nonlimiting examples of the topical carrier systems useful in the present invention are described in the following four references: “Sun Products Formulary”, Cosmetics & Toiletries, vol. 105, pp. 122-139 (December 1990); “Sun Products Formulary”, Cosmetics & Toiletries, vol. 102, pp. 117-136 (March 1987); U.S. Pat. No. 4,960,764; and U.S. Pat. No. 4,254,105.

The following components are useful for topical compositions:

### Humectants, Moisturizers, and Skin Conditioners

Particularly for topical compositions, optional component of the compositions useful in the instant invention is at least one humectant/moisturizer/skin conditioner. A variety of these materials can be employed and each can be present at a level of from about 0.1% to about 20%, alternatively from about 1% to about 10% and yet alternatively from about 2% to about 5%. These materials include urea; guanidine; glycolic acid and glycolate salts (e.g. ammonium and quaternary alkyl ammonium); lactic acid and lactate salts (e.g. ammonium and quaternary alkyl ammonium); aloe vera in any of its variety of forms (e.g., aloe vera gel); polyhydroxy alcohols such as sorbitol, glycerol, hexanetriol, propylene glycol, hexylene glycol and the like; polyethylene glycol; sugars and starches; sugar and starch derivatives (e.g., alkoxylated glucose); hyaluronic acid; lactamide monoethanolamine; acetamide monoethanolamine; and mixtures thereof.

In certain embodiments for topical compositions, humectants/moisturizers/skin conditioners useful herein are the C₃-C₆ diols and triols, and also aloe vera gel. Especially preferred is the triol, glycerol, and also aloe vera gel.

### Surfactants

The compositions useful in the methods of the present invention, particularly the topical compositions, can optionally comprise one or more surfactants. The surfactants can be present at a level from about 0.1% to about 10%, alternatively from about 0.2% to about 5%, and yet alternatively from about 0.2% to about 2.5%. Suitable surfactants include, but are not limited to, nonionic surfactants such as polyalkylene glycol ethers of fatty alcohols, and anionic surfactants such as taurates and alkyl sulfates. Nonlimiting examples of these surfactants include isoceteth-20, sodium methyl cocoyl taurate, sodium methyl oleoyl taurate, and sodium lauryl sulfate. See U.S. Pat. No. 4,800,197. Examples of a broad variety of additional surfactants useful herein are described in McCutcheon's, Detergents and Emulsifiers, North American Edition (1986), published by Allured Publishing Corporation.

### Emollients

The compositions useful in the methods of the present invention, particularly topical compositions, can also optionally comprise at least one emollient. Examples of suitable emollients include, but are not limited to, volatile and non-volatile silicone oils, highly branched hydrocarbons, and non-polar carboxylic acid and alcohol esters, and mixtures thereof. Emollients useful in the instant invention are further described in U.S. Pat. No. 4,919,934.

The emollients can typically comprise in total from about 1% to about 50%, preferably from about 1% to about 25%, and more preferably from about 1% to about 10% by weight of the compositions useful in the present invention.

### Sunscreens

The compositions useful in the methods of the present invention for topical administration can also optionally comprise at least one sun screening agent. A wide variety of one or more sun screening agents are suitable for use in the present invention and are described in U.S. Pat. No. 5,087,445; U.S. Pat. No. 5,073,372; U.S. Pat. No. 5,073,371; and Segarin, et al., at Chapter VIII, pages 189 et seq., of Cosmetics Science and Technology.

Certain useful in the compositions of the instant invention ethylhexyl p-methoxycinnamate, octocrylene, octyl salicylate, oxybenzone, or mixtures thereof. Other useful sunscreens include the solid physical sunblocks such as titanium dioxide (micronized titanium dioxide, 0.03 microns), zinc oxide, silica, iron oxide and the like. Without being limited by theory, it is believed that these inorganic materials provide a sun screening benefit through reflecting, scattering, and absorbing harmful UV, visible, and infrared radiation.

Still other useful sunscreens are those disclosed in U.S. Pat. No. 4,937,370; and U.S. Pat. No. 4,999,186. The sun screening agents disclosed therein have, in a single molecule, two distinct chromophore moieties which exhibit different ultra-violet radiation absorption spectra. One of the chromophore moieties absorbs predominantly in the UVB radiation range and the other absorbs strongly in the UVA radiation range. These sun screening agents provide higher efficacy, broader UV absorption, lower skin penetration and longer lasting efficacy relative to conventional sunscreens.

Generally, the sunscreens can comprise from about 0.5% to about 20% of the compositions useful herein. Exact amounts will vary depending upon the sunscreen chosen and the desired Sun Protection Factor (SPF). SPF is a commonly used measure of photoprotection of a sunscreen against erythema. See Federal Register, Vol. 43, No. 166, pp. 38206-38269, Aug. 25, 1978.

The topical compositions useful for the methods of the instant invention can also be delivered from a variety of delivery devices. For example, the compositions useful herein can be incorporated into a medicated cleansing pad. Preferably these pads comprise from about 50% to about 75% by weight of one or more layers of nonwoven fabric material and from about 20% to about 75% by weight (on dry solids basis) of a water soluble polymeric resin. These pads are described in detail in U.S. Pat. No. 4,891,228 and U.S. Pat. No. 4,891,227. The compositions useful herein can also be incorporated into and delivered from a soft-tipped or flexible dispensing device. These devices are useful for the controlled delivery of the compositions to the skin surface and have the advantage that the treatment composition itself never need be directly handled by the user. Nonlimiting examples of these devices comprise a fluid container including a mouth, an applicator, means for holding the applicator in the mouth of the container, and a normally closed pressure-responsive valve for permitting the flow of fluid from the container to the applicator upon the application of pressure to the valve. The valve can include a diaphragm formed from an elastically fluid impermeable material with a plurality of non-intersecting arcuate slits therein, where each slit has a base which is intersected by at least one other slit, and where each slit is out of intersecting relation with its own base, and wherein there is a means for disposing the valve in the container inside of the applicator. Examples of these applicator devices are described in U.S. Pat. No. 4,693,623; U.S. Pat. No. 4,620,648; U.S. Pat. No. 3,669,323; U.S. Pat. No. 3,418,055; and U.S. Pat. No. 3,410,645. Examples of applicators useful herein are commercially available from Dab-O-Matic, Mount Vernon, N.Y.

For example, in one embodiment, halofuginone is formulated for topical administration as a cream containing 0.03% halofuginone in a paraffin/water base daily for 60 days. (See e.g., Nagler and Pines, Transplantation, vol. 68(11):1806-9 (1999)). In the formulations for topical administration, halofuginone is present in an amount between 0.01% and 100% of the total composition. For example, the dosage of halofuginone is in the range of 0.01% to 50%; 0.01% to 25%; 0.01% to 10%; 0.01% to 5%; 0.01% to 2%; 0.01% to 1.5%; 0.01% to 1%; 0.01% to 0.75%; 0.01% to 0.5%; 0.01% to 0.25%; 0.01% to 0.1%; 0.01% to 0.09%; 0.01% to 0.08%; 0.01% to 0.07%; 0.01% to 0.06%; 0.01% to 0.05%; 0.01% to 0.04%; 0.01% to 0.03%; and 0.01% to 0.02%. As described above, the effect of the halofuginone cream is evaluated by photography/visual inspection by a plastic surgeon.

The invention is further described in the following examples, which do not limit the scope of the invention described in the claims.

### EXAMPLES

### Example 1

### Toxicity of Halofuginone

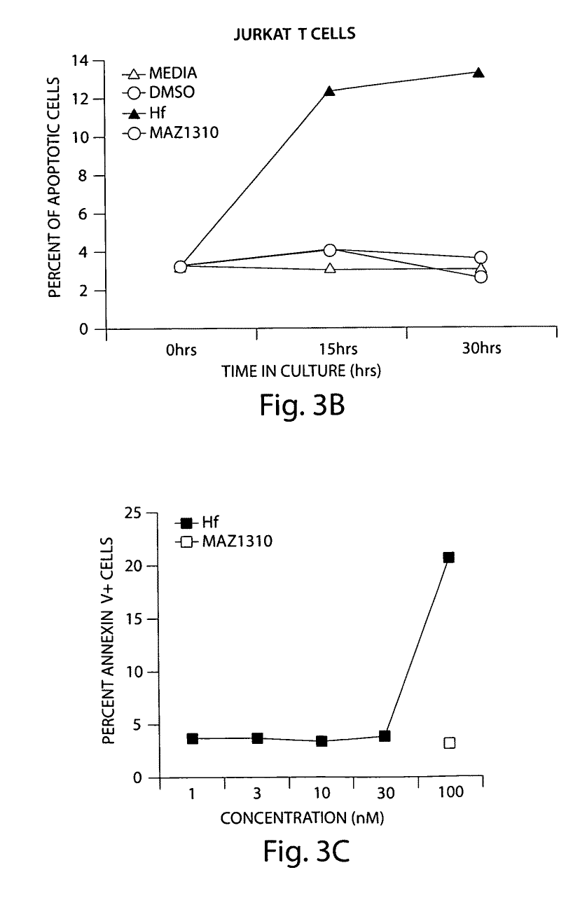

The non-specific cytoxicity of HF in both normal and transformed T-cells at high doses was evaluated. Anti-CD3/anti-CD28 activated primary T cells were harvested 6 days after activation and cultured in the presence of IL-2 with or without HF (100 nM). As a positive control, some T cells were cultured without IL-2 to induce apoptosis (FIG. 3A). Jurkat T cells, a transformed T cell leukemia line, were cultured in complete medium with or without HF (100 nM) as indicated (FIG. 3B). Both primary and Jurkat T cells were cultured for 30 hours and programmed cell death, i.e. apoptosis, was determined by Annexin V staining and propidium iodide (PI) uptake and cells were analyzed by flow cytometry. The percent of apoptotic T cells at each time point was plotted and was defined as Annexin V⁺ PI⁻. In both instances, 100 nM HF treatment caused significant apoptosis in both primary and Jurkat T cells. In further experiments, titrating amounts of HF were added to primary T cells and apoptosis was determine as above at the indicated time points. At 100 nM HF, but not 30 nM or lower, generalized T-cell apoptosis was observed (FIG. 3C).

The observed specific effects of HF on fibroblasts, e.g. maturation to myofibroblasts, contractility on collagen matrix, are not expected to have any detrimental effects on intact skin. While these effects could alter the kinetics of wound healing in damaged skin, topical HF has been shown in animal models to facilitate wound healing (e.g. Abramovitch et al.).

At concentrations over 80 nM, HF has a variety of non-specific effects on both T-cells and fibroblasts. In a crude preparation of primary T-cells, 80 nM HF was found to broadly suppress NFkB activation and T-cell secretion of cytokines (see e.g., Leiba et al., J Leukoc. Biol., vol. 80(2):399-406 (2006)). Studies were performed to reproduce these data and additionally find a generalized inhibition of T-cell proliferation at these concentrations. In fibroblasts, these studies, and others, found that concentrations of HF>80 nM inhibit proteins synthesis and cell proliferation in culture (McGaha et al., J Invest Dermatol., vol. 118(3):461-70 (2002)). Application of HF at doses up to 500 nM over a period of 5 days does not, however, cause death of cultured fibroblasts. In Xenopus feeding stage tadpoles, 400 nM HF does not have detectable toxic effects after treatment for 7 days. In mice, HF is commonly delivered IP at a dose of 1-5 μg/mouse without evident toxic effect. At this dose HF prevented radiation induced fibrosis but not normal healing of irradiated tissue (Xavier et al., J Biol Chem., vol. 279(15):15167-76 (2004)). In the mouse TSK model of dermal fibrosis, 1 μg HF/mouse delivered IP daily, had no toxic effects and reduced skin thickness of a TSK mouse to that of a normal mouse, but did not reduce normal skin thickness (McGaha et al.). In a human patient with Graft versus Host disease, daily topical application of 0.03% (˜500 μM) HF (Nagler and Pines, Transplantation, vol. 68(11):1806-9 (1999)) for a period of 6 months caused no local or systemic side effects, and HF was undetectable in serum throughout the course of treatment (consistent with observations in rabbits following topical treatment with HF at doses as high as 1%). In an oral phase I trial in humans, the dose limiting toxicity was nausea and vomiting, which occurred at 3.5 mg/day (peak plasma concentration of 3 ng/ml=˜8 nM). At the recommended tolerated dose for chronic treatment (0.5 mg/day), plasma levels of HF were ˜0.5 ng/ml (1 nM). (de Jonge et al., Eur J Cancer, vol. 42(12):1768-74 (2006)).

In T-cells and fibroblasts in vitro, HF has highly specific effects on pro-inflammatory and pro-fibrotic gene expression in a dose range of ˜2-40 mM. At doses >80 nM, non-specific effects on protein synthesis and cell proliferation are seen. The compositions are formulated to deliver a dosage of HF that will have specific effects on pro-inflammatory cytokine expression or ECM architecture in the 2-40 nM range, but keep the dosage below levels that can cause non-specific effects. The relationship between topical dose and concentration in skin has not been reported; topical HF has been reported in both animals and humans to not be detectable systemically.

### Example 2

### Specific Inhibition of Th17 Development and/or Expansion by Halofuginone

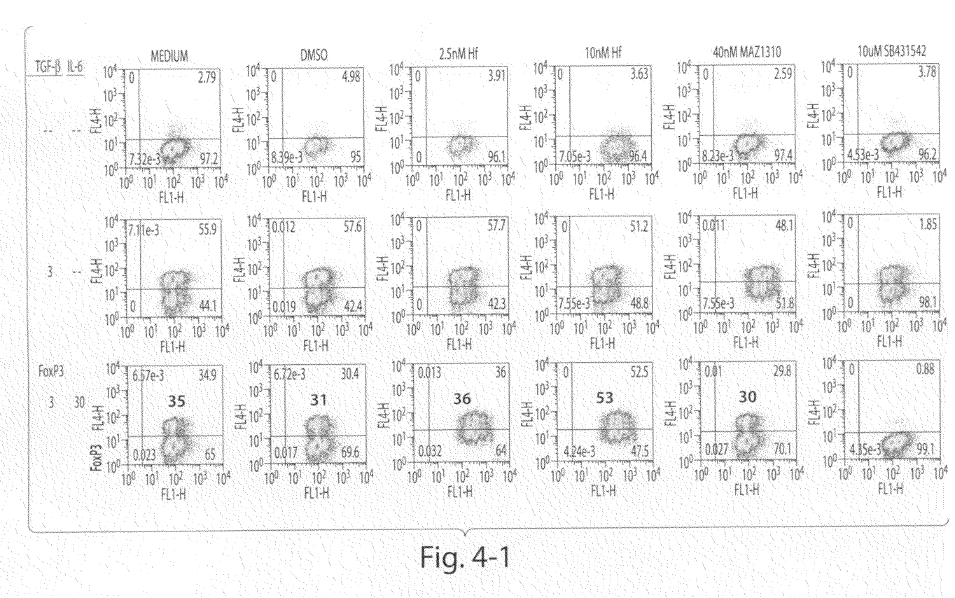

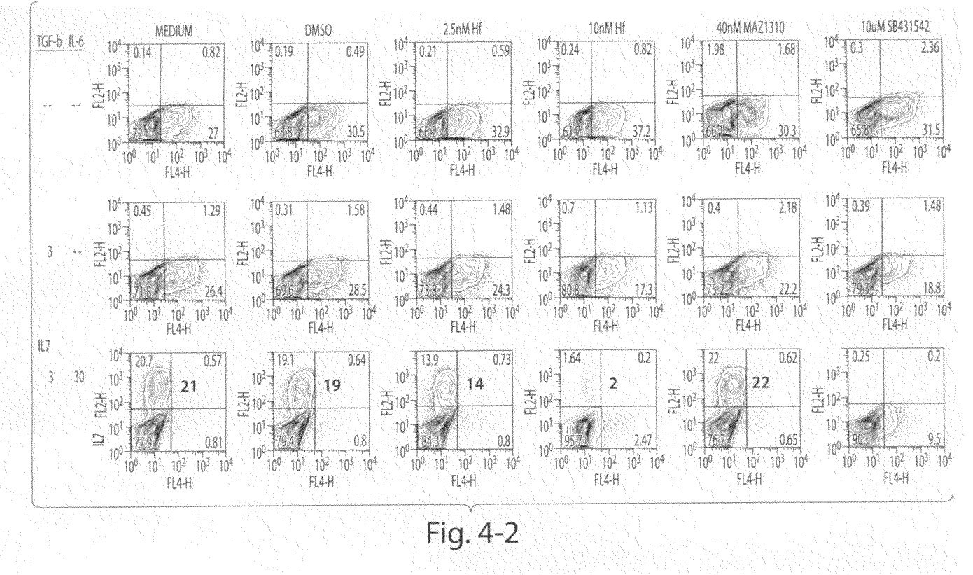

Low doses of halofuginone (HF) were tested to determine the ability of HF to enhance Treg differentiation, while suppressing Th17 differentiation. Naïve CD4+ T cells (CD4+ CD25−) were isolated from the spleen and peripheral lymph nodes of C57B/6 mice. T cells were then activated using anti-CD3 (0.3 μg/ml) and anti-CD28 (0.5 μg/ml) antibodies either in media alone (row 1, 4), TGFβ alone (3 ng/ml—row 2, 5) or TGFβ (3 ng/ml) plus IL-6 (30 ng/ml) (row 3, 6). T cells activated in each cytokine condition were further treated with either 2.5 nM or 10 nM of HF, 40 nM of MAZ1310 (inactive derivative of HF) or 10 μM of the type 1 TGFβ receptor kinase inhibitor SB431542. T cells were cultured for 3 days and CD25 and Foxp3 expression (Treg marker genes) was determine by FACS staining and flow cytometric analyses. Simultaneously, T cells were harvested and re-stimulated for 4 hours using phorbol myristic acetate (PMA; 10 nM), Ionomycin (1 μM) and cytokine secretion was prevented using brefeldin A (20 μg/ml). Following stimulation, the production of IL-17 and IFNγ were determined by intracellular staining and analyzed via flow cytometry. As shown in the plots in FIG. 4, 2.5-10 nM HF increased expression of the Treg marker gene FoxP3 in TGFβ/IL-6 treated T-cells concomitantly with inhibiting expression of IL-17, a marker of Th17 differentiation. The data shown in FIG. 5 was derived from experiments similar to those shown in FIG. 4.

The non-specific effects of HF on B and CD8+ T cells were evaluated. B220+B cells or CD8+ T cells were isolated from the spleen and peripheral lymph nodes of C57B/6 mice. B cell proliferation and IL-6 production was induced by culturing the cells with LPS (0.5 μg/ml) for 4 days. The effects of HF on B cell function was determined by adding titrating amounts of HF (1.25 nM-40 nM) at the time of LPS stimulation. B cell proliferation was monitored by labeling cells with Carboxyfluorescein diacetate, succinimidyl ester (CFSE; 5 μM) prior to LPS stimulation and determining the rate of CFSE dilution after 4 days by flow cytometric analyses. LPS-induced IL-6 production by B cells was determined by intracellular staining on day 4, following PMA+Ionomycin+Brefeldin A re-stimulation as described above. For CD8+ T cell experiments, purified CD8+ T cells were labeled with CFSE as above and the T cells were activated using anti-CD3 (1 μg/ml) and anti-CD28 (1 μg/ml) antibodies. Titrating amounts of HF (1.25 nM-40 nM) or MAZ1310 (40 nM) were added to the cultures at the time of activation and the T cell cultures were carried out for 4 days. CD8+ T cell proliferation was determined by monitoring CFSE dilution on day 3-post activation; CD25 expression was determined by FACS staining on day 2 of culture; differentiation of CD8+ T cells into cytolytic T cells (CTL) was evaluated on day 4 by determining expression of IFNγ and granzyme B following PMA+Ionomycin+Brefeldin A re-stimulation. CTLs, as shown in FIG. 6 were defined as IFNγ⁺ granzyme B⁺. In all experiments, cell proliferation and/or function was normalized to control (MAZ1310)-treated cells. As shown in FIG. 6, HF had little or no effect on B and CD8+ T cell function or proliferation at doses lower than 10 nM. At doses greater than 20 nM, however, HF-treated B and CD8+ T cells were profoundly impaired with respect to their proliferation and function (FIG. 6).

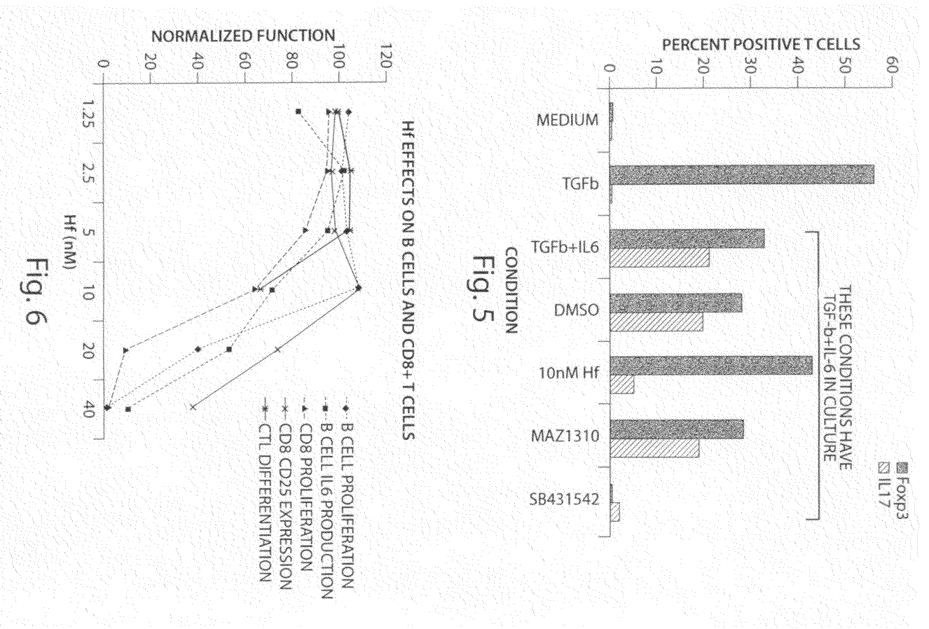

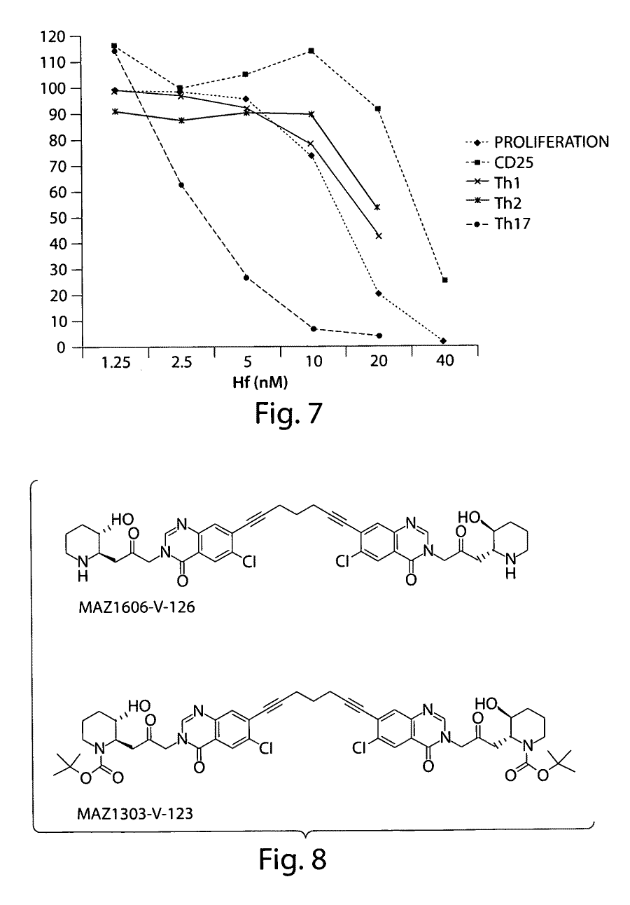

The dose-response of HF on CD4+ T cell proliferation, activation (i.e. CD25 upregulation) and differentiation to Th1, Th2 and Th17 lineages were evaluated. Purified murine naïve CD4+ CD25− T cells were activated using anti-CD3 (0.3 μg/ml) and anti-CD28 (0.5 μg/ml) antibodies and varying amounts of HF was added to each T cell culture condition (1.25 nM-40 nM). CD4+ T cell proliferation was monitored by CFSE dilution on day 3-post activation and CD25 expression was evaluated by FACS staining 2 days after activation. For T cell differentiation experiments, naïve T cells were activated as above in the presence of Th1 (IL-12 (20 ng/ml) plus anti-IL-4 (1.5 μg/ml)), Th2 (IL-4 (50 ng/ml) plus anti-IFNγ (0.5 mg/ml)) or Th17 (TGFβ (3 ng/ml) plus IL-6 (30 ng/ml)) polarizing conditions plus titrating amounts of HF (1.25 nM-40 nM) or MAZ1310 (40 nM). The percentage of Th17 cells, (IL-17⁺ IFNγ⁻) was evaluated on day 3 following activation, whereas the abundance of Th1 cells (IFNγ⁺ IL-4) and Th2 cells (IL-4⁺ IFNγ⁻) was determined 5 days following activation. All cytokine expression was determined by intracellular staining following PMA+Ionomycin+Brefeldin A re-stimulation and all data was normalized to control (MAZ1310)-treated cells. As shown in FIG. 7, the effects of HF on Th17 differentiation were seen at ˜5- to 10-fold lower doses compared to those which lead to general inhibition of T-cell proliferation and differentiation to Th1 or Th2 lineages and those effects that were observed in previous studies, e.g., in Lieba et al., J. Leuk. Biol., vol. 80:1-8 (2006).

### Example 3

### Materials and Methods

Mice: Mice were housed in specific pathogen-free barrier facilities and were used in accordance with protocols approved by the animal care and use committees of the Immune Disease Institute and Harvard Medical School. Wild-type C57B/6 mice were purchased from Jackson laboratories (Bar Harbor, Me.) and were used for all in vitro culture experiments unless otherwise noted. ROSA26-YFP^{fl/fl} mice have been described elsewhere. (Srinivas, S. et al. Cre reporter strains produced by targeted insertion of EYFP and ECFP into the ROSA26 locus. BMC Dev Biol 1, 4 (2001)). ROSA26-STAT3C-GFP^{fl/fl} mice were generated as described previously. (Mesaros, A. et al. Activation of Stat3 signaling in AgRP neurons promotes locomotor activity. Cell Metab 7, 236-48 (2008)). Spleens and peripheral lymph nodes from Foxp3^{gfp} and Foxp3^{ko} mice were generated as previously described. (Gavin, M. A. et al. Foxp3-dependent programme of regulatory T-cell differentiation. Nature 445, 771-5 (2007)).

Microarrays, data analyses and statistics: RNA prepared from activated T cells treated with 10 nM HF or MAZ1310 for either 3- or 6-hours, was amplified, biotin-labeled (MessageAmp II Biotin-Enhanced kit, Ambion, Austin, Tex.), and purified using the RNeasy Mini Kit (Qiagen, Valencia, Calif.). The resulting cRNAs were hybridized to M430 2.0 chips (Affymetrix, Inc.). Raw data were normalized using the RMA algorithm implemented in the “Expression File Creator” module from the GenePattern software package (Reich, M. et al. GenePattern 2.0. Nat Genet 38, 500-1 (2006)). Data were visualized using the GenePattern “Multiplot” modules. Gene expression distribution analyses were performed using Chi-squared statistical tests. For all other statistical comparisons, p values were generated using one-tailed student T-tests on duplicate or triplicate samples.

Cytokines, antibodies and T cell culture: Purified CD4⁺ CD25⁻ T cells were activated in vitro as previously described (I. M. Djuretic et al., Nat Immunol 8, 145 (2007)) using 0.3 μg/ml hamster anti-mouse CD3 (clone 145-2C11) (ATCC—Manassas, Va.) and 0.5 μg/ml hamster anti-mouse CD28 (BD Pharmingen, San Jose, Calif.). Activated cell cultures were differentiated using the following combinations of cytokines and antibodies: iTreg=recombinant human TGFβ1 (3 ng/ml; R&D systems, Minneapolis, Minn.), Th17=TGFβ1 (3 ng/ml) plus recombinant mouse IL-6 (30 ng/ml, R&D systems). Th1 and Th2 differentiation was performed as previously described. (I. M. Djuretic et al., Nat Immunol 8, 145 (2007)). Human IL-2 supernatant (National Cancer Institute) was used in culture at 0.01 U/ml and was added at 48 hours-post activation when T cells were split into tissue culture wells lacking CD3 and CD28 antibodies, with the exception of Th17 cultures that were maintained in the absence of IL-2. All reagents were added at the time of T cell activation and again at 48-hours post activation unless indicated otherwise. For some experiments, purified CD4⁺ CD25⁻ T cells, CD8⁺ T cells or B cells were labeled with 1 μM CFSE (Invitrogen) prior to activation in accordance with manufacturers instructions.

Inhibitors: 1 kg of 10% pure HF (Hangpoon Chemical Co., Seoul, Korea), was further purified via HPLC to >99% purity and used for experiments. MAZ1310 was generated by chemical derivatization of halofuginone and was used at equal concentrations as a negative control. HF and MAZ1310 were prepared as 100 mM stock solutions in DMSO and diluted to the indicated concentrations. SB-431542 (Tocris bioscience, Ellisville, Mo.) was prepared as a 10 mM stock solution in DMSO and was used in culture at 10 μM. L-tryptophanol was prepared as a 20 mM stock solution in 0.1 M NaOH, pH 7.4 and was used at 0.2 mM.

Amino acid starvation: T cells were activated and differentiated as above in D-MEM medium without L-cysteine and L-methionine (Invitrogen, Carlsbad, Calif.), or D-MEM medium without L-leucine. Stocks containing 20 mM L-cysteine (Sigma, St. Louis, Mo.) plus 10 mM L-methionine (Sigma), or 400 mM L-leucine (Sigma) were prepared in ddH₂O, pH 1.0 and added to medium at the indicated concentrations. L-tryptophanol was prepared as a 20 mM stock solution in 0.1 M NaOH, pH 7.4 and was added to complete medium at 0.2 mM.

Cell isolation: Primary murine T and B cells were purified by cell sorting. CD4⁺ CD25⁻ T cells were positively selected using CD4 dynabeads and detachabeads (Dynal, Oslo, Norway) per manufacturers instructions followed by Treg depletion using a CD25 microbead kit (Miltenyi biotech, Auburn, Calif.). Naïve (CD4⁺ CD62L^{hi} CD44^{lo} Foxp3^{gfp-} or CD4⁺ CD62L^{hi} CD44^{lo} CD25⁻) T cells were purified from Foxp3^{gfp} or Foxp3^{ko} mice, respectively, by FACS sorting. CD8⁺ T cells or B cells were isolated from CD4⁻ fractions using CD8 negative isolation kit (Dynal) or CD43 negative isolation kit (Miltenyi biotech), respectively. CD43-depleted B cells were activated in vitro by culturing with 25 μg/ml LPS (Sigma, St. Louis, Mo.) for 3-4 days in the presence or absence of TGFβ. HF or MAZ1310 was added at the time of LPS stimulation and again at day 3.

Tat-Cre transduction: 6× His-TAT-NLS-Cre (HTNC—herein called “TAT-Cre”) was prepared as previously described (M. Peitz, et al., Proc Natl Acad Sci USA 99, 4489, 2002). Purified T cells where rested in complete medium for 30 minutes, washed 3 times in ADCF-Mab serum free medium (Hyclone, Logan, Utah) and resuspended in pre-warmed serum free medium supplemented with 50 μg/ml of TAT-Cre. Following a 45 minute incubation at 37° C., TAT-Cre transduction was stopped using media containing 10% FCS and T cells were rested for 4-6 hours in complete medium prior to activation.

Human T cell isolation and activation: Resting CD4⁺ T cells were isolated from PBMC of healthy human donors using Dynal CD4 Positive Isolation Kit (Invitrogen, Carlsbad, Calif.) as previously described (M. Sundrud et al., Blood 106, 3440, 2005). CD4⁺ cells were further purified to obtain memory T cells by staining with PE-conjugated anti-human CD45RO-PE antibodies (BD Biosciences), and sorting on a FACSAria cytometer (BD Biosciences). Following purification, cells were greater than 99% CD4⁺ CD45RO⁺. CD14⁺ monocytes were isolated from autologous PBMC by MACS sorting using a magnetic separator (AutoMACS, Miltenyi Biotech) and were more then 99% pure following isolation. T cell activation was performed by plating purified monocytes in a 96-well flat bottom plate at a concentration of 20,000 cells per well in complete medium overnight. 105 purified human memory T cells were added to monocyte cultures in the presence of soluble anti-CD3/anti-CD28 beads (Dynabeads, Invitrogen). T cells were expanded in the presence HF or MAZ1310 as indicated for 6 days and intracellular cytokine expression was determined by intracellular staining.

Retroviral transductions: MIG and MIG.RORγt retroviral cDNA were gifts from Dr. Dan Littman (I. Ivanov, et al., Cell 126, 1121, 2006). pRV and pRV.FOXP3 retroviral constructs have been described previously (Y. Wu et al., Cell 126, 375, 2006). Retroviral particles were generated using the phoenix-Eco system (ATCC). Supernatants were concentrated by centrifugation and stored at −80° C. prior to use in culture. Thawed retroviral supernatants were added to T cell cultures 12 hours after T cell activation in the presence of 8 μg/ml polybrene (American bioanalytical, Natick, Mass.) and centrifuged for 1 hour at room temperature to enhance infections.

Detection of cytokine production: Cytokines secreted into media supernatant were measured using the mouse Th1/Th2 cytometric bead array (CBA, BD Pharmingen) in accordance with manufacturers instructions. Briefly, CD4⁺ CD25⁻ T cells were activated in anti-CD3/anti-CD28-coated tissue culture wells and supernatants were collected at the indicated times. For detection of intracellular cytokines in murine cells, cultured T or B cells were stimulated with 10 nM PMA (Sigma) and 1 mM ionomycin (Sigma) for 4-5 hours in the presence of 10 mM brefeldin A (Sigma). Stimulated cells were harvested, washed with PBS and fixed with PBS plus 4% paraformaldehyde at room temperature for 20 minutes. Cells were then washed with PBS, permeabilized with PBS supplemented with 1% BSA and 0.5% saponin (Sigma) at room temperature for 10 minutes before cytokine-specific antibodies were added and incubated with cells for an additional 20 minutes at room temperature. Human T cells were restimulated with PMA (20 ng/ml) (Sigma) and Ionomycin (500 ng/ml) (Sigma) for 6 hours in the presence of golgi plug (BD Biosciences) and intracellular staining was performed using cytofix/cytoperm kit (BD Biosciences) per manufacturers instructions. All stained cells were stored at 4° C. in PBS plus 1% paraformaldehyde prior to FACS analyses.

FACS analyses and sorting: All cell surface staining was performed in FACS buffer (PBS/2% FCS/0.1% NaN₃) and antibodies were incubated with cells on ice for 20-30 minutes. Cells were washed with FACS buffer and fixed with FACS buffer plus 1% paraformaldehyde prior to data acquisition. For phospho-STAT3 intracellular staining, stimulated T cells cultured with or without TGFβ plus IL-6 for the indicated times were harvested on ice and fixed in PBS plus 2% paraformaldehyde for 10 minutes at 37° C. Fixed cells were washed twice with staining buffer (PBS/1% BSA/0.1% NaN₃) and then permeabilized with perm buffer III (BD Pharmingen) on ice for 30 minutes. Cells were then washed twice with staining buffer and PE-conjugated anti-STAT3 (pY705) (BD Pharmingen) was added per manufacturers instructions and incubated with cells at room temperature for 45-60 minutes. Cells were then washed and stored in staining buffer prior to data acquisition. Foxp3 intracellular staining was performed using a Foxp3 intracellular staining kit (eBioscience, San Diego, Calif.) in accordance with manufacturers instructions. Fluorescent-conjugated antibodies purchased from BD Pharmingen were percp-Cy5.5-conjugated anti-CD4, PE-conjugated anti-CD25, PE-conjugated anti-IL-17, PE-conjugated anti-phospho-STAT3 and APC-conjugated anti-human IFNγ. Fluorescent-conjugated antibodies purchased from eBiosciences include FITC-conjugated anti-CD8, APC-conjugated anti-mouse/rat Foxp3, PE-conjugated anti-IL-4, APC-conjugated anti-IFNγ, PE-conjugated anti-granzyme B, APC-conjugated streptavidin, PE-conjugated anti-IL-6 and PE-conjugated anti-human IL-17. Biotin-conjugated anti-IgA antibody was purchased from Southern biotech (Birmingham, Ala.). All FACS data was acquired on a FACSCalibur flow cytometer (BD Pharmingen) and analyzed using FlowJo software (Treestar, Inc., Ashland, Oreg.). FACS sorting was performed on a FACS-Diva cytometer (BD Pharmingen).

Quantitative real-time PCR: T cells were activated as described above, collected at the indicated times and pellets were flash-frozen in liquid nitrogen. Total RNA was obtained by RNeasy (Quiagen, Valencia, Calif.) column purification per manufacturers instructions. RORγt expression was determined after reverse transcription using the message sensor kit (Ambion, Austin, Tex.) per manufacturers instructions and taqman primers and probe as described elsewhere (I. Ivanov, et al., Cell 126, 1121, 2006). Sybrgreen quantitative real-time PCR was performed on T cell RNA samples following reverse transcription via SuperScript II first-strand cDNA synthesis kit (Invitrogen, Carlsbad, Calif.). All PCR data was collected on an iCycler thermal cycler (Biorad, Hercules, Calif.). Primer sequences used for detecting stress response genes are listed below.

| | |
|---|---|
| Asns forward: | |
| 5′-TGACTGCCTTTCCGTGCAGTGTCTGAG-3′, | (SEQ ID NO: 1) |
| Asns reverse: | |
| 5′-ACAGCCAAGCGGTGAAAGCCAAAGCAGC | (SEQ ID NO: 2) |
| Gpt2 forward: | |
| 5′- TAGTCACAGCAGCGCTGCAGCCGAAGC-3′ | (SEQ ID NO: 3) |
| Gpt2 reverse: | |
| 5′- TACTCCACCGCCTTCACCTGCGGGTTC-3′ | (SEQ ID NO: 4) |
| eIF4Ebp1 forward: | |
| 5′- ACCAGGATTATCTATGACCGGAAATTTC-3′ | (SEQ ID NO: 5) |
| eIF4Ebp1 reverse: | |
| 5′- TGGGAGGCTCATCGCTGGTAGGGCTAG-3′ | (SEQ ID NO: 6) |
| Hprt forward: | |
| 5′-GGGGGCTATAAGTTCTTTGCTGACC-3 | (SEQ ID NO: 7) |
| Hprt reverse: | |
| 5′-TCCAACACTTCGAGAGGTCCTTTTCAC-3′ | (SEQ ID NO: 8) |

Western blotting: Whole cell lysates were generated from T cells activated for the indicated times. For STAT3 and Smad2/3 western blots cells were harvested, washed in PBS and lysed in 50 mM Tris, pH 7.4, 0.1% SDS, 1% Triton-×100, 140 mM NaCl, 1 mM EDTA, 1 mM EGTA supplemented with protease inhibitors tablets (Roche—Germany), 1 mM NaF and 1 mM Na₃VO₄. For eIF2α and ATF4 western blots, cells were harvested as above and lysed in 50 mM Tris, pH 7.4, 2% SDS, 20% glycerol and 2 mM EDTA supplemented with protease and phosphatase inhibitors as above. All lysates were cleared via centrifugation and 15-30 μg of protein was resolved by SDS-PAGE. Protein was transferred to nitrocellulose membranes, blocked and blotted using specific antibodies. Antibodies used for western blot analysis were anti-phospho-Smad2, anti-STAT3 (pY705), anti-STAT3, anti-eIF2α^{pS51}, anti-eIF2α, anti-GCN2^{pT898}, anti-GCN2 (all from cell signaling technology, Danvers, Mass.). Anti-ATF4/CREB2 and anti-β-actin were purchased from Santa Cruz biotechnology (Santa Cruz, Calif.). HRP-conjugated secondary antibodies were all purchased from Sigma, with the exception of HRP-conjugated anti-armenian hamster antibody (Jackson Immunoresearch—West Grove, Pa.).

### Example 4

### Effect of Halofuginone on T Cell Differentiation and Effector Function

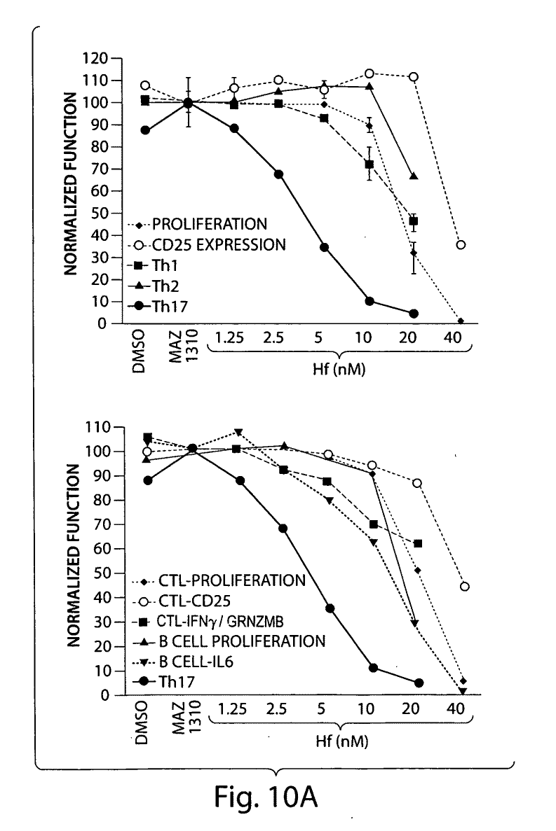

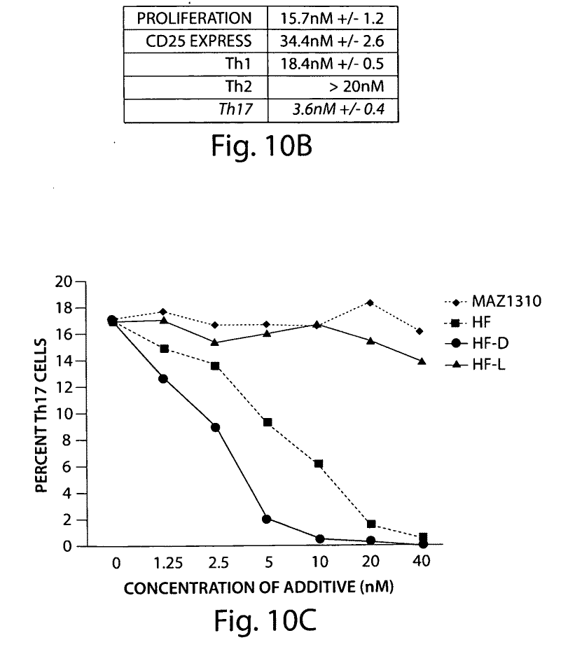

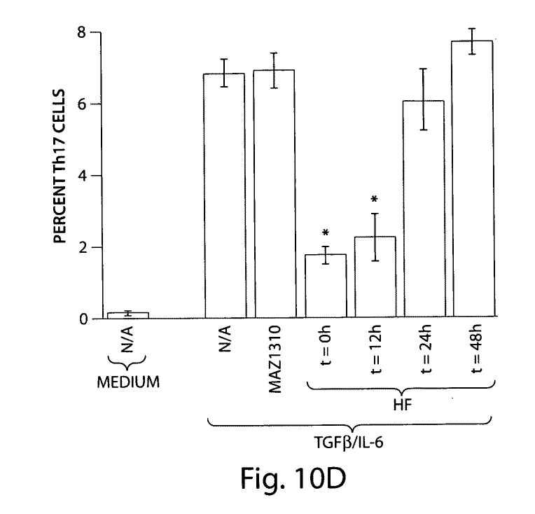

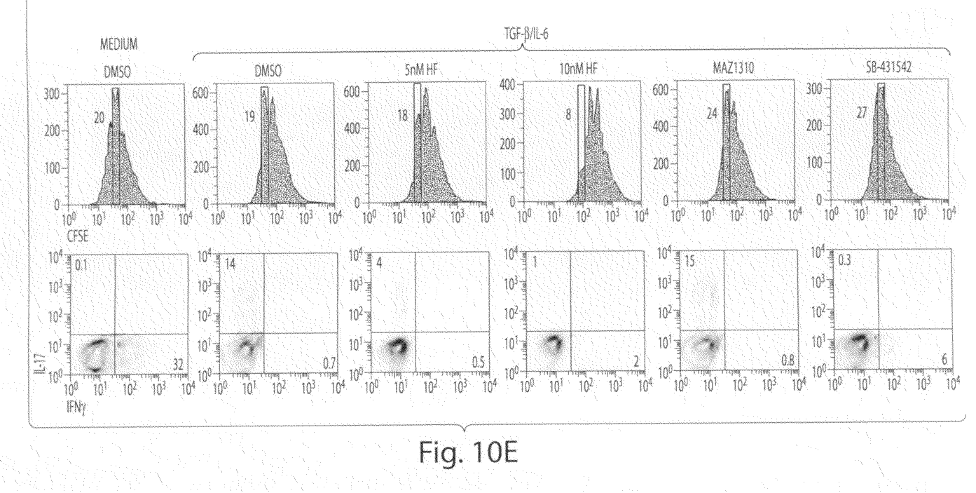

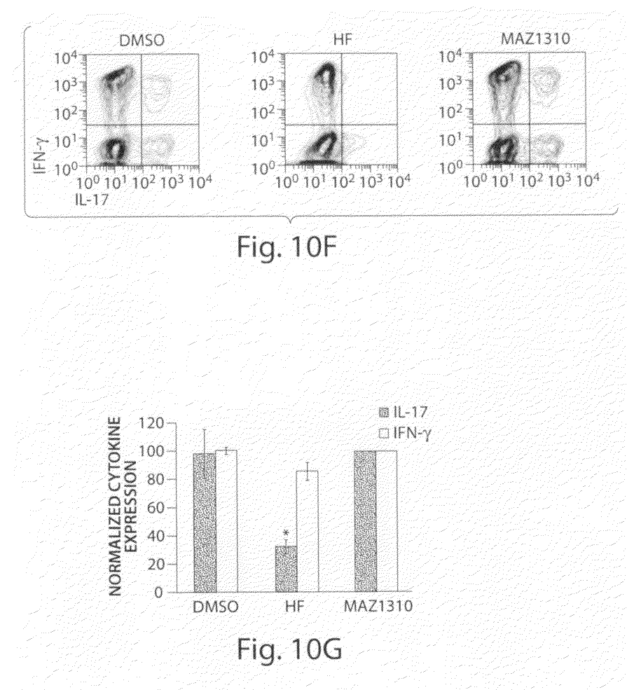

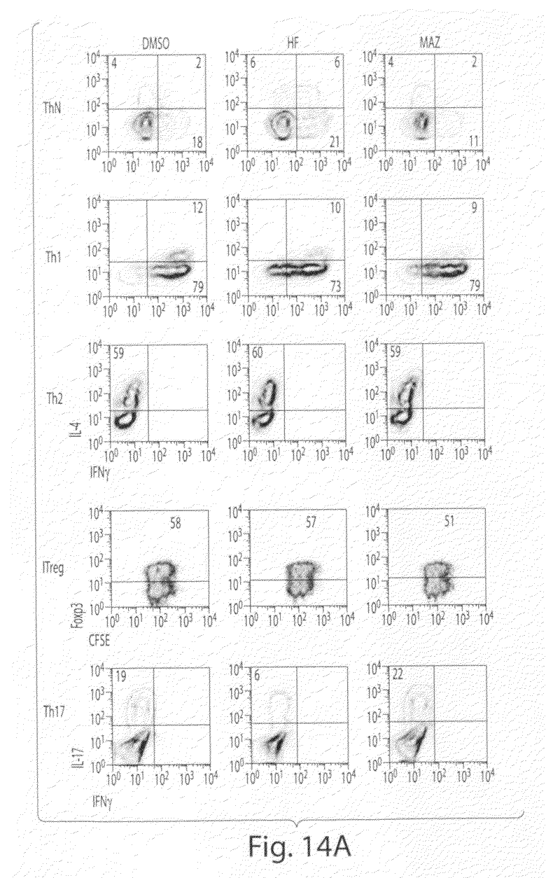

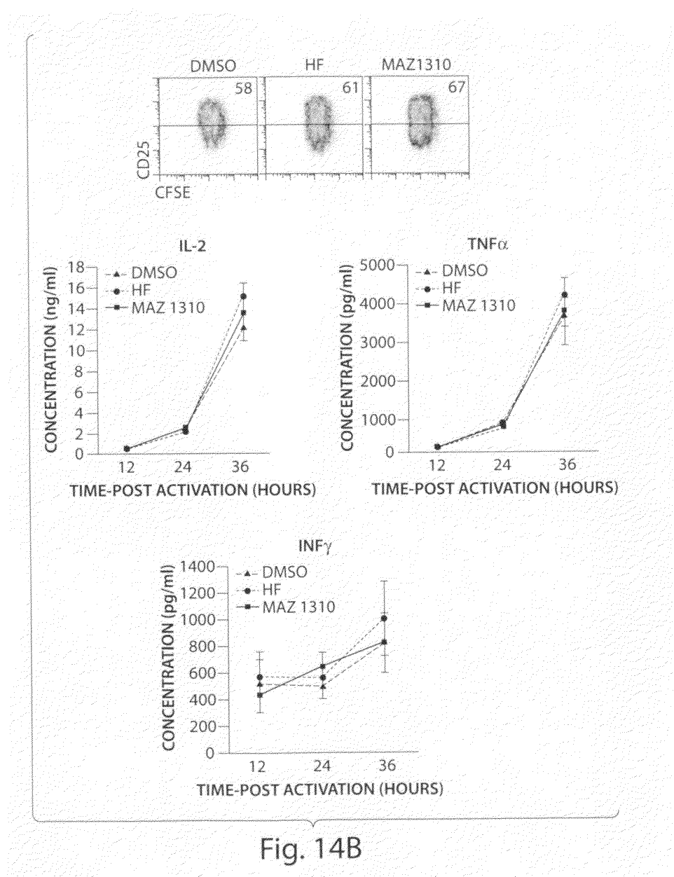

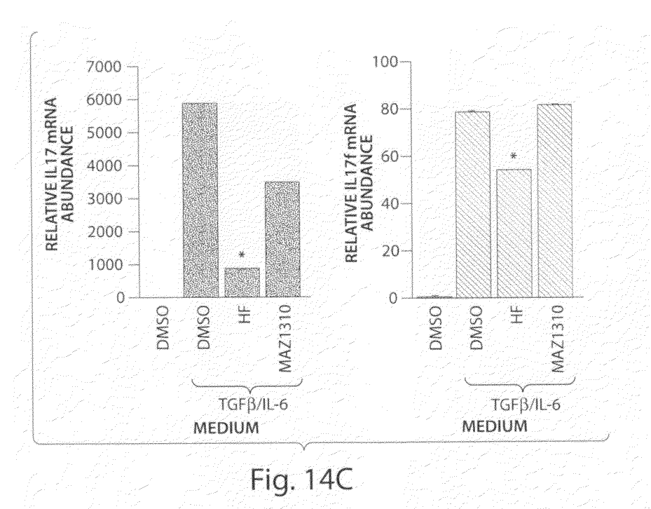

To investigate whether HF can modulate T cell differentiation or effector function, purified murine CD4⁺ CD25⁻ T cells were treated with HF or its inactive derivative MAZ1310 (FIG. 1B), and the cells were then stimulated in the absence or presence of polarizing cytokines to induce Th1, Th2, iTreg or Th17 differentiation. Dose-response experiments revealed a remarkably selective effect of HF on Th17 differentiation, assessed here as the percentage of IL-17⁺ IFNγ⁻ cells following restimulation on day 4-5. HF repressed Th17 differentiation in a dose-dependent manner with an IC₅₀ of 3.6 nM±0.4 nM (FIGS. 10A, 10B). Low concentrations of HF (1-10 nM) that strongly reduced IL-17 production (FIGS. 10A, 10B, and 14A) did not affect T cell proliferation, CD25 upregulation or production of IL-2, TNF or IFNγ (FIG. 14B). Low-dose HF also failed to modulate Th1, Th2 or iTreg differentiation as assessed by IFNγ, IL-4 or Foxp3 expression, respectively (FIG. 14A). At approximately 10-fold higher concentrations (>20 nM), HF induced a general inhibition of T and B cell activation, proliferation and effector function (FIGS. 10A, 10B), effects consistent with a previous report. (Leiba, M. et al. Halofuginone inhibits NF-kappaB and p38 MAPK in activated T cells. J Leukoc Biol 80, 399-406 (2006)). The selective inhibition of Th17 differentiation by low-dose HF was stereospecific: the HPLC-purified D-enantiomer of HF inhibited IL-17 expression more potently than a racemic mix, whereas the L-enantiomer was completely inactive (FIG. 10C).

Inhibition of IL-17 expression was most pronounced when HF was added during a 12-hour window at the start of the culture period (FIG. 10D) and HF treatment impaired expression of both Il17 and Il17f mRNA (FIG. 14C). These results indicate that HF regulates early events, such as, for example, being involved in Th17 lineage commitment, rather than influencing the expansion of Th17 cells or preventing acute cytokine expression upon restimulation. Inhibition by HF was not due to perturbation of cell cycle progression or selective survival; HF inhibited IL-17 expression in a dose-dependent manner even when only cells that had completed an equivalent number of cell divisions as judged by CFSE dilution were considered (FIG. 10E). HF also reduced IL-17 expression in cultures where IFNγ and IL-4, cytokines known to inhibit Th17 differentiation (Park, H. et al. A distinct lineage of CD4 T cells regulates tissue inflammation by producing interleukin 17. Nat Immunol 6, 1133-41 (2005)) were blocked by addition of neutralizing antibodies. Thus, HF-mediated inhibition of Th17 cell development is not secondary to effects on T cell proliferation or auxiliary cytokine production.

In light of recent reports that IL-17 expression may be differentially regulated in murine versus human T cells (see e.g., Manel, N., Unutmaz, D. & Littman, D. R. The differentiation of human T(H)-17 cells requires transforming growth factor-beta and induction of the nuclear receptor RORgammat. Nat Immunol 9, 641-9 (2008); Wilson, N. J. et al. Development, cytokine profile and function of human interleukin 17-producing helper T cells. Nat Immunol 8, 950-7 (2007); and Acosta-Rodriguez, E. V., Napolitani, G., Lanzavecchia, A. & Sallusto, F. Interleukins 1 beta and 6 but not transforming growth factor-beta are essential for the differentiation of interleukin 17-producing human T helper cells. Nat Immunol 8, 942-9 (2007)), studies were designed to evaluate whether HF would also modulate IL-17 expression by human CD4⁺ T cells. These experiments showed that HF treatment greatly reduced both the percentage of human T cells expressing IL-17 and the amount of IL-17 produced (FIGS. 10F, 10G). In striking contrast, IFNγ expression was essentially unaffected by HF treatment (FIGS. 10F, 10G). Therefore, HF selectively limits IL-17 expression in both human and mouse T cells.

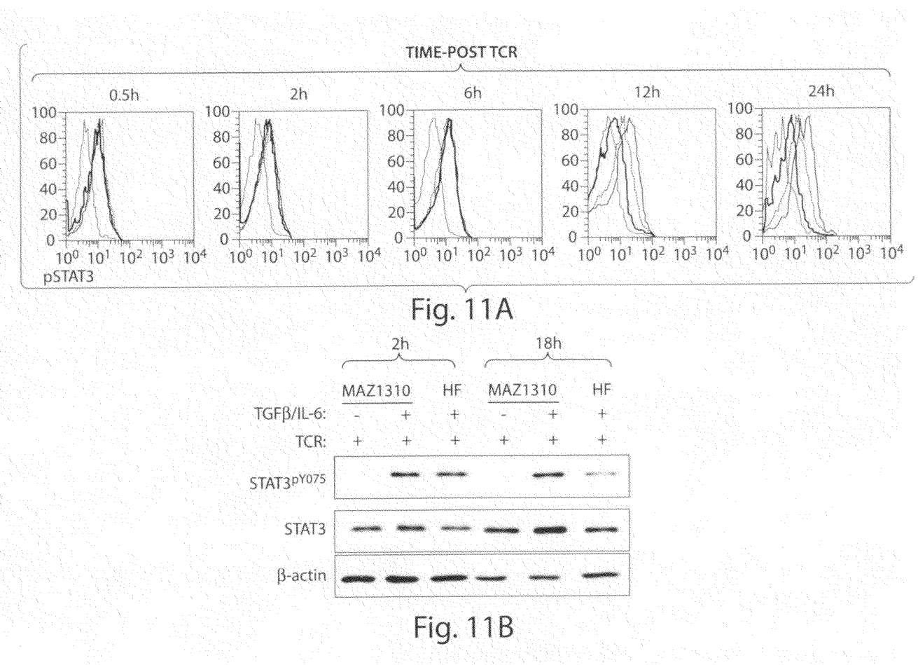

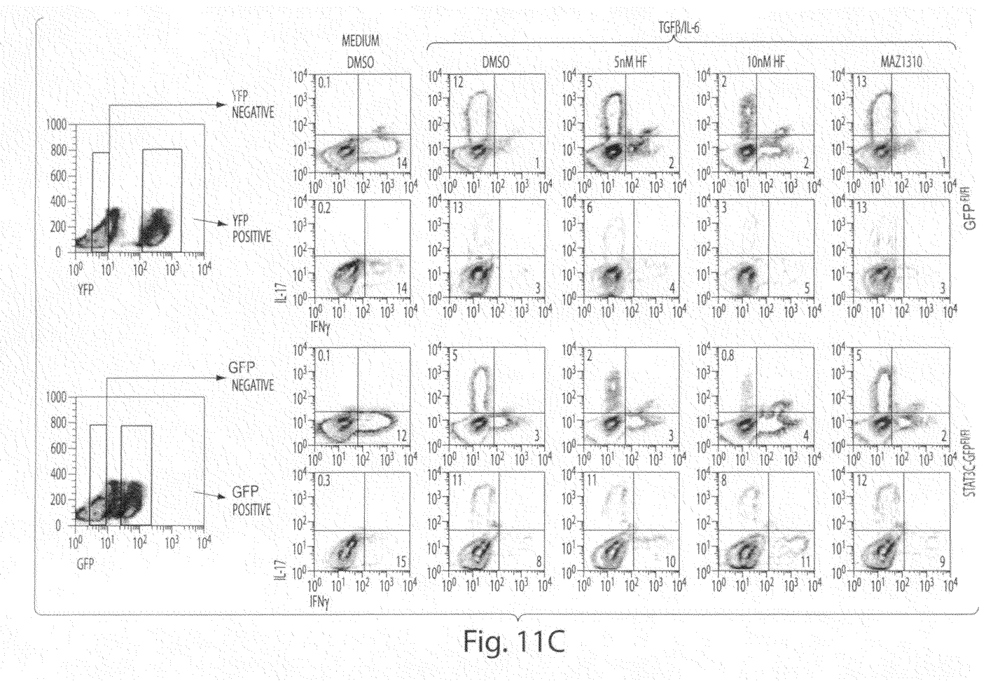

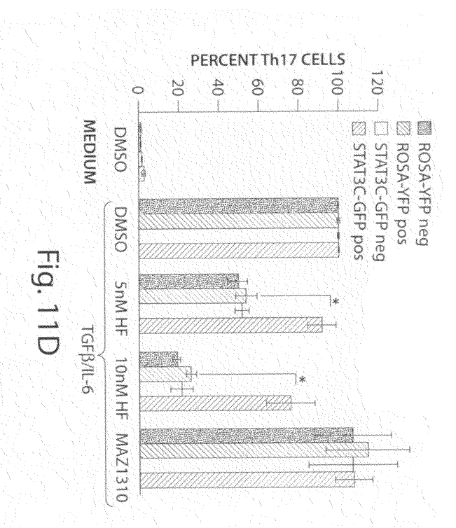

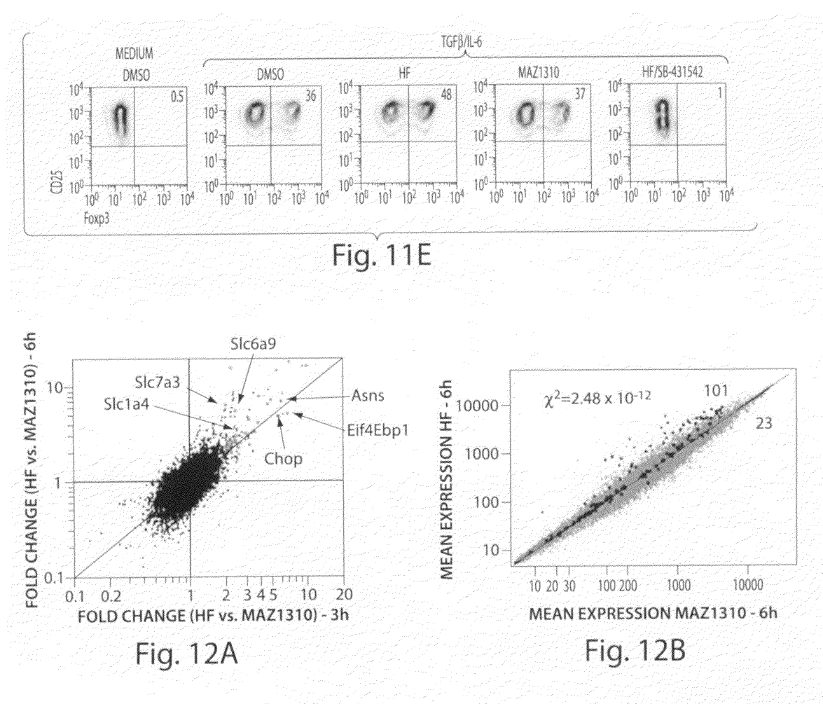

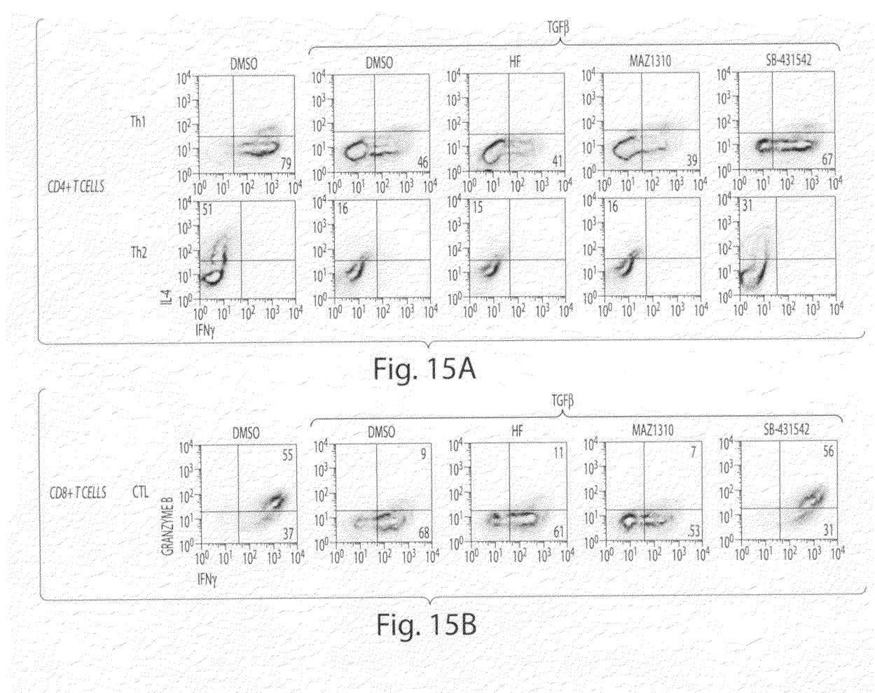

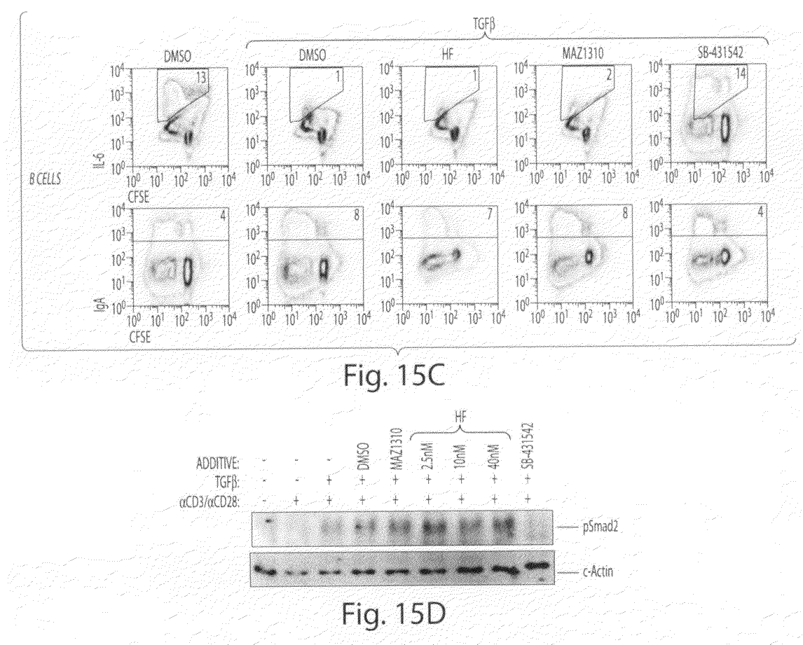

Th17 differentiation is synergistically regulated by TGFβ and by the pro-inflammatory cytokines IL-6 and IL-21. (Zhou, L. et al. IL-6 programs T(H)-17 cell differentiation by promoting sequential engagement of the IL-21 and IL-23 pathways. Nat Immunol 8, 967-74 (2007); Wei, L., Laurence, A., Elias, K. M. & O'Shea, J. J. IL-21 is produced by Th17 cells and drives IL-17 production in a STAT3-dependent manner. J Biol Chem 282, 34605-10 (2007); Nurieva, R. et al. Essential autocrine regulation by IL-21 in the generation of inflammatory T cells. Nature 448, 480-3 (2007); Veldhoen, M., Hocking, R. J., Atkins, C. J., Locksley, R. M. & Stockinger, B. TGFbeta in the context of an inflammatory cytokine milieu supports de novo differentiation of IL-17-producing T cells. Immunity 24, 179-89 (2006); Ivanov, I I et al. The orphan nuclear receptor RORgammat directs the differentiation program of proinflammatory IL-17+T helper cells. Cell 126, 1121-33 (2006); Bettelli, E. et al. Reciprocal developmental pathways for the generation of pathogenic effector TH17 and regulatory T cells. Nature 441, 235-8 (2006); and Yang, X. O. et al. STAT3 regulates cytokine-mediated generation of inflammatory helper T cells. J Biol Chem 282, 9358-63 (2007)). Despite prior reports that HF can attenuate TGFβ signaling (Gnainsky, Y. et al. Gene expression during chemically induced liver fibrosis: effect of halofuginone on TGF-beta signaling. Cell Tissue Res 328, 153-66 (2007); and Flanders, K. C. Smad3 as a mediator of the fibrotic response. Int J Exp Pathol 85, 47-64 (2004)), it was found that HF inhibited neither TGFβ-induced Smad phosphorylation nor a variety of other lymphocyte responses to TGFβ (Li, M. O., Wan, Y. Y., Sanjabi, S., Robertson, A. K. & Flavell, R. A. Transforming growth factor-beta regulation of immune responses. Annu Rev Immunol 24, 99-146 (2006); Glimcher, L. H., Townsend, M. J., Sullivan, B. M. & Lord, G. M. Recent developments in the transcriptional regulation of cytolytic effector cells. Nat Rev Immunol 4, 900-11 (2004); and van Vlasselaer, P., Punnonen, J. & de Vries, J. E. Transforming growth factor-beta directs IgA switching in human B cells. J Immunol 148, 2062-7 (1992)), in contrast to the type 1 TGFβ receptor kinase inhibitor SB-431542 (Inman, G. J. et al. SB-431542 is a potent and specific inhibitor of transforming growth factor-beta superfamily type I activin receptor-like kinase (ALK) receptors ALK4, ALK5, and ALK7. Mol Pharmacol 62, 65-74 (2002)), which abrogated all responses to TGFβ (FIG. 15). Since STAT3 is the major transducer of IL-6 and IL-21 action, studies where then designed to examine the kinetics of STAT3 phosphorylation in HF-treated T cells. HF did not interfere with STAT3 activation during the first 6 hours of Th17 differentiation, but rather decreased the maintenance of STAT3 phosphorylation beginning around 12 hours-post activation (FIGS. 11A, 11B). Studies were then designed to evaluate whether inhibition of Th17 differentiation by HF would be restored by transgenic expression of a hyperactive STAT3 protein (STAT3C). (Bromberg, J. F. et al. Stat3 as an oncogene. Cell 98, 295-303 (1999)). T cells isolated from homozygous mice containing a floxed stop-STAT3C-IRES-EGFP (STAT3C-GFP^{fl/fl}) (Mesaros, A. et al. Activation of Stat3 signaling in AgRP neurons promotes locomotor activity. Cell Metab 7, 236-48 (2008)) or stop-YFP (YFP^{fl/fl}) (Srinivas, S. et al. Cre reporter strains produced by targeted insertion of EYFP and ECFP into the ROSA26 locus. BMC Dev Biol 1, 4 (2001)) cassette inserted into the ROSA26 locus were transduced with a cell-permeant TAT-Cre fusion protein (Peitz, M., Pfannkuche, K., Rajewsky, K. & Edenhofer, F. Ability of the hydrophobic FGF and basic TAT peptides to promote cellular uptake of recombinant Cre recombinase: a tool for efficient genetic engineering of mammalian genomes. Proc Natl Acad Sci USA 99, 4489-94 (2002)) to delete the stop cassette and these cells were activated in the presence of TGFβ plus IL-6, with either HF or MAZ1310. As expected, HF strongly impaired Th17 differentiation of cells expressing YFP or those not expressing a transgene (FIG. 11C, top three panels); in contrast, T cells expressing STAT3C (defined by their concomitant expression of GFP) remained capable of differentiating into Th17 cells even in the presence of 10 nM HF (FIG. 11C, bottom panel). Data from multiple experiments are quantified and summarized in FIG. 11D. Collectively, these results demonstrate that HF inhibits Th17 differentiation through its ability to prevent sustained activation of STAT3.

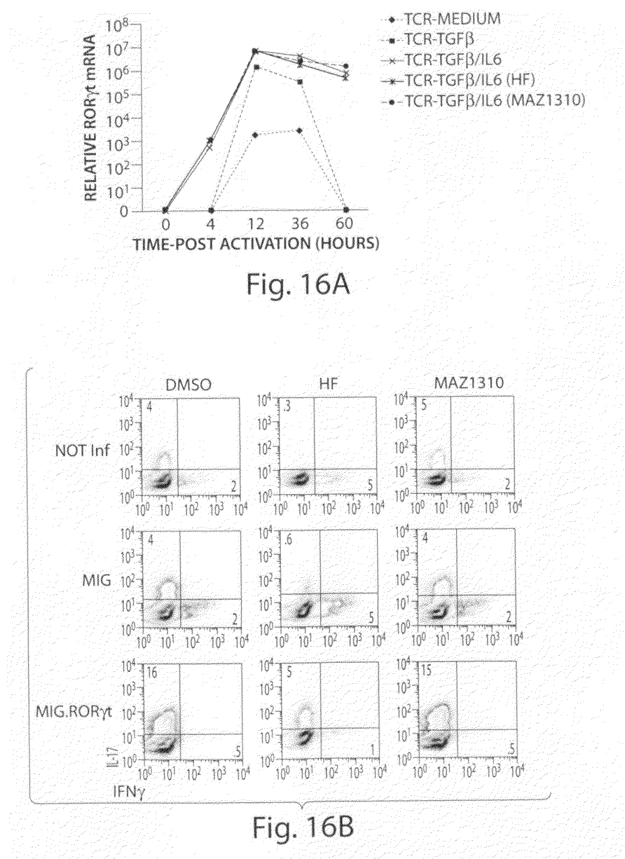

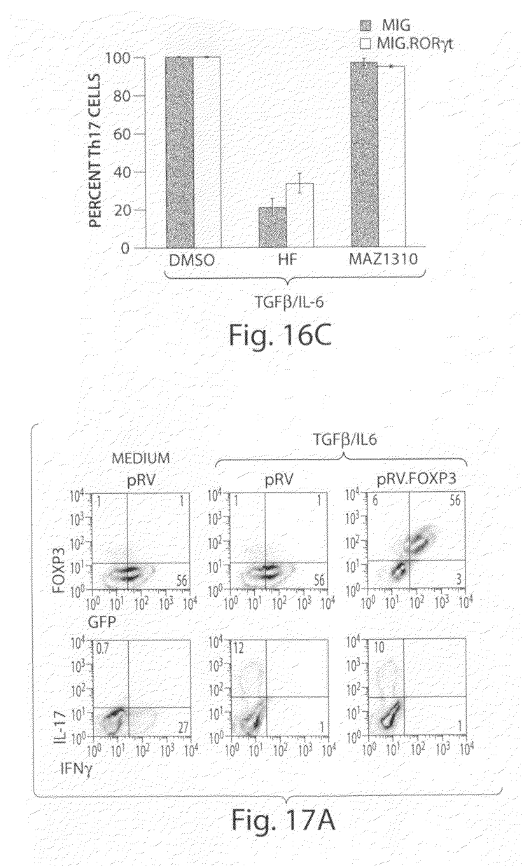

STAT3 promotes Th17 lineage commitment through the induction of the orphan nuclear receptors RORγt and RORγ (Ivanov, I I et al. The orphan nuclear receptor RORgammat directs the differentiation program of proinflammatory IL-17+T helper cells. Cell 126, 1121-33 (2006); Yang, X. O. et al. STAT3 regulates cytokine-mediated generation of inflammatory helper T cells. J Biol Chem 282, 9358-63 (2007); and Yang, X. O. et al. T Helper 17 Lineage Differentiation Is Programmed by Orphan Nuclear Receptors RORalpha and RORgamma. Immunity 28, 29-39 (2008)). Consistent with the finding that HF did not affect STAT3 phosphorylation during the first 12 hours of stimulation, HF did not interfere with the upregulation of RORγt or RORγ during Th17 differentiation (FIG. 16A). Moreover, HF inhibited Th17 differentiation as effectively in T cells retrovirally transduced with RORγt-expressing retroviruses as in those transduced with empty retroviruses (FIGS. 16B, 16C).

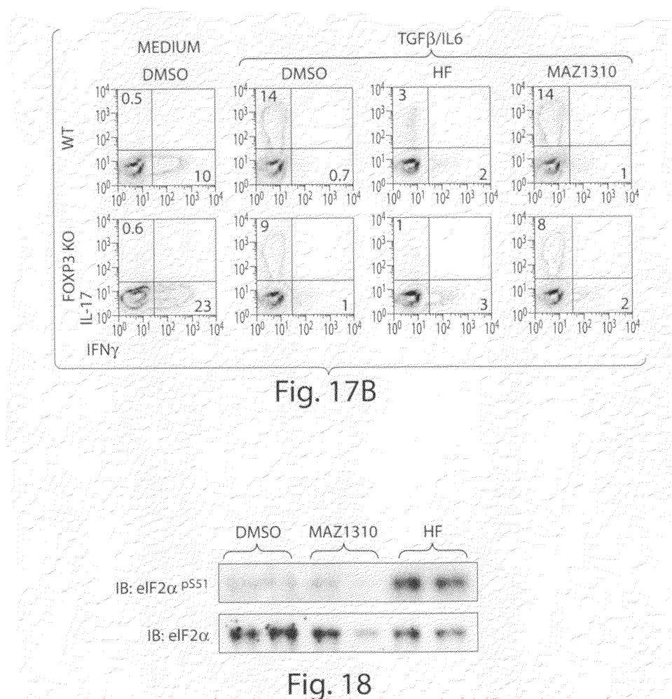

T cells differentiated in the presence of HF showed enhanced Foxp3 expression (FIG. 11E), as expected from the fact that HF inhibits STAT3 signaling and Th17 differentiation. (Yang, X. O. et al. STAT3 regulates cytokine-mediated generation of inflammatory helper T cells. J Biol Chem 282, 9358-63 (2007)). This result demonstrated that HF redirects developing Th17 cells to the iTreg lineage rather than simply blocking their effector function. However, upregulation of Foxp3 by HF was neither necessary nor sufficient to inhibit Th17 differentiation: retroviral expression of FOXP3 in T cells did not decrease IL-17 expression induced by TGFβ plus IL-6 (FIG. 17A), though it markedly reduced IL-2 and IFNγ production in T cells cultured under non-polarizing conditions. Moreover, HF strongly repressed IL-17 expression in T cells lacking Foxp3³⁶ (FIG. 17B). Therefore, the inhibitory effects of HF on Th17 differentiation are not exerted indirectly through the upregulation of Foxp3. Rather, HF impairs the maintenance of STAT3 phosphorylation in developing Th17 cells, resulting in a reciprocal increase in iTreg cell development.

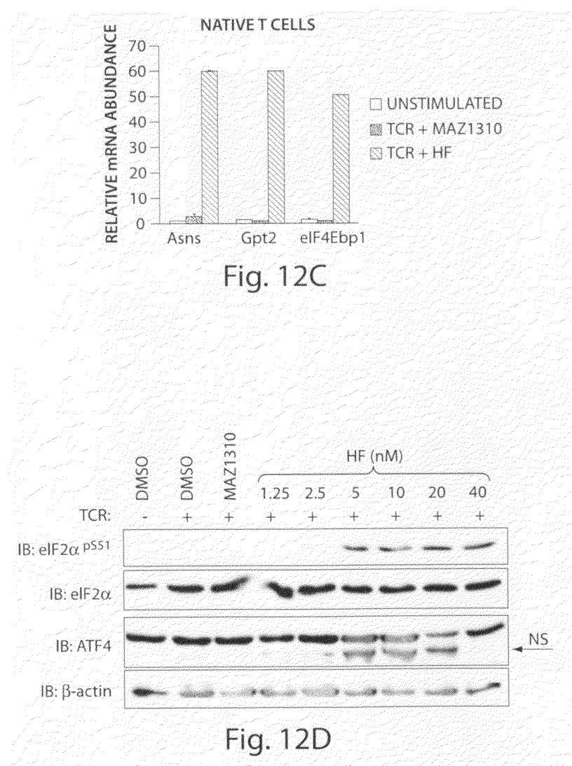

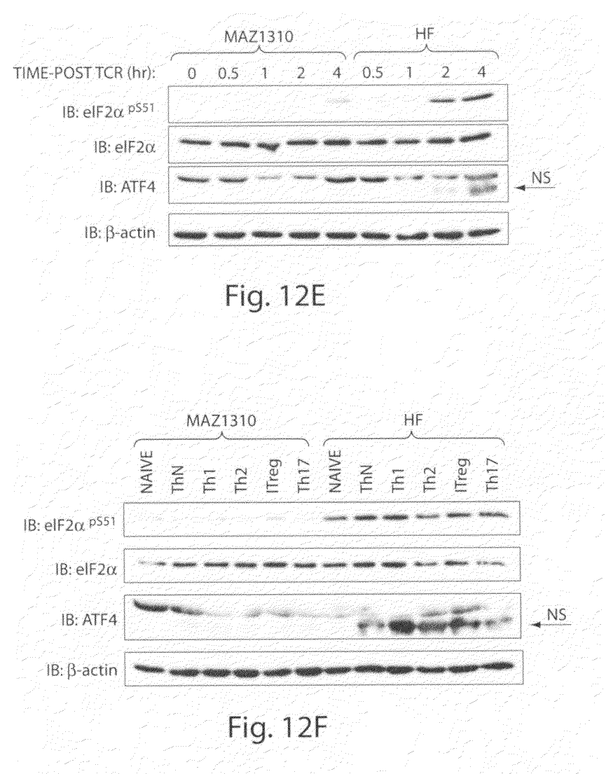

The 12-hour lag period between the addition of HF to T cell cultures and the ensuing effect on STAT3 phosphorylation indicated an indirect effect. To identify more proximal cellular effects of HF treatment, DNA microarrays were used to define the transcriptional profiles of HF- and MAZ1310-treated T cells activated in Th17-priming conditions for 3 or 6 hours. 81 annotated genes that were differentially expressed at both time points in HF- versus MAZ1310-treated cells were identified, the majority of which were upregulated following HF treatment (FIG. 12A, Table 1).

Table 1 lists the gene symbols and names of transcripts that were increased at least 2-fold by HF treatment at both 3 and 6 hours. Mean fold increases±SD from triplicate samples of HF- versus MAZ1310-treated T cells are shown at 3 and 6 hours.

TABLE 1

| Gene symbol | Gene title | HF vs. MAZ1310-3 hr | HF vs. MAZ1310-6 hr |
|---|---|---|---|
| Gpt2 | glutamic pyruvate transaminase (alanine aminotransferase) | 10.0 ± 1.2 | 16.7 ± 2.2 |
| Trib3 | Tribbles homolog 3 (Drosophila) | 7.1 ± 2.0 | 18.5 ± 8.5 |
| Eif4Ebp1 | eukaryotic translation initiation factor 4E binding protein 1 | 6.8 ± 1.8 | 5.3 ± 0.3 |
| Asns | asparagine synthetase | 6.1 ± 1.2 | 7.1 ± 0.5 |
| Ddit3 | DNA-damage inducible transcript 3 | 5.6 ± 1.1 | 5.0 ± 0.7 |
| Pck2 | phosphoenolpyruvate carboxykinase 2 (mitochondrial) | 4.9 ± 0.8 | 7.4 ± 0.9 |
| Pycr1 | pyrroline-5-carboxylate reductase 1 | 4.6 ± 0.7 | 6.6 ± 0.4 |
| Cebpb | CCAAT/enhancer binding protein (C/EBP), beta | 3.9 ± 0.5 | 8.0 ± 0.2 |
| Phgdh | 3-phosphoglycerate dehydrogenase | 3.8 ± 0.9 | 4.2 ± 0.3 |
| Psph | phosphoserine phosphatase | 3.5 ± 0.4 | 3.4 ± 0.3 |
| Xist | inactive X specific transcripts | 3.5 ± 1.7 | 2.1 ± 0.7 |
| Pdcd1lg2 | programmed cell death 1 ligand 2 | 3.2 ± 0.7 | 2.5 ± 0.3 |
| Vegfa | vascular endothelial growth factor A | 3.2 ± 0.2 | 5.8 ± 0.5 |
| Cldn12 | claudin 12 | 3.2 ± 0.7 | 4.6 ± 0.5 |
| Slc1a4 | solute carrier family 1 (glutamate/neutral amino acid transporter), member 4 | 3.2 ± 0.9 | 4.6 ± 0.4 |
| Atf3 | activating transcription factor 3 | 3.0 ± 0.1 | 3.2 ± 0.5 |
| Ncoa7 | nuclear receptor coactivator 7 | 3.0 ± 0.3 | 3.2 ± 0.5 |
| Aars | alanyl-tRNA synthetase | 2.7 ± 0.4 | 2.6 ± 0.2 |
| Sesn2 | sestrin 2 | 2.6 ± 0.3 | 2.3 ± 0.2 |
| Cebpg | CCAAT/enhancer binding protein (C/EBP), gamma | 2.5 ± 0.5 | 3.1 ± 0.5 |
| Slc5a9 | solute carrier family 5 (neurotransmitter transporter, glycine), member 9 | 2.4 ± 0.3 | 5.7 ± 0.6 |
| Herpud1 | homocysteine-inducible, endoplasmic reticulum stress-inducible, ubiquitin-like | 2.4 ± 0.3 | 2.7 ± 0.1 |
| | domain member 1 | | |
| Trim12 | tripartite motif protein 12 | 2.4 ± 0.1 | 4.9 ± 0.7 |
| Clic4 | chloride intracellular channel 4 (mitochondrial) | 2.4 ± 0.2 | 2.8 ± 0.2 |
| Atf5 | activating transcription factor 5 | 2.4 ± 0.1 | 8.9 ± 1.0 |
| Mpa2l | macrophage activation 2 like | 2.3 ± 0.3 | 7.3 ± 1.7 |
| Aff1 | AF4/FMR2 family, member 1 | 2.3 ± 0.4 | 2.6 ± 0.3 |
| Lars | leucyl-tRNA synthetase | 2.3 ± 0.3 | 2.1 ± 0.0 |
| Cth | cystathionase (cystathionine gamma-lyase) | 2.2 ± 0.7 | 16.0 ± 1.6 |
| Chd2 | chromodomain helicase DNA binding protein 2 | 2.2 ± 0.3 | 2.5 ± 0.5 |
| Cars | cysteinyl-tRNA synthetase | 2.2 ± 0.4 | 2.2 ± 0.3 |
| Slamf7 | SLAM family member 7 | 2.2 ± 0.4 | 2.1 ± 0.2 |
| Cxcl10 | chemokine (C-X-C motif) ligand 10 | 2.1 ± 0.3 | 2.1 ± 0.1 |
| Psat1 | phosphoserine aminotransferase 1 | 2.1 ± 0.5 | 2.6 ± 0.0 |
| Aldh18a1 | aldehyde dehydrogenase 18 family, member A1 | 2.1 ± 0.5 | 2.7 ± 0.2 |
| Pycs | 1-@pyrroline-5-carboxylate synthetase | 2.1 ± 0.2 | 2.3 ± 0.1 |
| Cd274 | CD274 antigen | 2.1 ± 0.2 | 2.0 ± 0.1 |
| D8Ertd56e | DNA segment. Chr 8, ERATO Doi 56, expressed | 2.1 ± 0.3 | 3,1 ± 0.8 |
| Irf1 | interferon regulatory factor 1 | 2.0 ± 0.3 | 2.6 ± 0.2 |
| Pvr | poliovirus receptor | 2.0 ± 0.3 | 2.0 ± 0.1 |
| Nfkbiz | Nuclear factor of kappa light polypeptide gene enhancer in B-cells inhibitor, zeta | 2.0 ± 0.3 | 1.9 ± 0.3 |
| Icam1 | intercellular adhesion molecule | 2.0 ± 0.1 | 2.8 ± 0.3 |
| Slc14a1 | solute carrier family 14 (urea transporter), member 1 | 2.0 ± 0.1 | 6.6 ± 0.4 |
| Sars1 | seryl-aminoacyl-tRNA synthetase | 2.0 ± 0.3 | 2.3 ± 0.1 |
| Slc7a3 | solute carrier family 7 (cationic amino acid transporter, y+ system), member 3 | 2.0 ± 0.2 | 6.5 ± 0.9 |

Among the HF-inducible transcripts, a large number of genes functionally associated with amino acid synthesis and transport, as well as protein synthesis, were observed. (FIG. 12A, Table 1). Similar gene expression profiles have been observed during cellular responses to amino acid starvation. (Fafournoux, P., Bruhat, A. & Jousse, C. Amino acid regulation of gene expression. Biochem J 351, 1-12 (2000); and Peng, T., Golub, T. R. & Sabatini, D. M. The immunosuppressant rapamycin mimics a starvation-like signal distinct from amino acid and glucose deprivation. Mol Cell Biol 22, 5575-84 (2002)). Insufficient cellular levels of amino acids lead to the accumulation of uncharged tRNAs that, in turn, activate the amino acid response (AAR) pathway via the protein kinase GCN2. Activated GCN2 phosphorylates and inhibits eukaryotic translation initiation factor 2A (eIF2α), thereby reducing overall protein translation, while specifically enhancing translation of the transcription factor ATF4. (Harding, H. P. et al. An integrated stress response regulates amino acid metabolism and resistance to oxidative stress. Mol Cell 11, 619-33 (2003); and Harding, H. P. et al. Regulated translation initiation controls stress-induced gene expression in mammalian cells. Mol Cell 6, 1099-108 (2000)). A number of stress-induced genes reportedly regulated by ATF4 in mouse embryonic fibroblasts (Harding, H. P. et al. An integrated stress response regulates amino acid metabolism and resistance to oxidative stress. Mol Cell 11, 619-33 (2003)) were over-represented among the genes induced by HF treatment in T cells (FIG. 12B, Table 2).

Table 2 lists the probe IDs of known stress response genes. This table provides the Affymetrix probe IDs and gene names previously identified as ATF4 responsive during tunicamycin-induced ER stress in mouse embryonic fibroblasts (see H. P. Harding, et al. Mol Cell, 2003, 11(3): 619-33).

TABLE 2

| Affymetrix probe ID | Gene Name |
|---|---|
| 1433966_x_at | asparagine synthetase |
| 1451095_at | asparagine synthetase |
| 1451083_s_at | alanyl-tRNA synthetase |
| 1423685_at | alanyl-tRNA synthetase |
| 1435154_at | similar to solute carrier family 7 (cationic amino acid transporter, y+ system), member 3 |
| 1454991_at | solute carrier family 7 (cationic amino acid transporter, y+ system), member 1 |
| 1454992_a | solute carrier family 7 (cationic amino acid transporter, y+ system), member 1 |
| 1421533_at | solute carrier family 7 (cationic amino acid transporter, y+ system), member 1 |
| 1421093_at | solute carrier family 7 (cationic amino acid transporter, y+ system), member 10 |
| 1420413_at | solute carrier family 7 (cationic amino acid transporter, y+ system), member 11 |
| 1443536_at | solute carrier family 7 (cationic amino acid transporter, y+ system), member 11 |
| 1419579_at | solute carrier family 7 (cationic amino acid transporter, y+ system), member 12 |
| 1422648_at | solute carrier family 7 (cationic amino acid transporter, y+ system), member 2 |
| 1426008_a_at | solute carrier family 7 (cationic amino acid transporter, y+ system), member 2 |
| 1440506_at | Solute carrier family 7 (cationic amino acid transporter, y+ system), member 2 |
| 1417022_at | solute carrier family 7 (cationic amino acid transporter, y+ system), member 3 |
| 1426069_s_at | solute carrier family 7 (cationic amino acid transporter, y+ system), member 4 |
| 1426068_at | solute carrier family 7 (cationic amino acid transporter, y+ system), member 4 |
| 1436776_x_at | solute carrier family 7 (cationic amino acid transporter, y+ system), member 4 |
| 1418326_at | solute carrier family 7 (cationic amino acid transporter, y+ system), member 5 |
| 1460541_at | solute carrier family 7 (cationic amino acid transporter, y+ system), member 6 |
| 1433467_at | solute carrier family 7 (cationic amino acid transporter, y+ system), member 6 |
| 1417392_a_at | solute carrier family 7 (cationic amino acid transporter, y+ system), member 7 |
| 1447181_s_at | solute carrier family 7 (cationic amino acid transporter, y+ system), member 7 |
| 1417929_at | solute carrier family 7 (cationic amino acid transporter, y+ system), member 8 |
| 1448783_at | solute carrier family 7 (cationic amino acid transporter, y+ system), member 9 |
| 1431740_at | solute carrier family 7, (cationic amino acid transporter, y+ system) member 13 |
| 1449301_at | solute carrier family 7, (cationic amino acid transporter, y+ system) member 13 |
| 1456003_a_at | solute carrier family 1 (glutamate/neutral amino acid transporter), member 4 |
| 1423550_at | solute carrier family 1 (glutamate/neutral amino acid transporter), member 4 |
| 1423549_at | solute carrier family 1 (glutamate/neutral amino acid transporter), member 4 |
| 1440379_at | solute carrier family 1 (neutral amino acid transporter), member 5 |
| 1416629_at | solute carrier family 1 (neutral amino acid transporter), member 5 |
| 1422757_at | solute carrier family 5 (neutral amino acid transporters, system A), member 4b |
| 1419253_at | methylenetatrahydrofolate dehydrogenase (NAD+ dependent), methenyltetrahydrofolate cyclohydrolase |
| 1419254_at | methylenetatrahydrofolate dehydrogenase (NAD+ dependent), methenyltetrahydrofolate cyclohydrolase |
| 1456653_a_at | methylenetatrahydrofolate dehydrogenase (NADP+ dependent) 1-like |
| 1415917_at | methylenetatrahydrofolate dehydrogenase (NADP+ dependent), methenyltetrahydrofolate cyclohydrolase, |
| | formyltetrahydrofolate synthase |
| 1415916_a_at | methylenetatrahydrofolate dehydrogenase (NADP+ dependant), methenyltetrahydrofolate cyclohydrolase, |
| | formyltetrahydrofolate synthase |
| 1436704_x_at | methylenetatrahydrofolate dehydrogenase (NADP+ dependent), methenyltetrahydrofolate cyclohydrolase, |
| | formyltetrahydrofolate synthase |
| 1451064_a_at | phosphoserine aminotransferase 1 |
| 1454607_s_at | phosphoserine aminotransferase 1 |
| 1415673_at | phosphoserine phosphatase |
| 1417562_at | eukaryotic translation initiation factor 4E binding protein 1 |
| 1417563_at | eukaryotic translation initiation factor 4E binding protein 1 |
| 1434976_x_at | eukaryotic translation initiation factor 4E binding protein 1 |
| 1428666_at | asparaginyl-tRNA synthetase |
| 1452866_at | asparaginyl-tRNA synthetase |
| 1415694_at | tryptophanyl-tRNA synthetase |
| 1437832_x_at | tryptophanyl-tRNA synthetase |
| 1434813_x_at | tryptophanyl-tRNA synthetase |
| 1425106_a_at | tryptophanyl-tRNA synthetase |
| 1430111_a_at | branched chain aminotransferase 1, cytosolic |
| 1450871_a_at | branched chain aminotransferase 1, cytosolic |
| 1425764_a_at | branched chain aminotransferase 2, mitochondrial |
| 1460323_at | threonyl-tRNA synthetase |
| 1436856_x_at | threonyl-tRNA synthetase-like 1 |
| 1431125_a_at | threonyl-tRNA synthetase-like 1 |
| 1434738_at | threonyl-tRNA synthetase-like 2 |
| 1448403_at | leucyl-tRNA synthetase |
| 1418892_at | ras homolog gene family, member J |
| 1448594_at | WNT1 inducible signaling pathway protein 1 |
| 1448593_at | WNT1 inducible signaling pathway protein 1 |
| 1425458_a_at | growth factor receptor bound protein 10 |
| 1425457_a_at | growth factor receptor bound protein 10 |
| 1430164_a_at | growth factor receptor bound protein 10 |
| 1440935_at | Growth factor receptor bound protein 10, mRNA (cDNA clone MGC:28740 IMAGE:4481345) |
| 1428365_a_at | protease, serine, 15 |
| 1416168_at | serine (or cysteine) peptidase inhibitor, clade F, member 1 |
| 1453724_a_at | serine (or cysteine) peptidase inhibitor, clade F, member 1 |
| 1450196_s_at | glycogen synthase 1, muscle///glycogen synthase 3, brain |
| 1438606_a_at | chloride intracellular channel 4 (mitochondrial) |
| 1423393_at | chloride intracellular channel 4 (mitochondrial) |
| 1423392_at | chloride intracellular channel 4 (mitochondrial) |
| 1422018_at | human immunodeficiency virus type 1 enhancer binding protein 2 |
| 1434904_at | Human immunodeficiency virus type 1 enhancer binding protein 2 (Hivep2), mRNA |
| 1444990_at | Human immunodeficiency virus type 1 enhancer binding protein 2 (Hivep2), mRNA |

These analyses indicated that at least a portion of the transcriptional response to HF is mediated by ATF4. Furthermore, quantitative real-time PCR (qPCR) confirmed that at least three known AAR-associated genes (Asns, Gpt2, eIF4Ebp1) were induced by HF treatment within 4 hours of T cell activation (FIG. 12C).

To address directly whether HF activates the AAR pathway, eIF2α phosphorylation and ATF4 protein levels in HF-treated T cells were examined. HF induced detectable eIF2α phosphorylation at 2.5 nM, and this effect plateaued at 5-10 nM HF (FIG. 12D). ATF4 expression levels were highest in T cells treated with 5-10 nM HF and were reduced in cells treated with higher concentrations of HF (20-40 nM) (FIG. 12D), demonstrating a positive correlation between the concentrations of HF that induce ATF4 expression and those that selectively inhibit Th17 differentiation (FIG. 10A). In kinetic analyses, eIF2α phosphorylation in HF-treated cells reached maximum levels by 2 hours and ATF4 protein continued to accumulate until 4 hours (FIG. 12E), indicating that HF activates the AAR pathway before any detectable effects on STAT3 phosphorylation or IL-17 production are observed. AAR activation was a general consequence of HF treatment: HF induced eIF2α phosphorylation not only in T cells activated in Th17-priming conditions, but also in resting naïve T cells and T cells activated in ThN, Th1, Th2 and iTreg polarizing conditions (FIG. 12F). HF treatment also increased eIF2α phosphorylation in cultured fibroblasts (FIG. 18), and microarray analyses of HF-treated fibroblasts revealed a pattern of early gene induction similar to that seen in T-cells, demonstrating that activation of the AAR pathway by HF is not a T cell-specific effect. HF treatment induced ATF4 expression in all differentiated T cells but not in naïve T cells (FIG. 12F); this result reflects the low metabolic rate and relatively inefficient translation capacity of naïve T cells. (Rathmell, J. C., Elstrom, R. L., Cinalli, R. M. & Thompson, C. B. Activated Akt promotes increased resting T cell size, CD28-independent T cell growth, and development of autoimmunity and lymphoma. Eur J Immunol 33, 2223-32 (2003)). Thus, the rapid activation of the AAR pathway by HF underlies both its selective inhibition of Th17 differentiation and its effects on fibroblasts. (Pines, M. & Nagler, A. Halofuginone: a novel antifibrotic therapy. Gen Pharmacol 30, 445-50 (1998)).

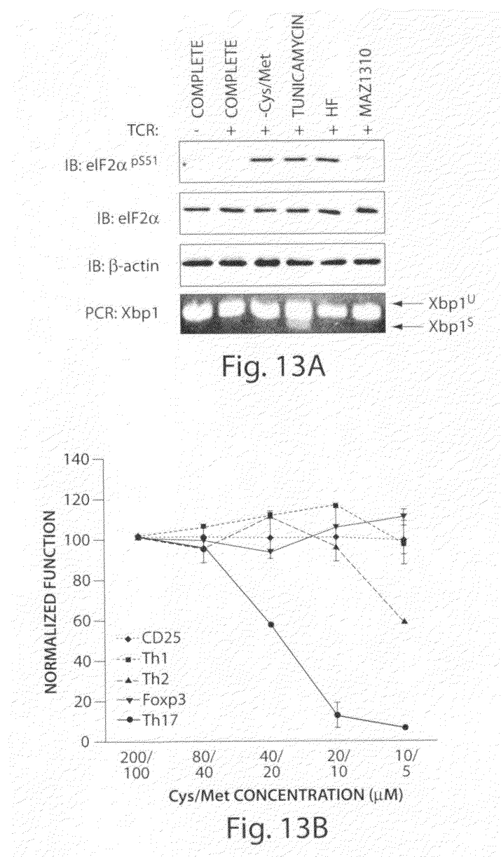

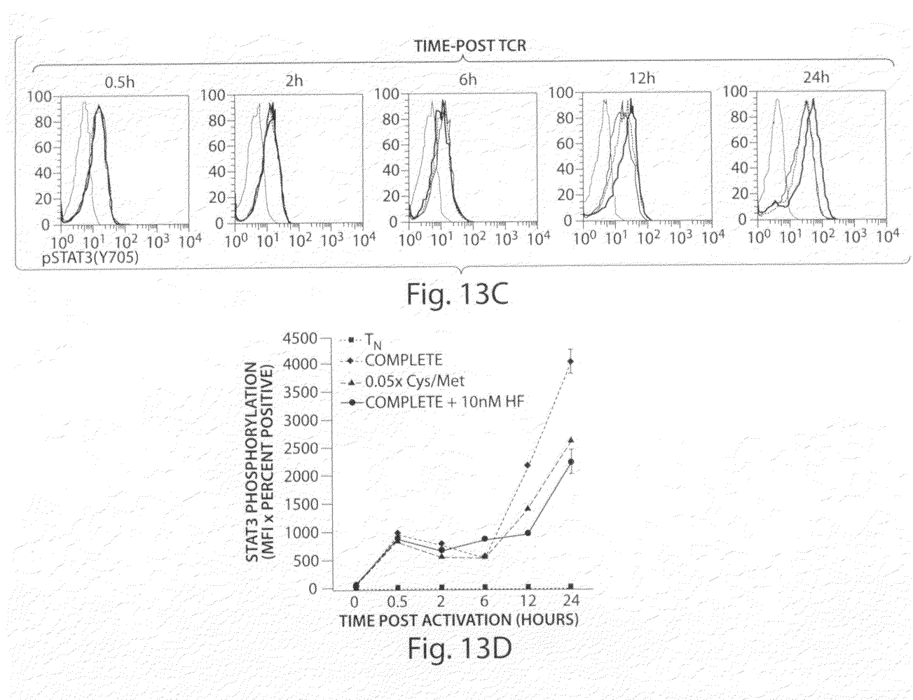

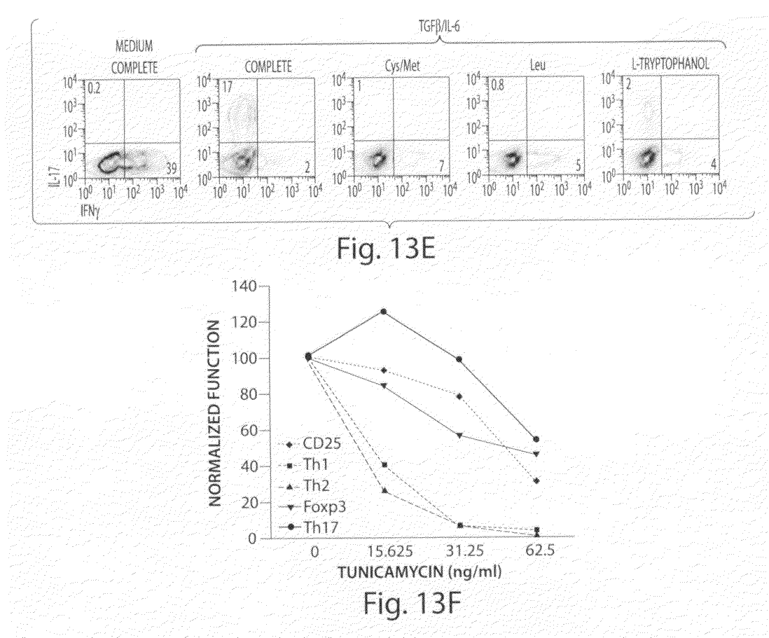

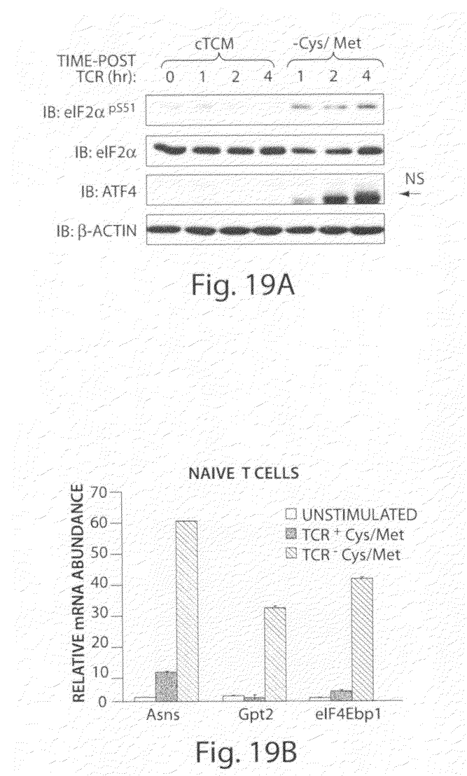

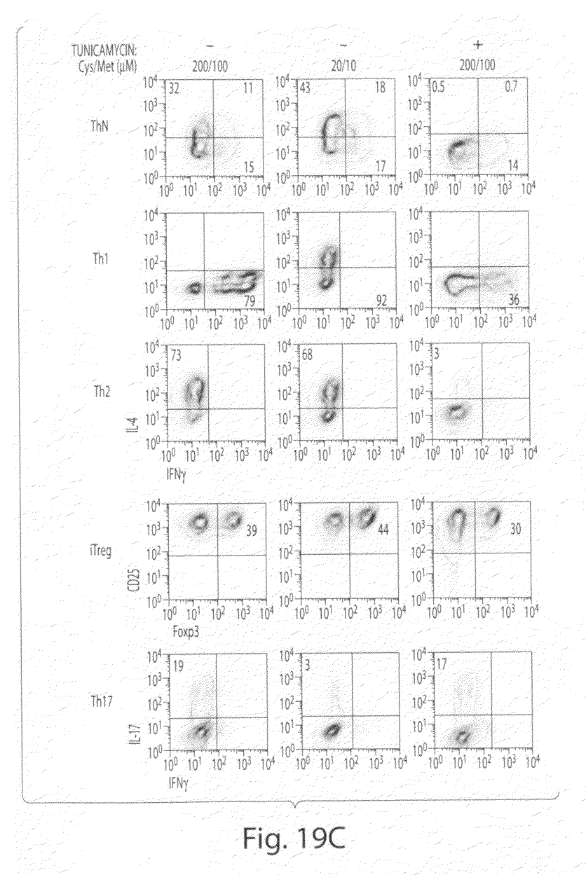

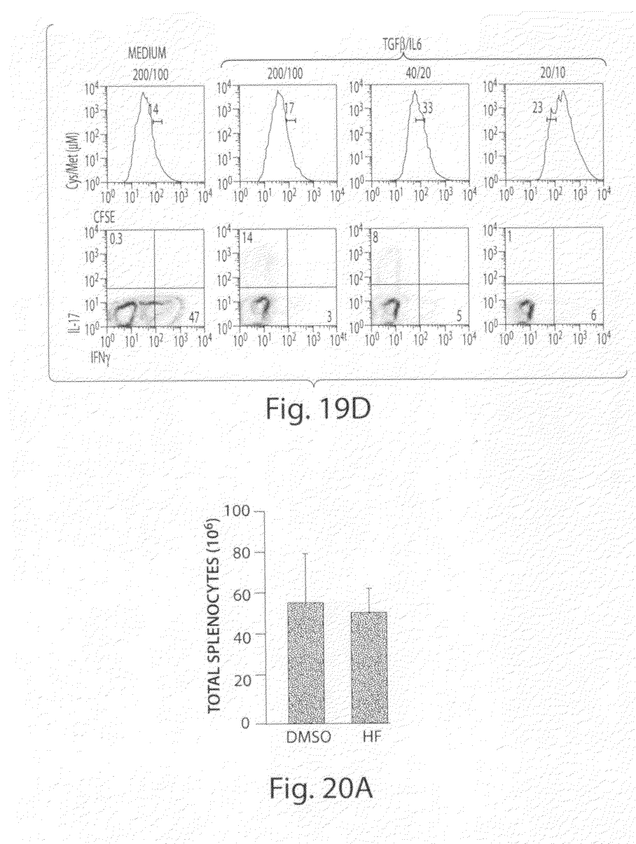

A variety of other cellular stresses (ER stress, oxidative stress, viral infection) also result in eIF2α phosphorylation and ATF4 translation, a phenomenon termed the integrated stress response (ISR). (Harding, H. P. et al. An integrated stress response regulates amino acid metabolism and resistance to oxidative stress. Mol Cell 11, 619-33 (2003); and Harding, H. P. et al. Regulated translation initiation controls stress-induced gene expression in mammalian cells. Mol Cell 6, 1099-108 (2000)). Individual stressors, however, can also activate stress type-specific pathways. For instance, the unfolded protein response (UPR), which is activated by ER stress, results in expression of the transcription factor Xbp-1 through a mechanism involving IRE-1-dependent splicing, as well as nuclear translocation of the ER-sequestered transcription factor ATF6 in addition to eIF2α phosphorylation catalyzed by the protein kinase Perk. (Ron, D. & Walter, P. Signal integration in the endoplasmic reticulum unfolded protein response. Nat Rev Mol Cell Biol 8, 519-29 (2007); Brunsing, R. et al. B- and T-cell development both involve activity of the unfolded protein response pathway. J Biol Chem 283, 17954-61 (2008); and Lin, J. H. et al. IRE1 signaling affects cell fate during the unfolded protein response. Science 318, 944-9 (2007)). Xbp-1 and ATF6, in turn, upregulate ER chaperones such as GRP78/BiP and calreticulin, whose expression is specific to the UPR and independent of the eIF2α/ATF4 ISR pathway (Ron, D. & Walter, P. Signal integration in the endoplasmic reticulum unfolded protein response. Nat Rev Mol Cell Biol 8, 519-29 (2007); and Lee, A. H., Iwakoshi, N. N. & Glimcher, L. H. XBP-1 regulates a subset of endoplasmic reticulum resident chaperone genes in the unfolded protein response. Mol Cell Biol 23, 7448-59 (2003)). However, HF did not induce the expression of these and other hallmark ER stress response genes. To delineate the stress response pathway activated by HF, the effects of amino acid deprivation, tunicamycin (an inducer of ER stress), and HF treatment were examined in activated T cells. As expected, cells deprived of cysteine (Cys) and methionine (Met) displayed eIF2α phosphorylation, ATF4 expression and upregulation of AAR-associated genes but did not induce Xbp-1 splicing (FIGS. 13A, 19A, 19B). In contrast, tunicamycin treatment induced eIF2α phosphorylation and ATF4 expression together with Xbp-1 splicing (FIG. 13A), as characteristic of the UPR. The effects of HF treatment resembled those of amino acid starvation, inducing eIF2α phosphorylation without promoting Xbp-1 splicing (FIG. 13A). Taken together, these data indicate that HF specifically induces an AAR.

These results led to studies designed to investigate the effects of amino acid starvation on Th17 differentiation and STAT3 activation. It was found that the functional consequences of Cys/Met-deprivation were remarkably similar to those of HF treatment in T cells. Cys/Met deprivation profoundly and selectively impaired Th17 differentiation in a manner directly related to the concentration of these amino acids in the culture medium: T cells cultured under limiting Cys/Met concentrations showed greatly diminished Th17 differentiation but upregulated CD25 expression and differentiated into Th1, Th2 and iTreg subsets as effectively as T cells cultured in complete medium (FIGS. 13B and 19C). As shown for HF (FIG. 10E), inhibition of IL-17 expression by amino acid starvation was unrelated to the number of cell divisions, cell survival or proliferation (FIG. 19D). As observed for HF, Cys/Met-deprivation did not affect the early phase of STAT3 phosphorylation but impaired the maintenance of STAT3 phosphorylation (FIGS. 13C, 13D). Moreover L-tryptophanol, a tryptophan derivative that competitively inhibits tryptophanyl-tRNA loading, or limiting concentrations of a different amino acid, leucine, also impaired IL-17 production (FIG. 13E), indicating that inhibition of Th17 differentiation is a general consequence of amino acid starvation. The mammalian target of rapamycin (mTOR) pathway represents a second, complementary mechanism through which cells respond to amino acid availability (Fingar, D. C. & Blenis, J. Target of rapamycin (TOR): an integrator of nutrient and growth factor signals and coordinator of cell growth and cell cycle progression. Oncogene 23, 3151-71 (2004)) but the early transcriptional responses induced by HF and the mTOR inhibitor rapamycin are distinct (Table 1) (Peng, T., Golub, T. R. & Sabatini, D. M. The immunosuppressant rapamycin mimics a starvation-like signal distinct from amino acid and glucose deprivation. Mol Cell Biol 22, 5575-84 (2002)), and HF did not inhibit signaling downstream of mTOR in fibroblasts.

To test whether inhibition of IL-17 expression was specific to stress induced by amino acid starvation, studies were designed to evaluate whether tunicamycin would influence T cell activation and differentiation. Surprisingly, tunicamycin treatment had little influence on IL-17 expression in T cells (FIGS. 13F, 19C), but instead preferentially impaired Th1 and Th2 differentiation (FIGS. 13F, 19C). These data indicate that individual stress response pathways regulate distinct aspects of T cell differentiation and effector function, but also indicate that eIF2α phosphorylation and ATF4 translation (shared consequences of both AAR and UPR) are not sufficient to explain the selective regulation of Th17 differentiation by HF or amino acid deprivation.

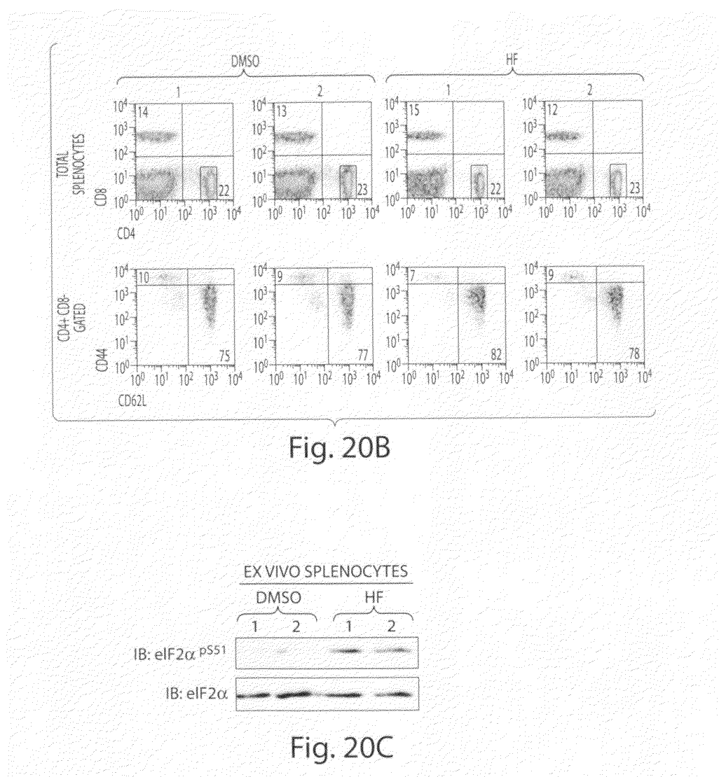

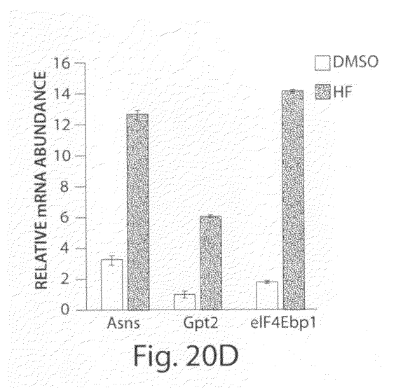

Studies were conducted to evaluate the effect of HF treatment in mice. HF rapidly activates eIF2α phosphorylation and AAR-associated gene expression in splenocytes isolated from mice treated with HF (FIG. 20).

It is shown here that Th17 differentiation is particularly susceptible to stress induced by amino acid deprivation, whereas ER stress blunts Th1 and Th2 differentiation. By inducing the AAR response, HF—an established anti-fibrotic drug—imparts a selective block of Th17 differentiation in both human and mouse T cells. The results presented herein demonstrate HF is useful for therapeutic and/or cosmetic intervention in pathologies and disorders such as cellulite in a subject in need thereof. For example, HF is useful for therapeutic and/or cosmetic intervention of the progression and/or formation of cellulite. In addition, the results presented herein demonstrate that the pathways involved in cellular stress responses are useful targets for the rational design of therapeutics.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

All publications and patent documents cited herein are incorporated herein by reference as if each such publication or document was specifically and individually indicated to be incorporated herein by reference.

What is claimed is:

1. A method for preventing or decreasing the appearance of cellulite in a subject exhibiting cellulite in at least one tissue site, said method comprising administering a selective Th17 inhibitor.

2. The method of claim 1, wherein said inhibitor modulates the development of Th17 T cells in a subject.

3. The method of claim 1, wherein said selective Th17 inhibitor comprises a compound of formula I: 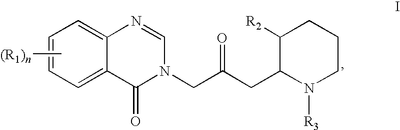
or a salt, isomer, derivative, analog, solvate enantiomer, diasteriomer or multimer thereof,
wherein: R₁ is selected from hydrogen, halogen, nitro, benzo, lower alkyl, phenyl and lower alkoxy;
R₂ is selected from hydroxy, acetoxy, and lower alkoxy,
R₃ is selected from hydrogen lower alkoxy-carbonyl and lower alkenoxy-carbonyl, and
n is selected from 1, 2, 3 and 4;
in an amount effective to modulate the development of Th17 cells from precursor helper T cells in a subject.

4. The method of claim 1, wherein said compound is febrifugine, halofuginone or a derivative thereof.

5. The method of claim 1, wherein said compound inhibits maturation of myofibroblasts.

6. The method of claim 1, wherein said compound inhibits one or more biological activities of myofibroblasts.

7. The method of claim 6, wherein said biological activity is selected from expression of actin-containing stress fibers, expression and organization of fibronectin into fibrils, and formation of large fibronexus adhesion complexes.

8. The method of claim 1, wherein said compound is formulated as a film, membrane, foam, gel, cream or injectable composition.

9. The method of claim 1, further comprising the step of:
(c) exposing said tissue site to a laser.

10. The method of claim 1, further comprising the step of:
(c) exposing said tissue site to mechanical manipulation.

11. The method of claim 1, wherein said selective Th17 inhibitor is administered in conjunction with a second agent.

12. The method of claim 11, wherein said second agent is a statin or retinoic acid.

13. The method of claim 11, wherein said inhibitor and said second agent synergistically prevent or decrease the appearance of cellulite.

14. A method for inducing an amino acid starvation response (AAR) in a subject in need thereof, said method comprising administering to said patient a compound selected from a compound that selectively inhibits the development of Th17 T cells or a compound that induces intracellular accumulation of uncharged transfer ribonucleic acids (tRNAs), wherein the compound is administered in an amount effective to induce AAR in said subject.

15. The method of claim 14, wherein said compound is a compound of formula I: 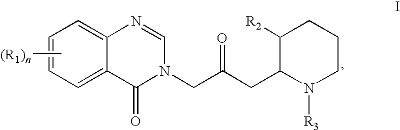
or a salt, isomer, derivative, analog, solvate, enantiomer, diasteriomer or multimer thereof,
wherein: R₁ is selected from hydrogen, halogen, nitro, benzo, lower alkyl, phenyl and lower alkoxy;
R₂ is selected from hydroxy, acetoxy, and lower alkoxy,
R₃ is selected from hydrogen lower alkoxy-carbonyl and lower alkenoxy-carbonyl, and
n is selected from 1, 2, 3 and 4;
in an amount effective to effective to AAR in a subject.

16. The method of claim 14, wherein said compound is febrifugine, halofuginone, histidinol, tryptophanol, reveromycin, or a derivative thereof.

17. The method of claim 14, wherein said compound is formulated for systemic administration.

18. The method of claim 14, wherein said compound is formulated as an injectable composition.


SEQUENCE LISTING (8 sequences)

SEQ ID NO: 1 - DNA, 27 bp, Artificial Sequence
  chemically synthesized
tgactgcctttccgtgcagtgtctgag

SEQ ID NO: 2 - DNA, 28 bp, Artificial Sequence
  chemically synthesized
acagccaagcggtgaaagccaaagcagc

SEQ ID NO: 3 - DNA, 27 bp, Artificial Sequence
  chemically synthesized
tagtcacagcagcgctgcagccgaagc

SEQ ID NO: 4 - DNA, 27 bp, Artificial Sequence
  chemically synthesized
tactccaccgccttcacctgcgggttc

SEQ ID NO: 5 - DNA, 28 bp, Artificial Sequence
  chemically synthesized
accaggattatctatgaccggaaatttc

SEQ ID NO: 6 - DNA, 27 bp, Artificial Sequence
  chemically synthesized
tgggaggctcatcgctggtagggctag

SEQ ID NO: 7 - DNA, 25 bp, Artificial Sequence
  chemically synthesized
gggggctataagttctttgctgacc

SEQ ID NO: 8 - DNA, 27 bp, Artificial Sequence
  chemically synthesized
tccaacacttcgagaggtccttttcac